(19) United States
(12) Patent Application Publication
(10) Pub. No.: US 2017/0095237 A1
(43) Pub. Date: Apr. 6, 2017

(54) SYSTEM AND METHOD FOR EXTRACTION OF HAIR FOLLICLE

(71) Applicant: PiloFocus, Inc., New York, NY (US)

(72) Inventors: Carlos K. Wesley, New York, NY (US); Trevor K. Lewis, Lehi, UT (US)

(21) Appl. No.: 15/380,604

(22) Filed: Dec. 15, 2016

Related U.S. Application Data
(63) Continuation of application No. 14/273,105, filed on May 8, 2014.
Application No. 14/273,105 is a continuation-in-part of application No. 13/654,252, filed on Oct. 17, 2012, now Pat. No. 8,998,931.
Application No. 14/273,105 is a continuation-in-part of application No. 13/496,905, filed on Apr. 30, 2012, now Pat. No. 9,364,252.
(60) Provisional application No. 61/821,098, filed on May 8, 2013.
(60) Provisional application No. 61/673,143, filed on Jul. 18, 2012.
(60) Provisional application No. 61/547,898, filed on Oct. 17, 2011.
(60) Provisional application No. 61/243,271, filed on Sep. 17, 2009.

(51) Int. Cl. A61B 17/00; A61B 90/13; A61B 17/3205; A61B 90/20; A61B 17/3209

(52) U.S. Cl. CPC A61B 17/00; A61B 90/20; A61B 17/32093; A61B 17/32053; A61B 90/13; A61B 2017/00752; A61B 2090/373

(57) ABSTRACT

Systems and methods for extracting hair follicle from underneath an external surface of the skin are provided. The systems may include a first member and a counter pressure device. The first member may define a first bore and may have a longitudinal axis. A portion of the first member may be configured to be moved below an external surface of a skin. The first member may be configured to translate at least in a first direction along the longitudinal axis through a tissue and rotate about the longitudinal axis. The counter pressure device may be configured to interface with at least the external surface of the skin such that the tissue is disposed between the first member and the counter pressure device. The translation and rotation of the first member may result in cutting at least a portion of the tissue.

### CROSS-REFERENCE TO RELATED APPLICATIONS

This application is a Continuation and claims the benefit of U.S. patent application Ser. No. 14/273,105 filed May 8, 2014, entitled SYSTEM AND METHOD FOR EXTRACTION OF HAIR FOLLICLE, which in turn claims benefit of expired U.S. Provisional Patent Application No. 61/821,098 filed May 8, 2013 entitled HAIR RESTORATION.

U.S. patent application Ser. No. 14/273,105 also is a Continuation-in-Part of issued U.S. patent application Ser. No. 13/654,252 filed Oct. 17, 2012, which granted as U.S. Pat. No. 8,998,931 on Apr. 7, 2015, entitled HAIR RESTORATION, which in turn claims priority to and the benefit of expired U.S. Provisional Patent Application No. 61/673,143 filed Jul. 18, 2012, entitled HAIR RESTORATION and priority to and the benefit of expired U.S. Provisional Patent Application No. 61/547,898 filed Oct. 17, 2011, entitled ENDOSCOPIC HAIR RESTORATION.

U.S. patent application Ser. No. 14/273,105 also is a Continuation-in-Part of issued U.S. patent application Ser. No. 13/496,905, filed Apr. 30, 2012, which granted as U.S. Pat. No. 9,364,252 on Jun. 14, 2016, entitled HAIR RESTORATION SURGERY, which in turn is a 371 National Phase Application claiming priority to and the benefit of PCT/US2010/049283 filed Sep. 17, 2010, entitled HAIR RESTORATION SURGERY, which in turn claims priority to and the benefit of expired U.S. Provisional Application No. 61/243,271, filed Sep. 17, 2009.

The above-identified documents are incorporated herein by reference.

### BACKGROUND

Technical Field

The present disclosure relates to technique for extracting a target hair follicle from a donor site. More particularly, but not exclusively, the present disclosure relates to extracting the hair follicle from underneath an external surface of the skin.

Discussion of Related Art

Hair restoration may be carried out by extracting hair follicular units from the patient's donor area and transplanting them in the patient's recipient area, which may be a thinning area. Generally, the donor area is at the back and sides of the scalp.

Conventionally, a strip of tissues may be removed from the donor area. The strip may be then dissected into individual follicular units. The units may then be transplanted into the recipient area. It has been observed that application of this technique results in a linear scar formed at the donor area.

In another conventional technique, a hair follicle is extracted by punching a hole around the hair follicle, and then extracting the hair follicle. The hole is punched through the external surface of the skin. It has been observed that application of this technique may result in dotted scars formed at the donor area. Further, in this technique, the hair follicle may be transected, as the punch may not be aligned with the alignment of the hair follicle.

In light of the foregoing discussion, there may be a need to reduce scarring in the donor site, and reduce transection or denuding of hair follicle during extraction.

After the preparation of follicular unit grafts doctors make tiny holes in the patient's scalp at the recipient area where grafts are placed. The positioning and arrangement of follicular units depends upon aesthetic qualities of a hair transplant, case-by-case basis, depending on the patients' history of hair loss and likelihood of future hair loss.

Francisco Jimenez, MD, Ander Izeta, PhD, and Enrique Poblet, MD. “Morphometric Analysis of the Human Scalp Hair Follicle: Practical Implications for the Hair Transplant Surgeon and Hair Regeneration Studies” Dermatol Surg 4021; 37:58-64. This document is incorporated herein by reference in its entirety.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a perspective view of a system for extracting hair follicles and another system adapter with the first system to enable extraction of hair follicles;

FIG. 1B is another perspective view of the systems of FIG. 1A;

FIG. 1C is a perspective view of a human head;

FIG. 1D is another perspective view of the human head of FIG. 1C in which an incision is made into the skin;

FIG. 1E is yet another perspective view of the system of FIG. 1A;

FIG. 2A is a perspective view of a first member of the system for extracting hair follicles of FIG. 1A;

FIG. 2B is a top view of the first member of FIG. 2A;

FIG. 2C is a back view of the first member of FIG. 2A;

FIG. 2D is a sectional view about axis A-A of the first member of FIG. 2C;

FIG. 2E is another sectional view about axis B-B of the first member of FIG. 2C;

FIG. 2F is a close up top view of a distal portion of the first member of FIG. 2A;

FIG. 2G is a back view of the distal portion of FIG. 2F;

FIG. 2H is a sectional view about axis C-C of the distal portion of FIG. 2G;

FIG. 2I is another sectional view about axis D-D of the distal portion of FIG. 2G;

FIG. 3A is a perspective view of a second member of the system for extracting hair follicles of FIG. 1A;

FIG. 3B is a top view of the second member of FIG. 3A;

FIG. 3C is a sectional view about axis A-A of the second member of FIG. 3B;

FIG. 3D is a close up perspective view of a distal portion of the second member of FIG. 3A;

FIG. 3E is a top view of the distal portion of FIG. 3D;

FIG. 3F is a sectional view about axis B-B of the distal portion of FIG. 3E;

FIG. 4A is a perspective view of an adapter of the system for extracting hair follicles of FIG. 1A;

FIG. 4B is a top view of the adapter of FIG. 4A;

FIG. 4C is a side view of the adapter of FIG. 4A;

FIG. 4D is a sectional view about axis A-A of the adapter of FIG. 4C;

FIG. 5 is a perspective view of a power shaft of the system for extracting hair follicles of FIG. 1A;

FIG. 6A is a perspective view of a carriage of the system for extracting hair follicles of FIG. 1A;

FIG. 6B is a top view of the carriage of FIG. 6A;

FIG. 6C is a sectional view about axis A-A of the carriage of FIG. 6B;

FIG. 7A is a perspective view of a first connecting member of the system for extracting hair follicles of FIG. 1A;

FIG. 7B is a top view of the first connecting member of FIG. 7A;

FIG. 7C is a sectional view about axis A-A of the first connecting member of FIG. 7B;

FIG. 8A is a perspective view of a stop of the system for extracting hair follicles of FIG. 1A;

FIG. 8B is a top view of the stop of FIG. 8A;

FIG. 8C is a sectional view about axis A-A of the stop of FIG. 8B;

FIG. 9 is a perspective view of a first arm of the system for extracting hair follicles of FIG. 1A;

FIG. 10 is a perspective view of a second connecting member of the system for extracting hair follicles of FIG. 1A;

FIG. 11A is a perspective view of a second arm of the system for extracting hair follicles of FIG. 1A;

FIG. 11B is a top view of the second arm of FIG. 11A;

FIG. 11C is a sectional view about axis A-A of the second arm of FIG. 11B;

FIG. 12 is a perspective view of an adjustment sleeve of the system for extracting hair follicles of FIG. 1A;

FIG. 13A is a perspective view of a stationary member of the system for extracting hair follicles of FIG. 1A;

FIG. 13B is a top view of the stationary member of FIG. 13A;

FIG. 13C is a sectional view about axis A-A of the stationary member of FIG. 13B;

FIG. 14 is a perspective view of a second arm cap of the system for extracting hair follicles of FIG. 1A;

FIG. 15A is a perspective view of four paddle link members of the system for extracting hair follicles of FIG. 1A;

FIG. 15B is a perspective view of one of the four paddle link members of FIG. 15A;

FIG. 16A is a perspective view of a pair of first paddle members of the system for extracting hair follicles of FIG. 1A;

FIG. 16B is a perspective view of one of the pair of first paddle members of FIG. 16A;

FIG. 16C is another perspective view of one of the pair of first paddle members of FIG. 16A;

FIG. 17 is a perspective view of a link member pin of the system for extracting hair follicles of FIG. 1A;

FIGS. 18A and 18B are a perspective view of a external supporting member of the system for extracting hair follicles of FIG. 1A;

FIG. 18C is a front view of the external supporting member of FIG. 18A;

FIG. 18D is a sectional view about axis A-A of the external supporting member of FIG. 18C;

FIG. 19A is a front view of a counter pressure device of the system for extracting hair follicles of FIG. 1A;

FIG. 19B is a perspective view of the counter pressure device of FIG. 19A;

FIG. 20 is a perspective view of a guide plate of the system for extracting hair follicles of FIG. 1A;

FIG. 21A is a perspective view of a cover member of the system for extracting hair follicles of FIG. 1A;

FIG. 21B is a top view of the cover member of FIG. 21A;

FIG. 21C is a sectional view about axis A-A of the cover member of FIG. 21B;

FIG. 22A is a perspective view of a tissue stabilizing member of the system for extracting hair follicles of FIG. 1A;

FIG. 22B is a side view of the tissue stabilizing member of FIG. 22A;

FIG. 22C is a sectional view about axis A-A of the tissue stabilizing member of FIG. 22B;

FIG. 23A is a perspective view of a housing assembly of the system for extracting hair follicles of FIG. 1A;

FIG. 23B is a side view of a first component of the housing assembly of FIG. 23A;

FIG. 23C is a side view of a second component of the housing assembly of FIG. 23A;

FIG. 24A is a top view of the systems of FIG. 1A;

FIG. 24B is a sectional view about the axis A-A of the systems of FIG. 24A;

FIG. 24C is a close-up view of a portion “P1” of the sectional view of FIG. 24B;

FIG. 24D is a close-up view of a portion “P2” of the sectional view of FIG. 24B;

FIG. 24E is a close-up view of a portion “P3” of the sectional view of FIG. 24B;

FIG. 24F is a close-up view of a portion “P4” of the sectional view of FIG. 24B;

FIGS. 24G-24Z 1 illustrates working of the system for extracting hair follicles;

FIG. 25A is a perspective view of the system for altering alignment of hair follicles;

FIG. 25B is another perspective view of the system of FIG. 25A;

FIG. 26 is a perspective view of a piercing member of the system of FIG. 25A;

FIG. 27A is a perspective view of an assembly in the system of FIG. 25A;

FIG. 27B is an exploded perspective view of the assembly of FIG. 27A;

FIG. 28 is a perspective view of a shaft of the assembly of FIG. 27A;

FIGS. 29A-29C are perspective views of an engagement member of the assembly of FIG. 27A;

FIG. 30 is a perspective view of the shaft and a compression member of the assembly of FIG. 27A;

FIG. 31A is a perspective view of a cap of the assembly of FIG. 27A;

FIG. 31B is a perspective view of teeth provided in the cap of FIG. 27A;

FIG. 32A is a perspective view of a second engagement member of the assembly of FIG. 27A;

FIG. 32B is a perspective view of teeth provided in the second engagement member of FIG. 32A;

FIG. 32C is a perspective view of second engagement member arms provided in the second engagement member of FIG. 32A;

FIG. 33 is a perspective view of a pivot enabling member of the assembly of FIG. 27A;

FIG. 34A is a front view of a guide member of the system of FIG. 25A;

FIG. 34B is a back view of the guide member of the system of FIG. 25A;

FIG. 34C is a perspective view of the guide member of the system of FIG. 25A;

FIG. 35A is a perspective view of an arm of the system of FIG. 25A;

FIG. 35B is another perspective view of the arm of the system of FIG. 25A;

FIG. 36A is a back view of a counter pressure device of the system of FIG. 25A;

FIG. 36B is a side view of the counter pressure device of the system of FIG. 25A;

FIG. 36C is a perspective view of latch of the arm of FIG. 35A;

FIG. 36D is a perspective view of an engagement screw of the system of FIG. 25A;

FIGS. 37A-37H are perspective views of parts of the assembly of FIG. 27A being engaged together;

FIGS. 38A-38F are perspective views illustrating the working of the assembly of FIG. 27A;

FIG. 39A is a perspective view of the arm and the assembly of the system of FIG. 25A;

FIG. 39B is another perspective view of the system of FIG. 25A;

FIG. 40A is a top view of the system of FIG. 25A;

FIG. 40B is a sectional perspective view of the system of FIG. 25A about the axis 40-40;

FIGS. 41A-41C are perspective views of the system of FIG. 25A;

FIGS. 42A-42F are perspective views illustrating altering an alignment and extraction of a hair follicle;

FIG. 43 is an exploded perspective view of an assembly;

FIG. 44 is a perspective view of a shaft of the assembly of FIG. 43;

FIG. 45 is a perspective view of an engagement member of the assembly of FIG. 43;

FIG. 46 is a perspective view of a cap of the assembly of FIG. 43;

FIG. 47 is an exploded perspective view of the cap, a second compression member and the shaft of the assembly of FIG. 43;

FIG. 47A is a perspective view of the shaft of the assembly of FIG. 43 adapted with a single piercing member;

FIG. 48 is a perspective view of a second engagement member of the assembly of FIG. 43;

FIG. 49 is a perspective view of an indexer of the assembly of FIG. 43;

FIG. 50 is a perspective view of a pivot enabling member of the assembly of FIG. 43;

FIG. 51 is a perspective view of a knob of the assembly of FIG. 43;

FIGS. 52A-52B are side view illustrating working of the knob of the assembly of FIG. 43;

FIGS. 53A-53H are perspective views of parts of the assembly of FIG. 43 being engaged together;

FIG. 531 is a side view of the assembly of FIG. 43;

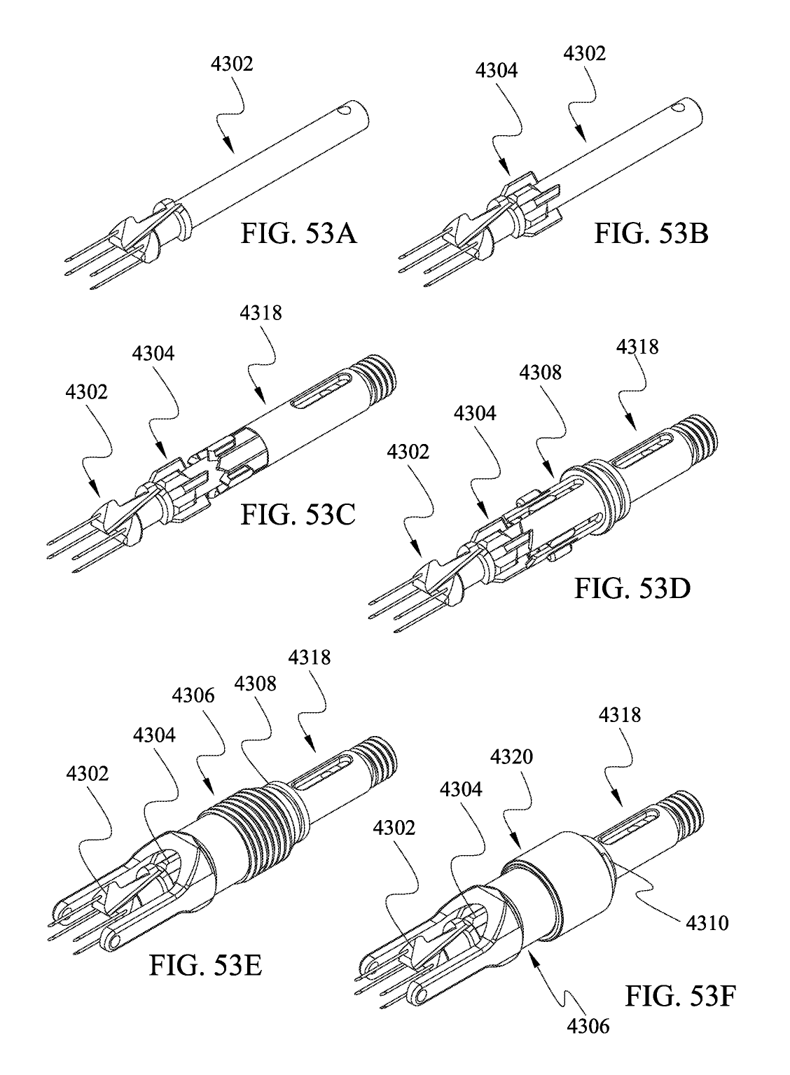

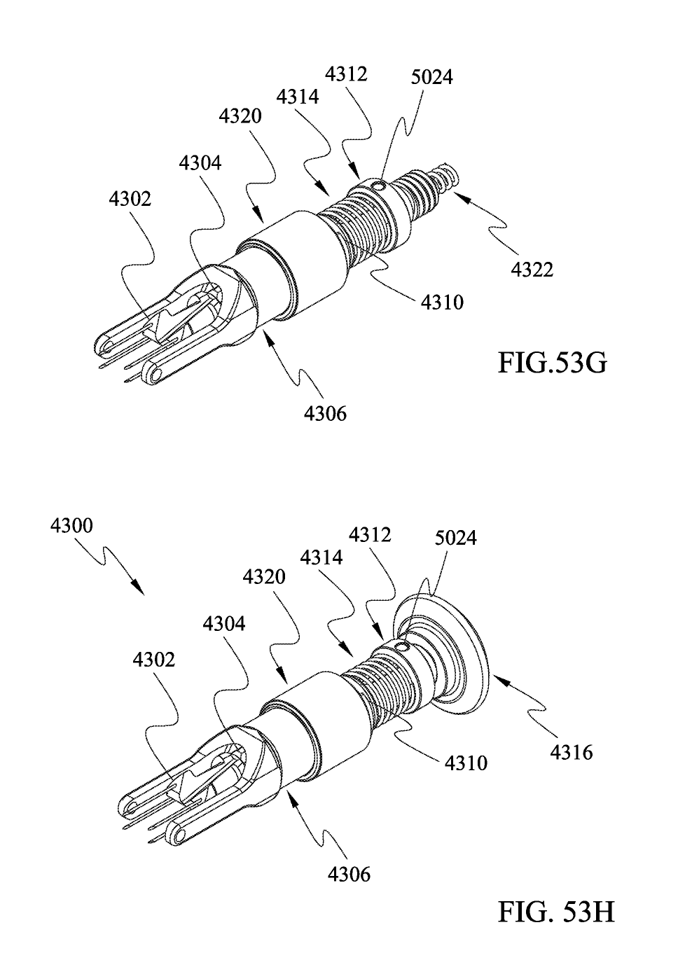

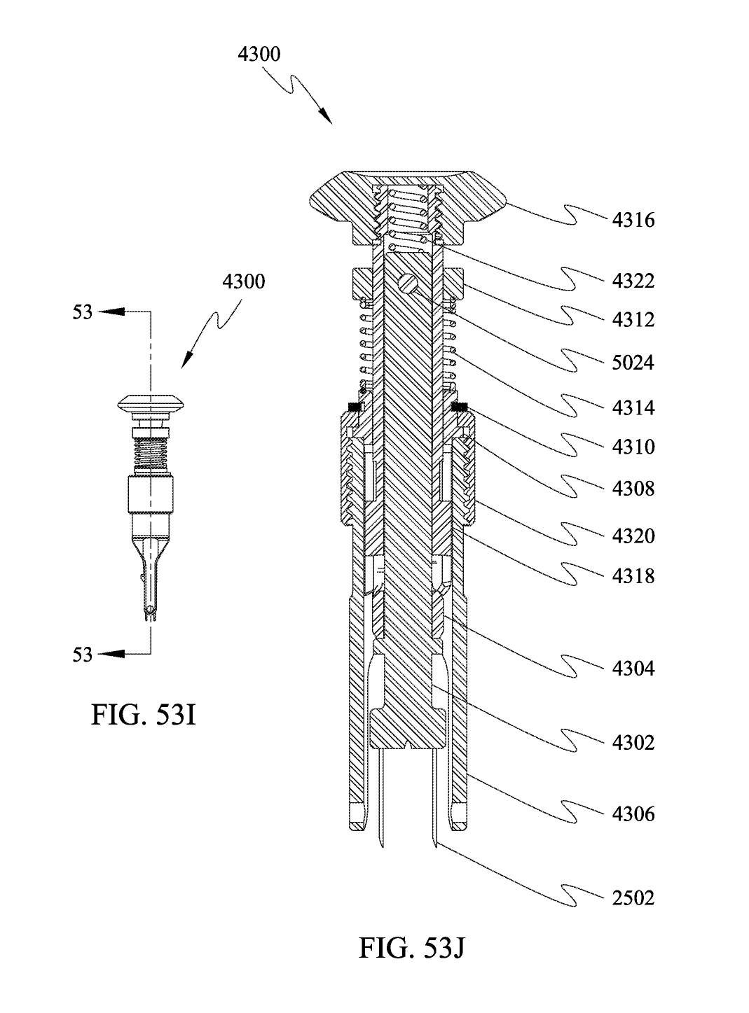

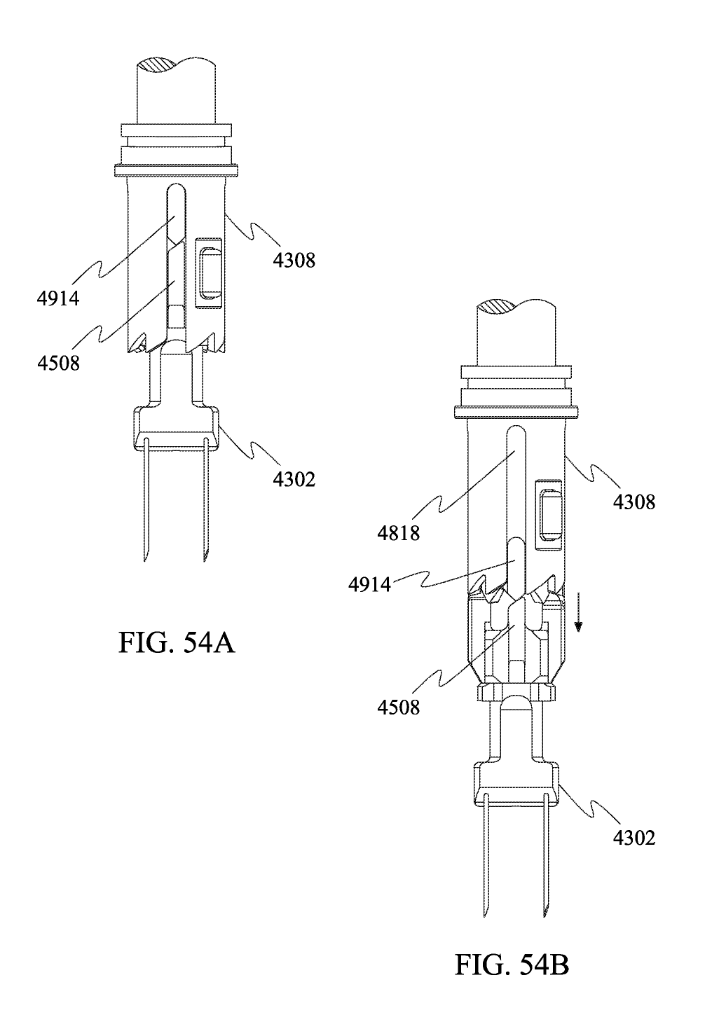

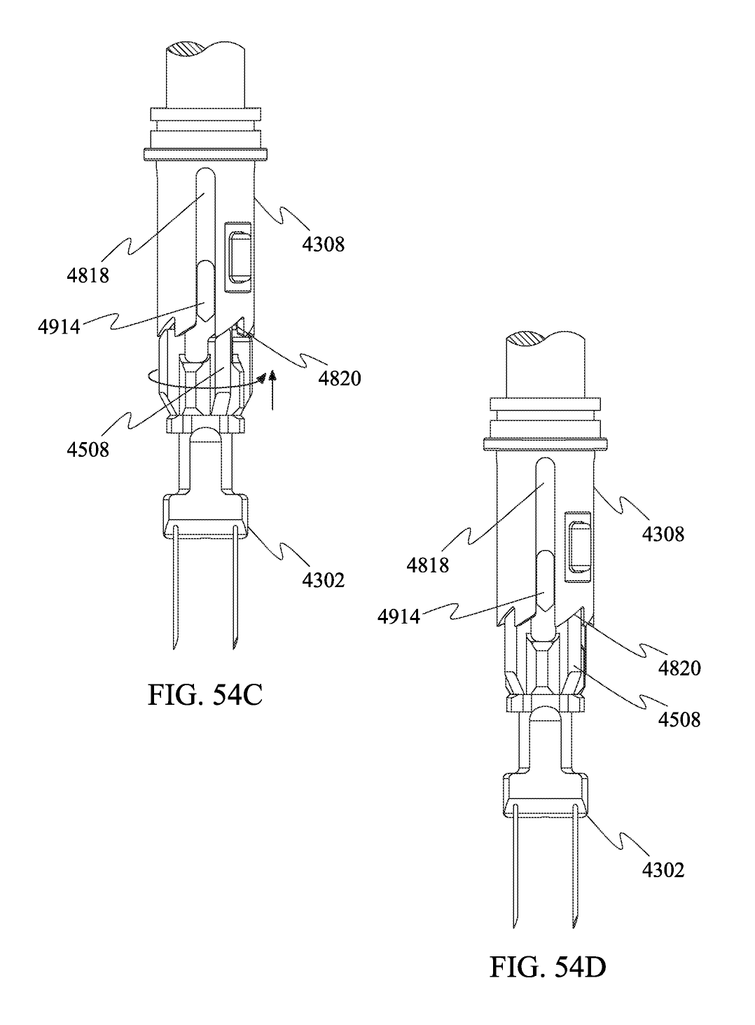

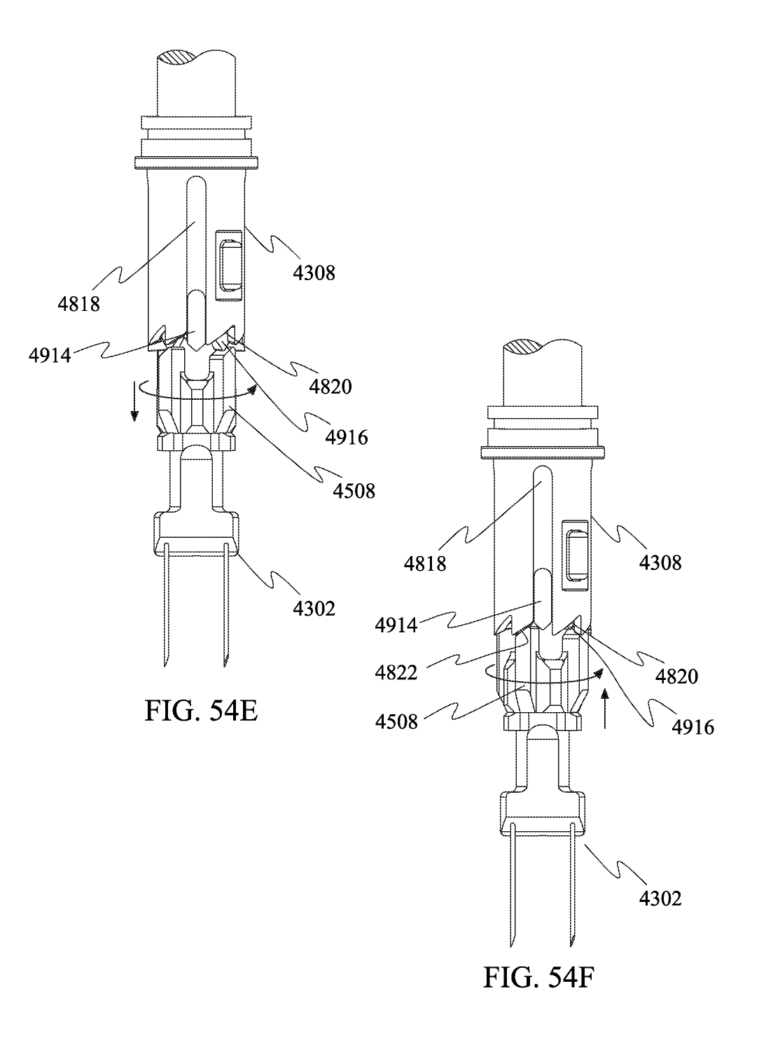

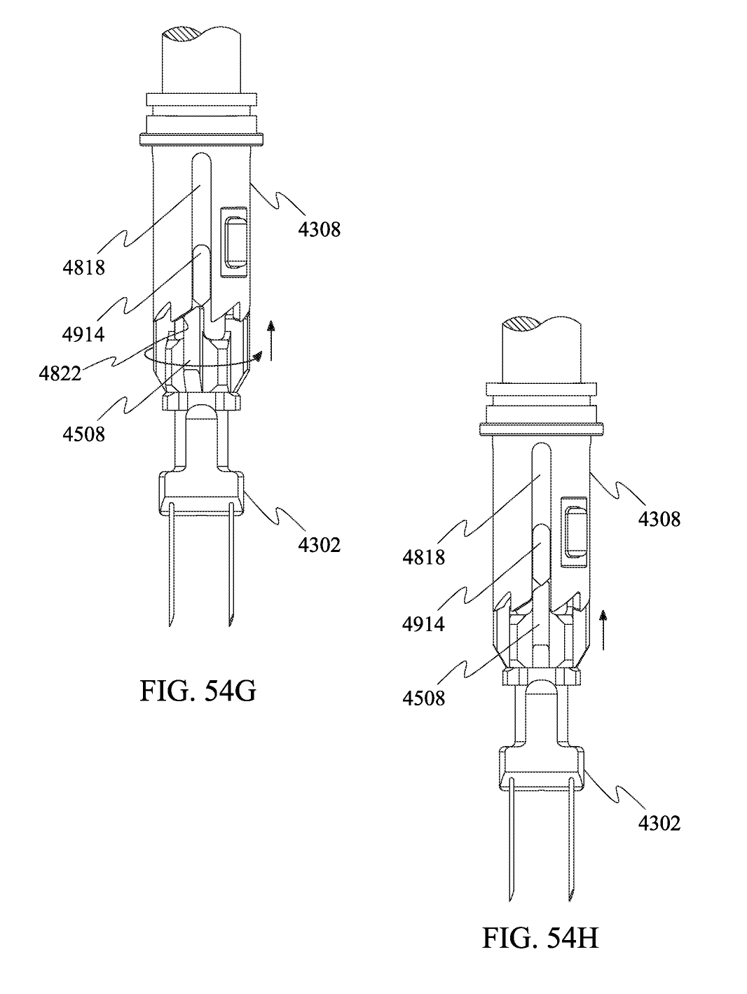

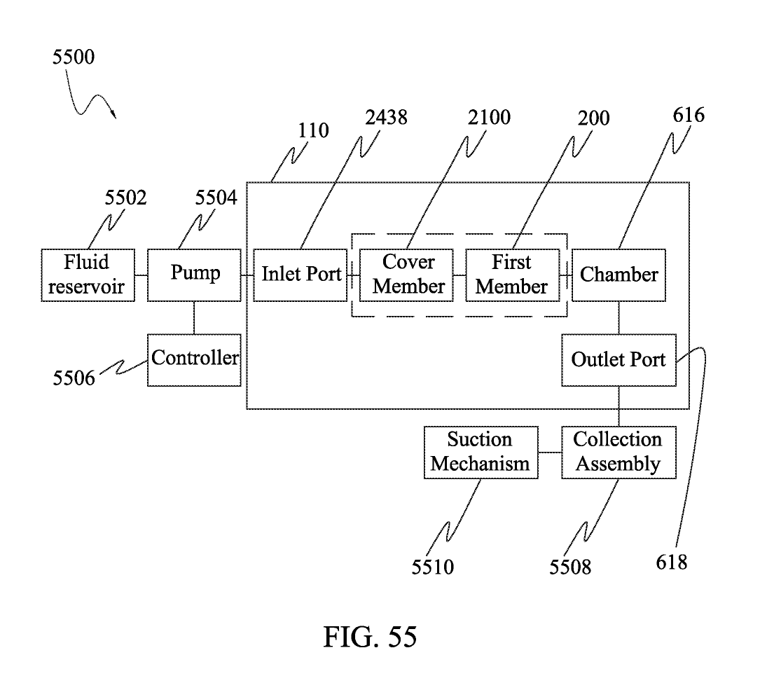

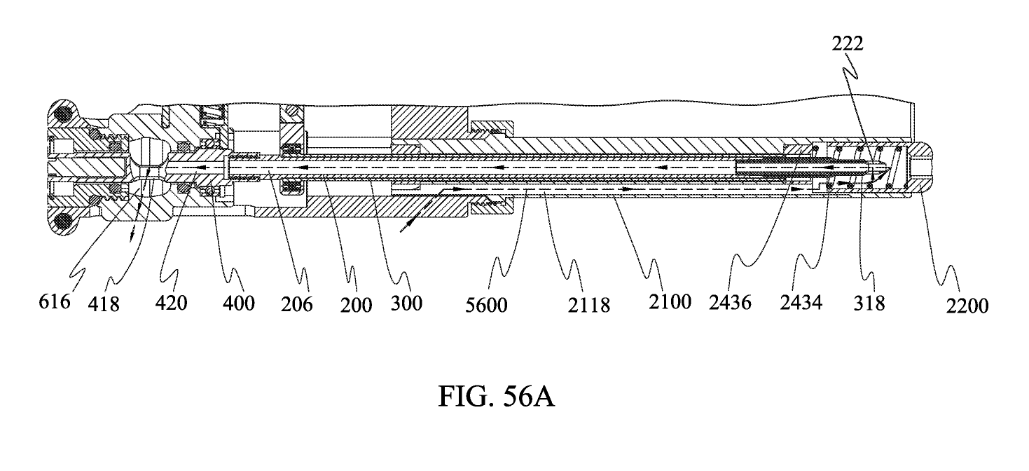

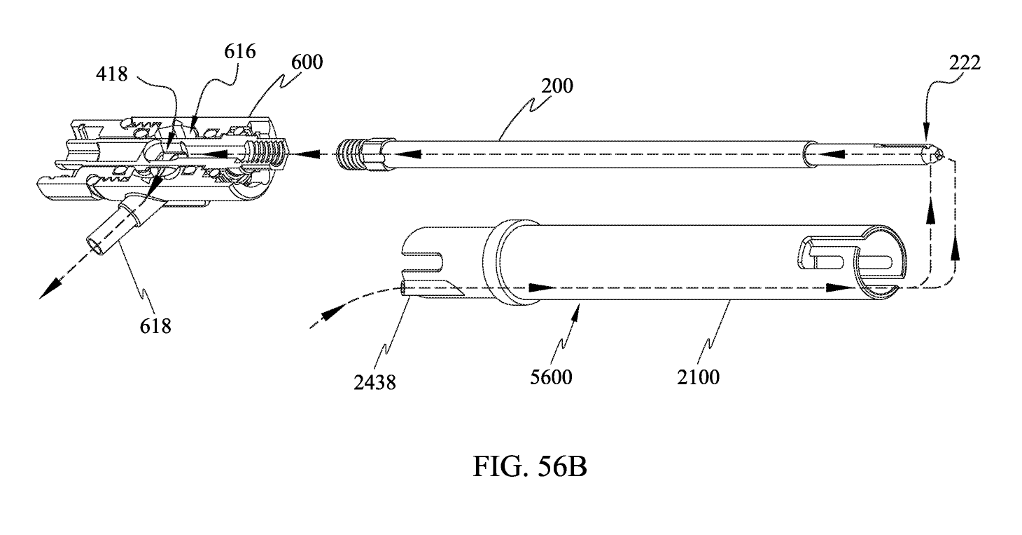

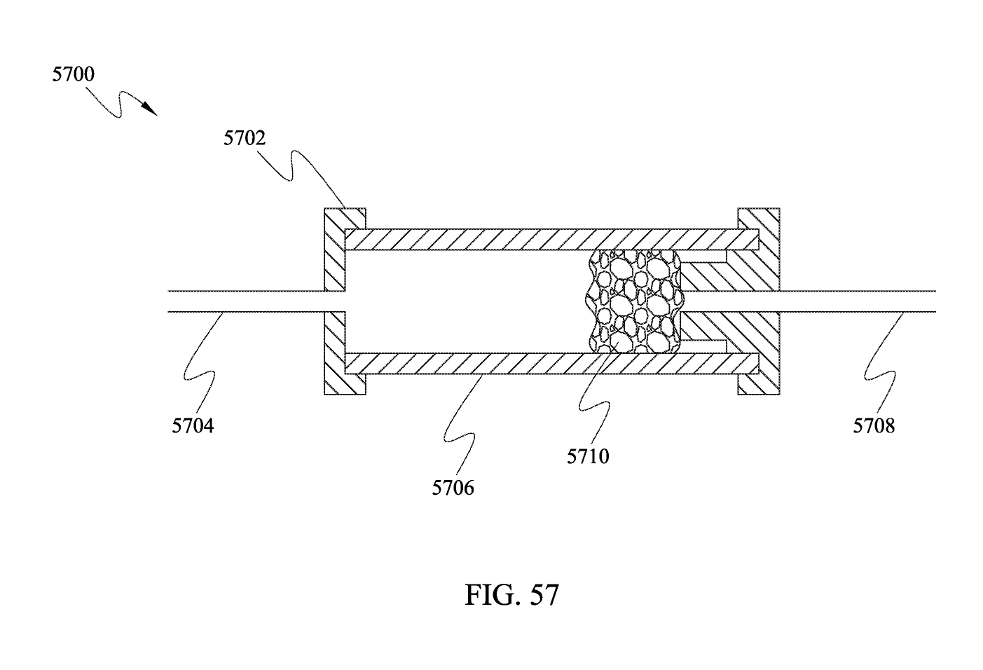

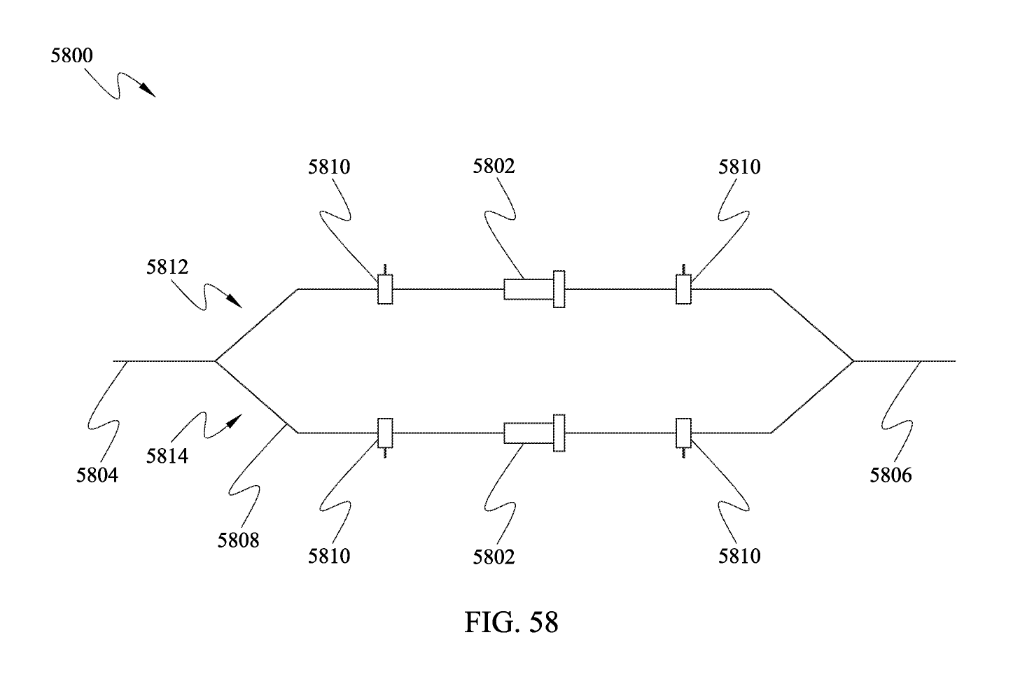

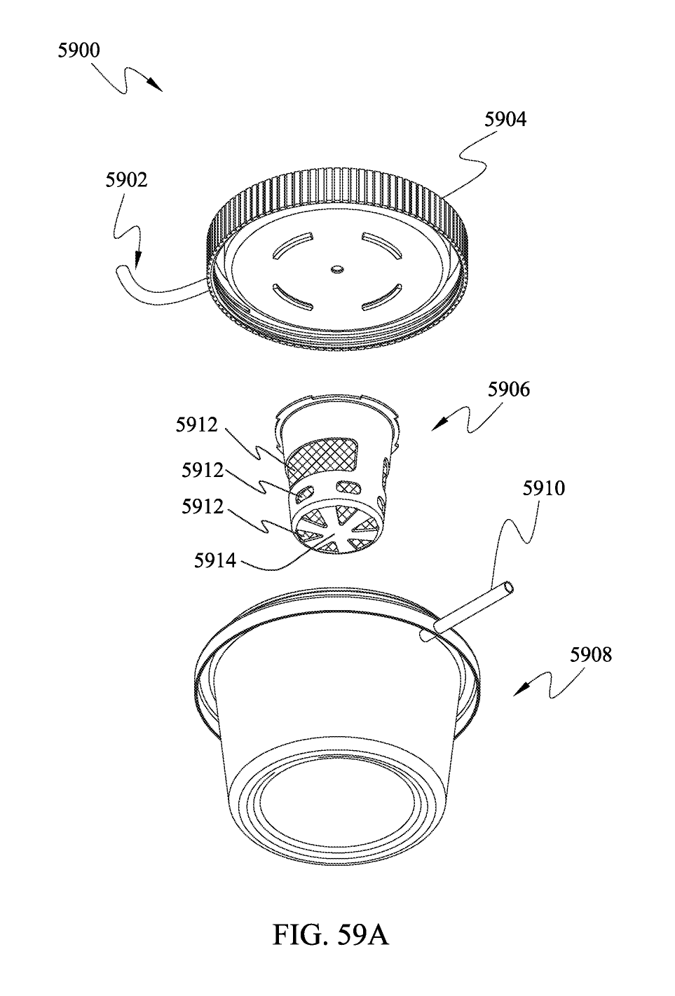

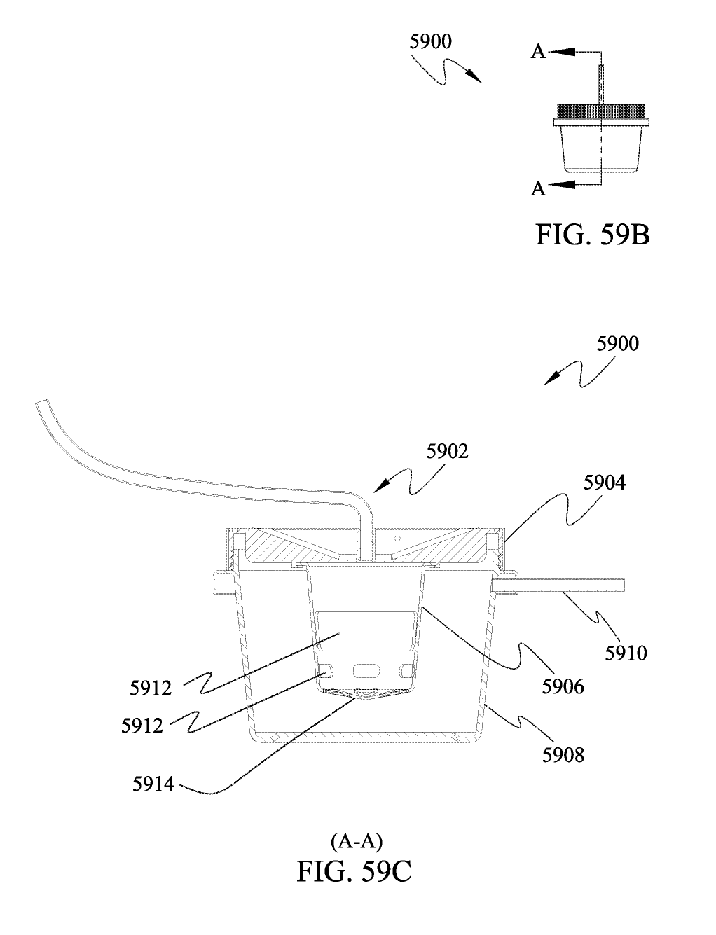

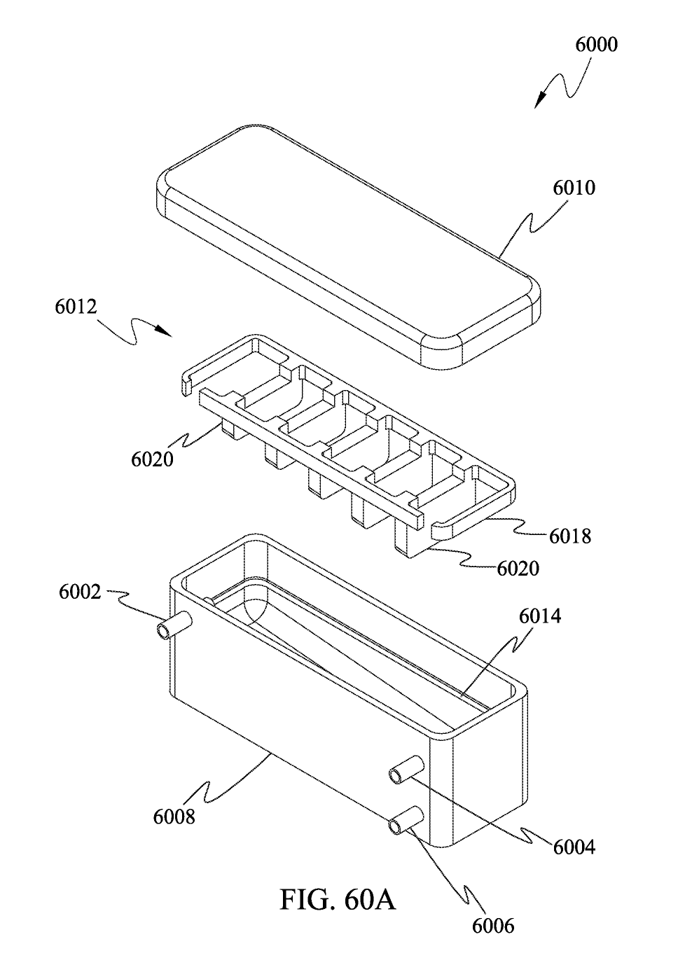

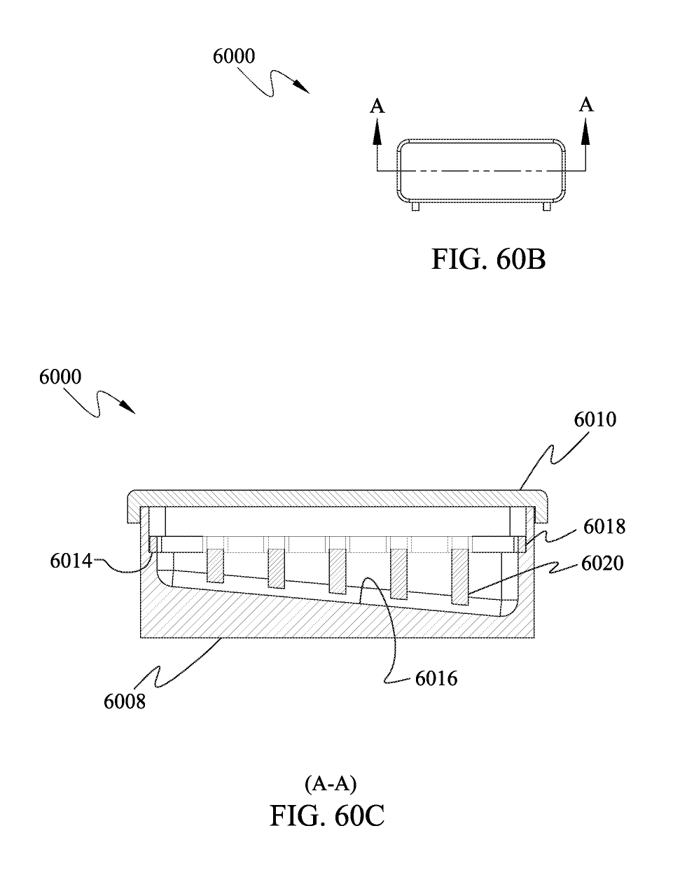

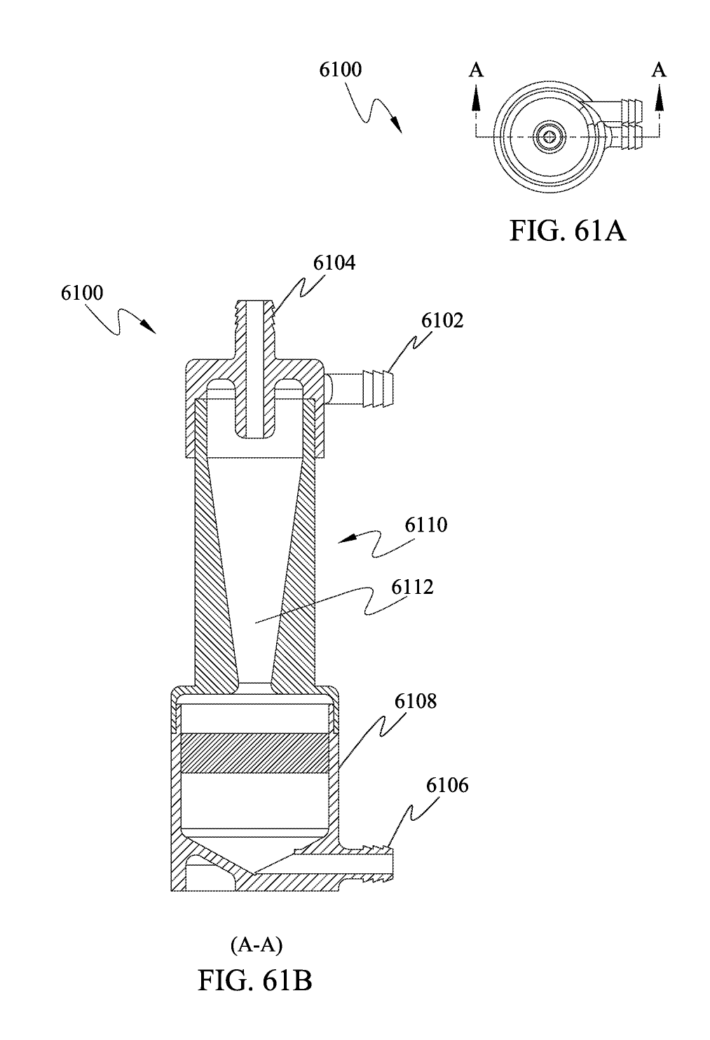

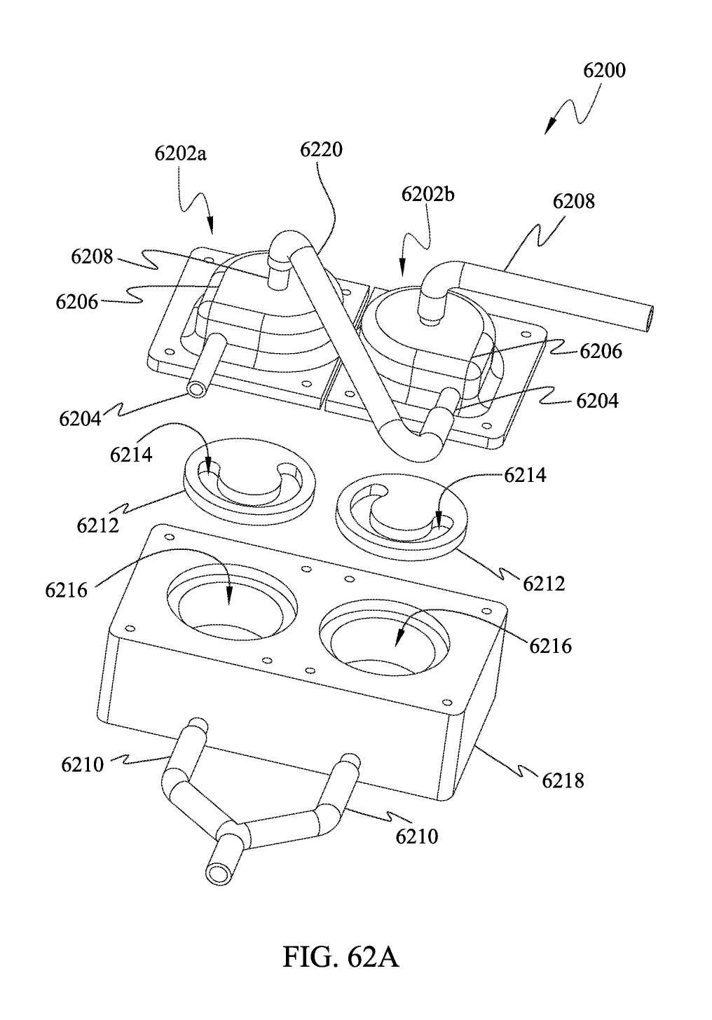

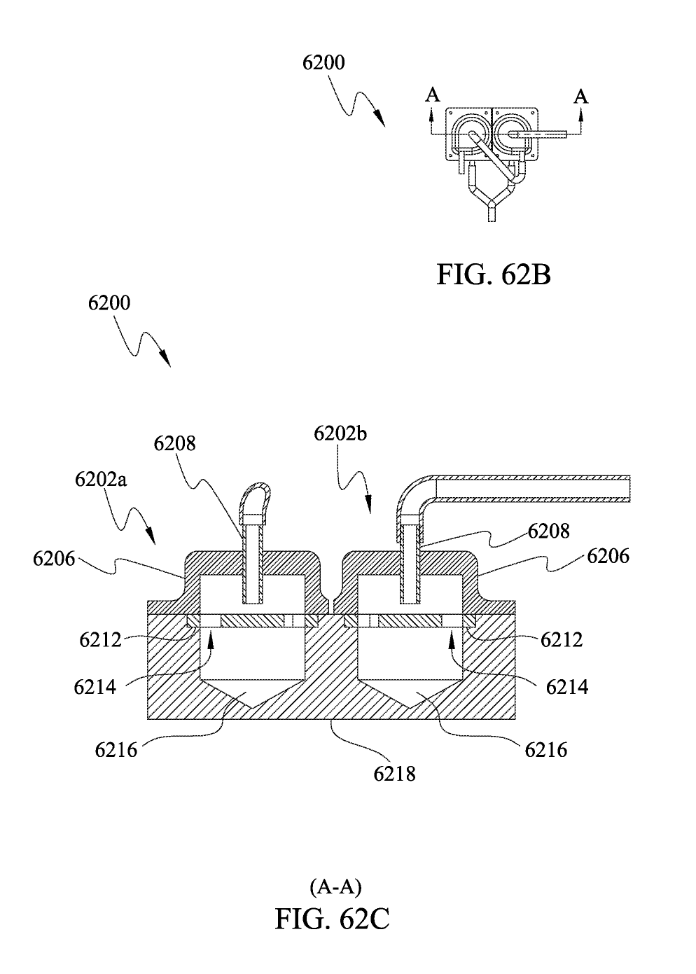

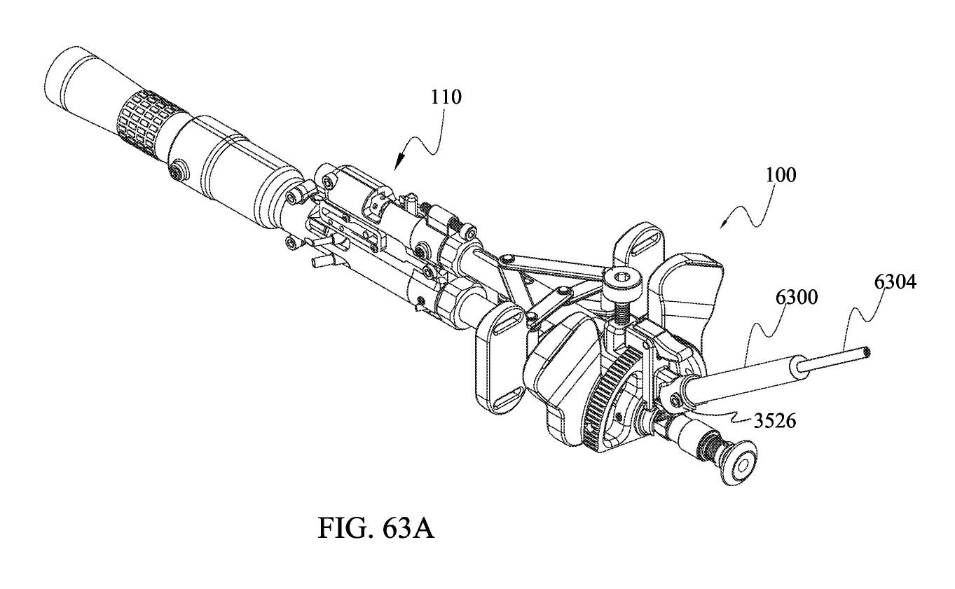

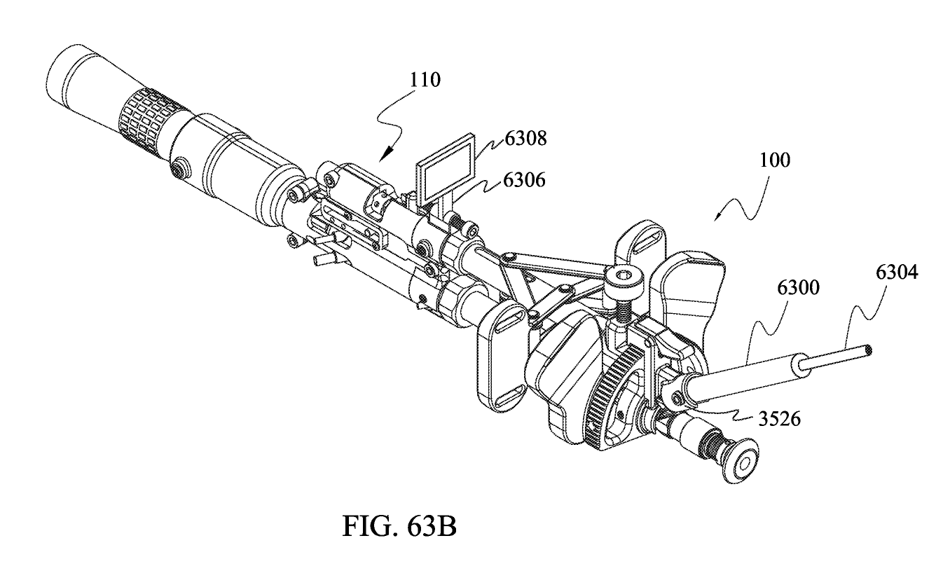

FIG. 53J is a sectional view of the assembly of FIG. 43 about the axis 53-53;

FIGS. 54A-54H are perspective views illustrating the working of the assembly of FIG. 43;

FIG. 55 is a schematic representation of a system for extracting tissue from underneath an external surface of the skin;

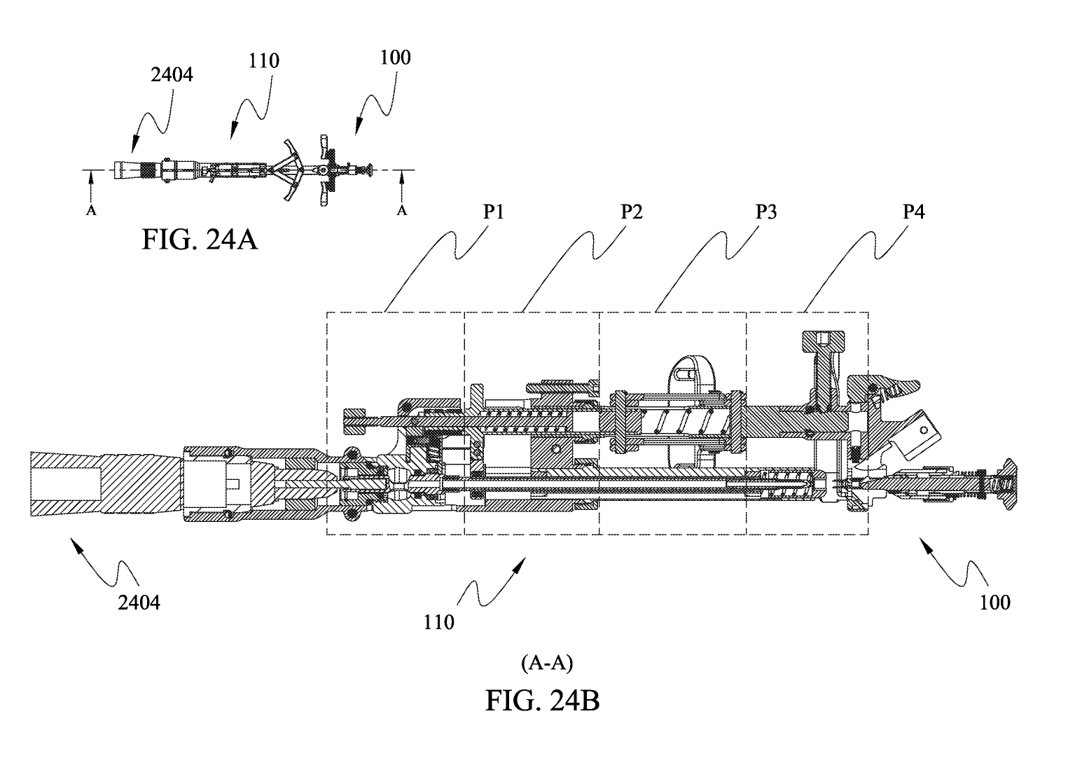

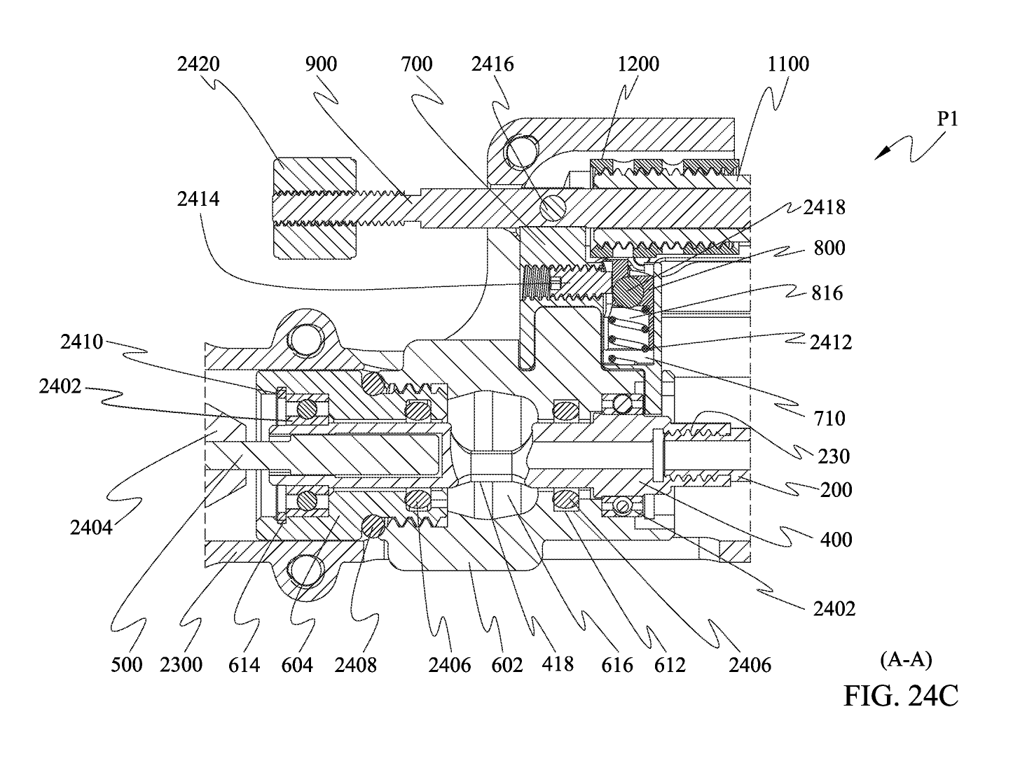

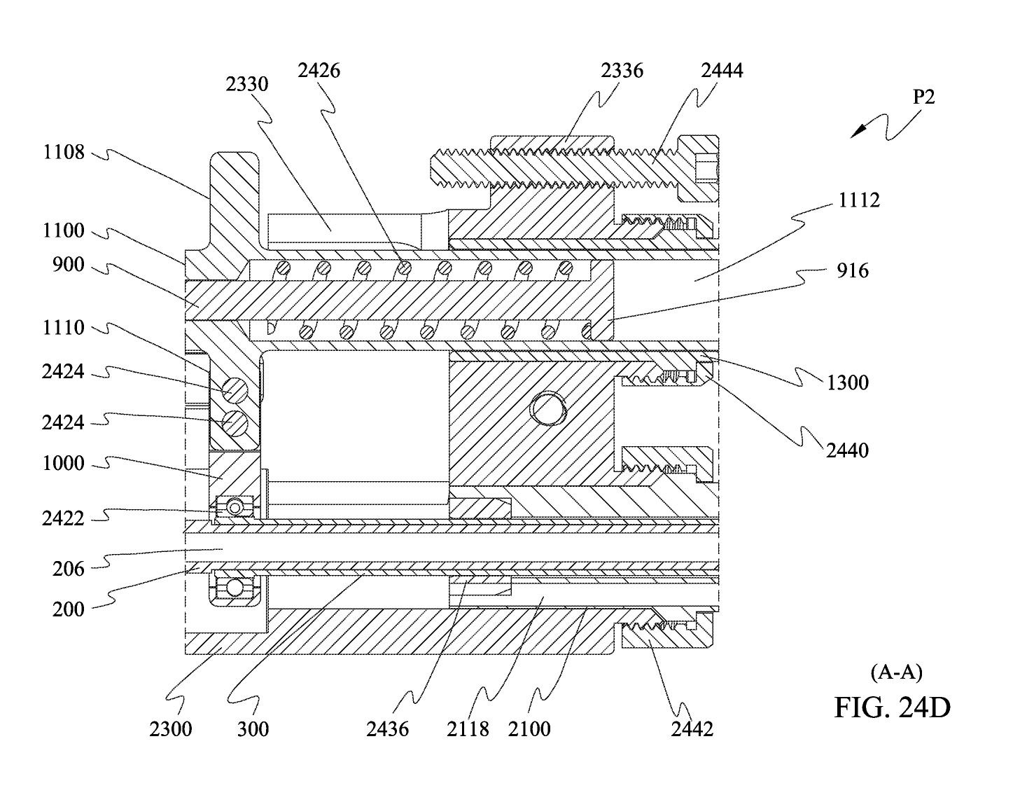

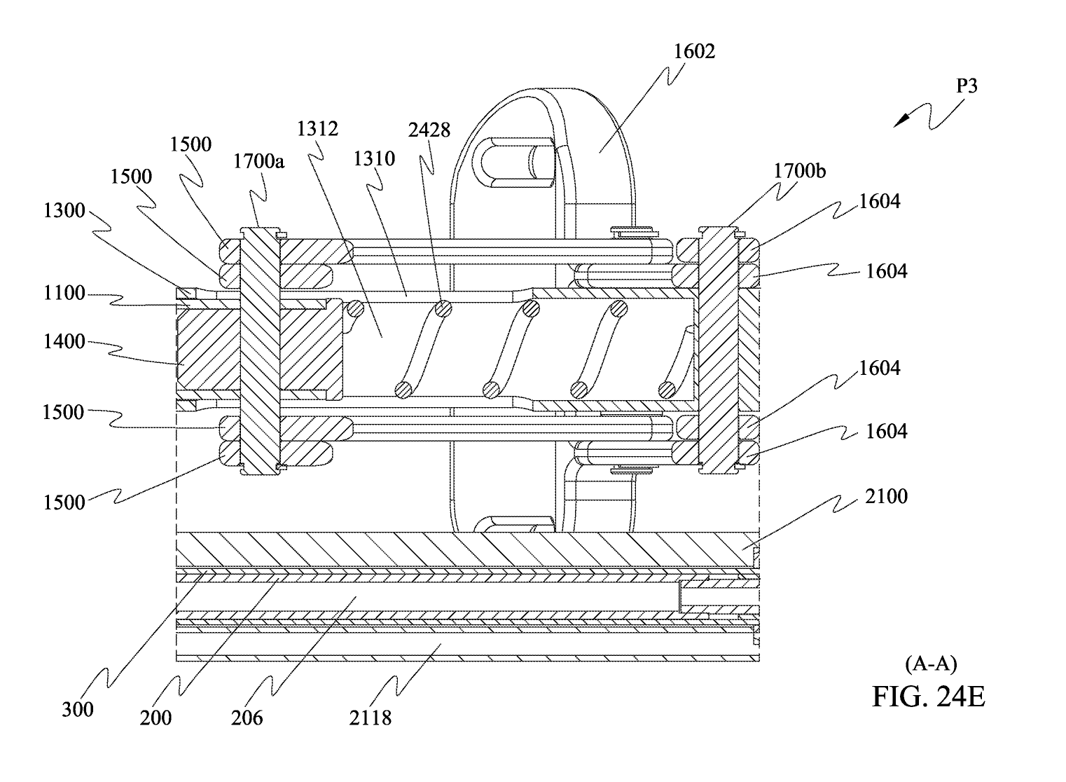

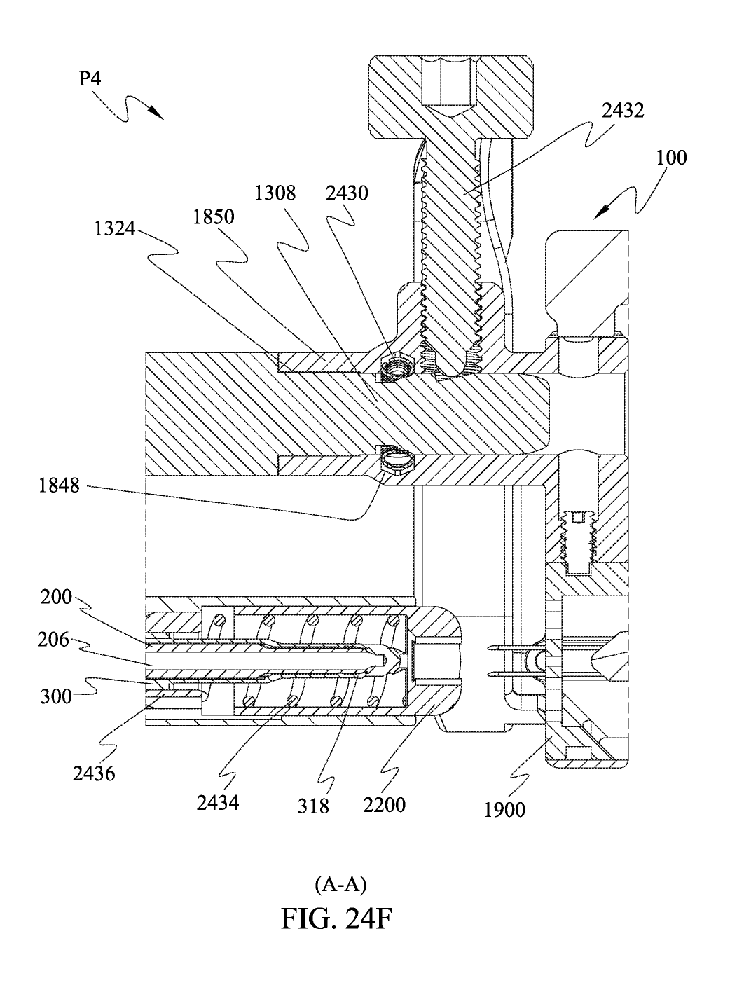

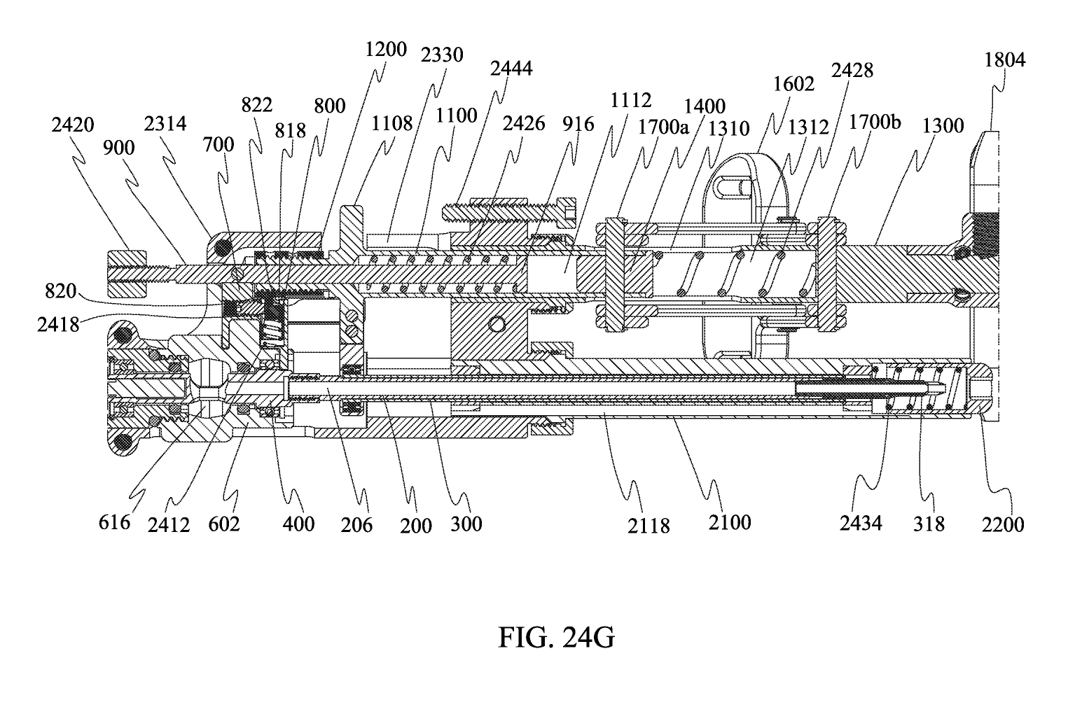

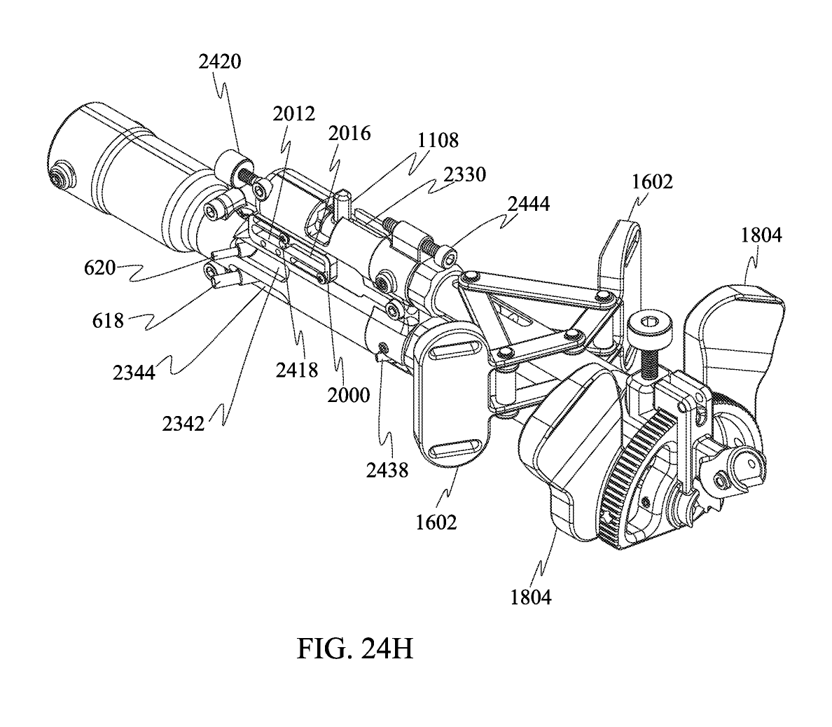

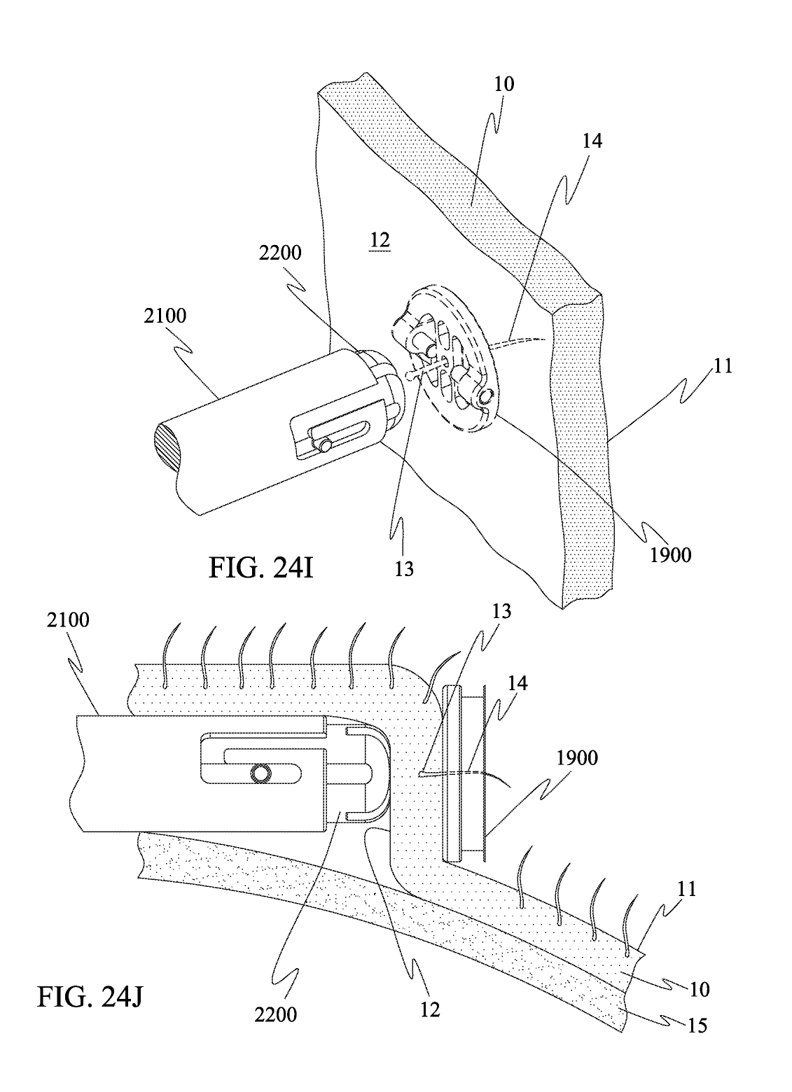

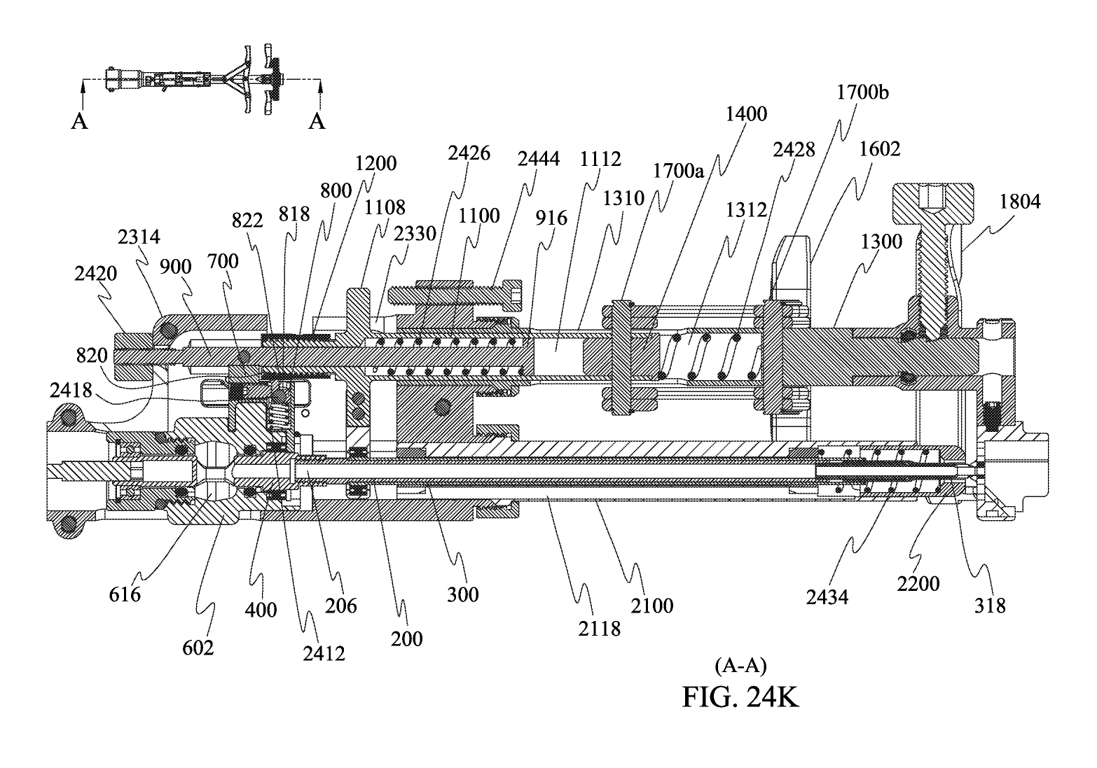

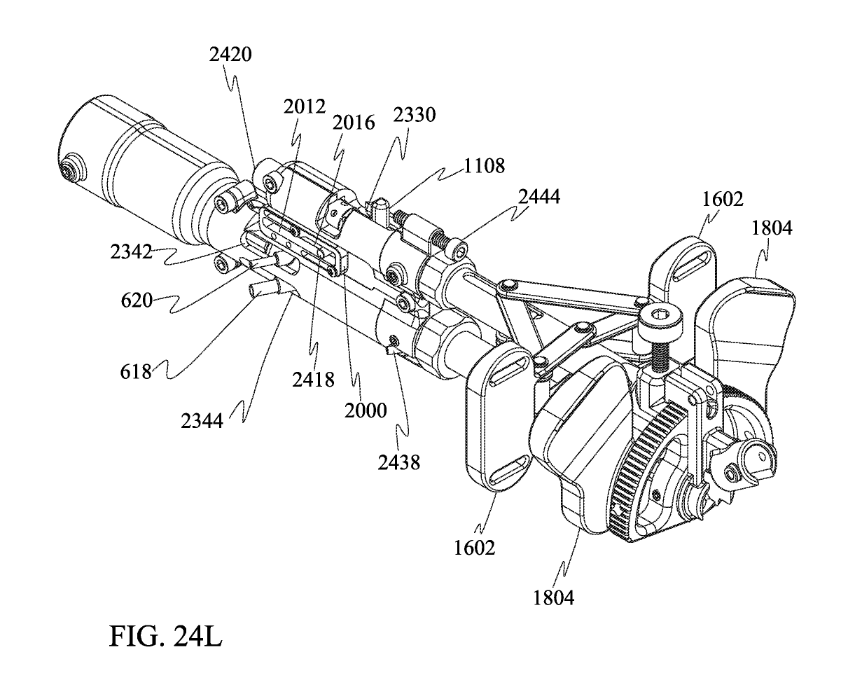

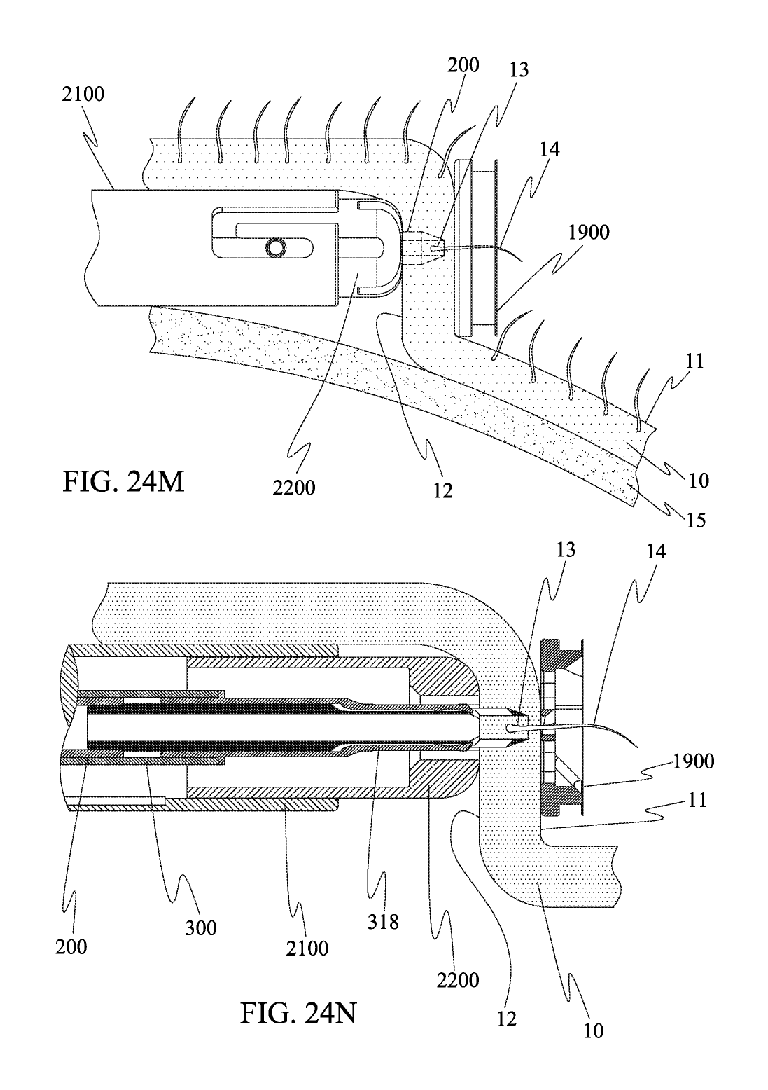

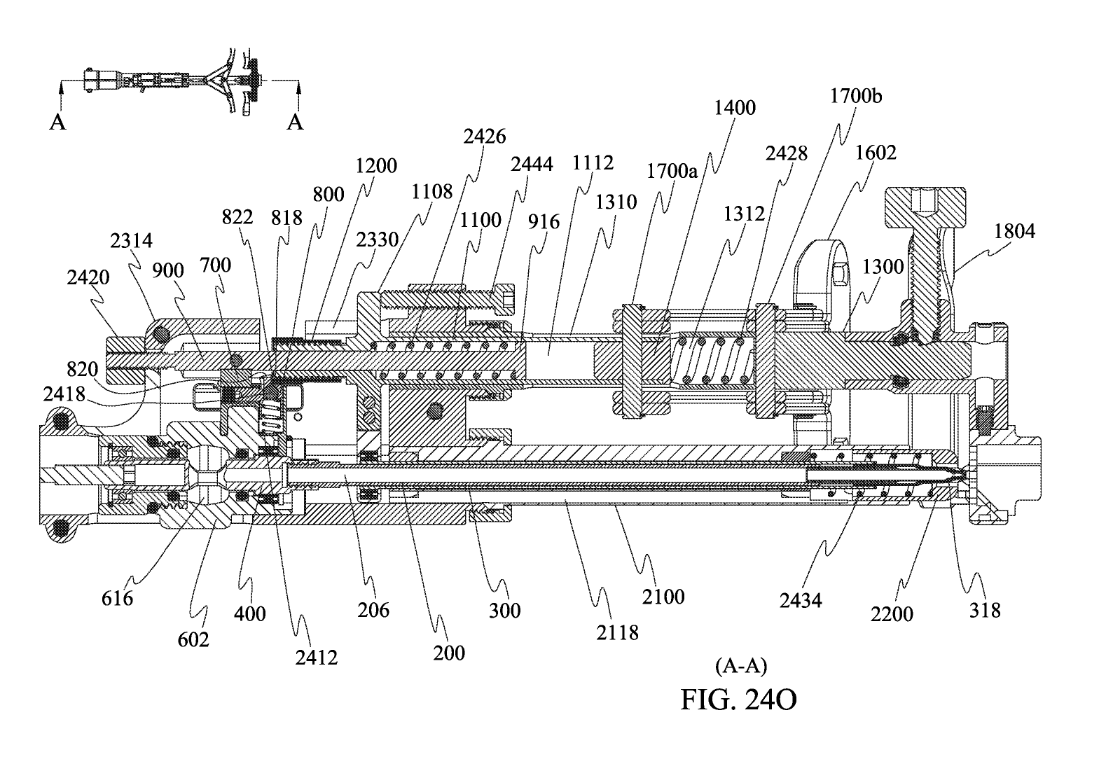

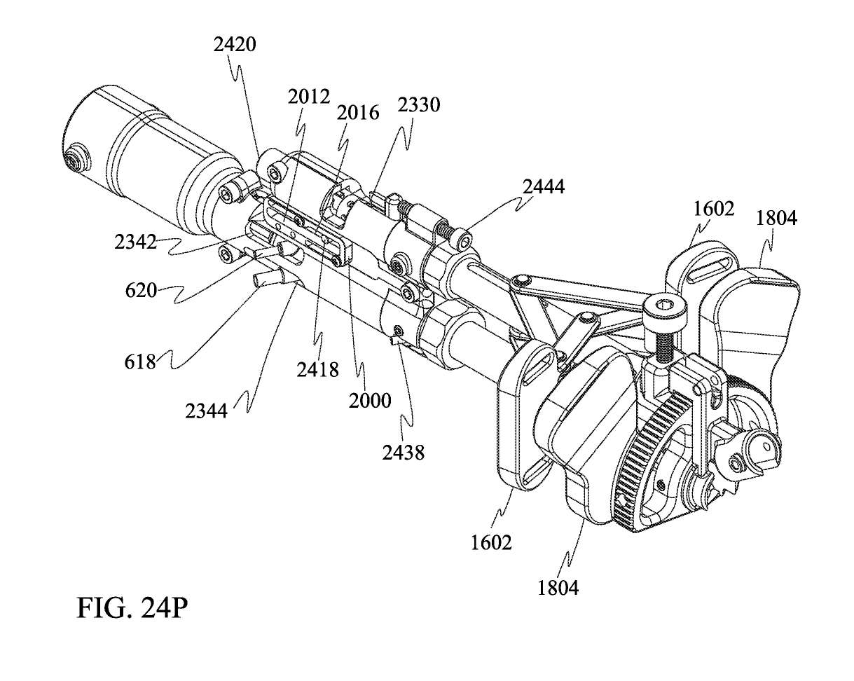

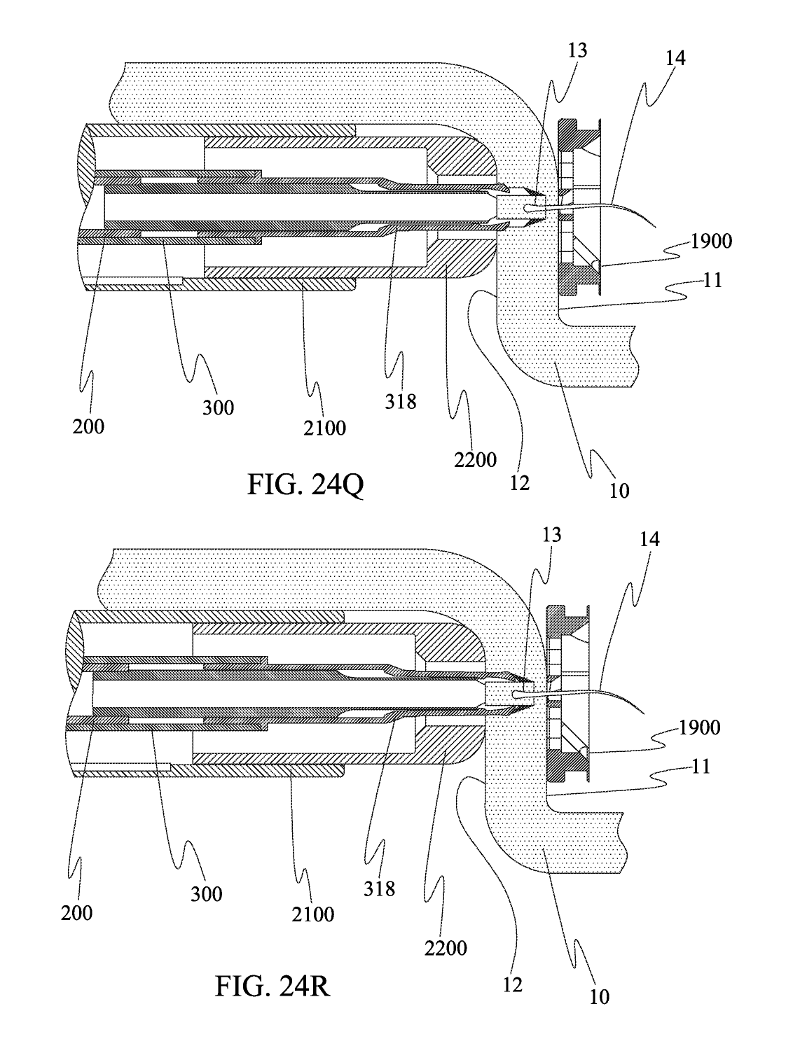

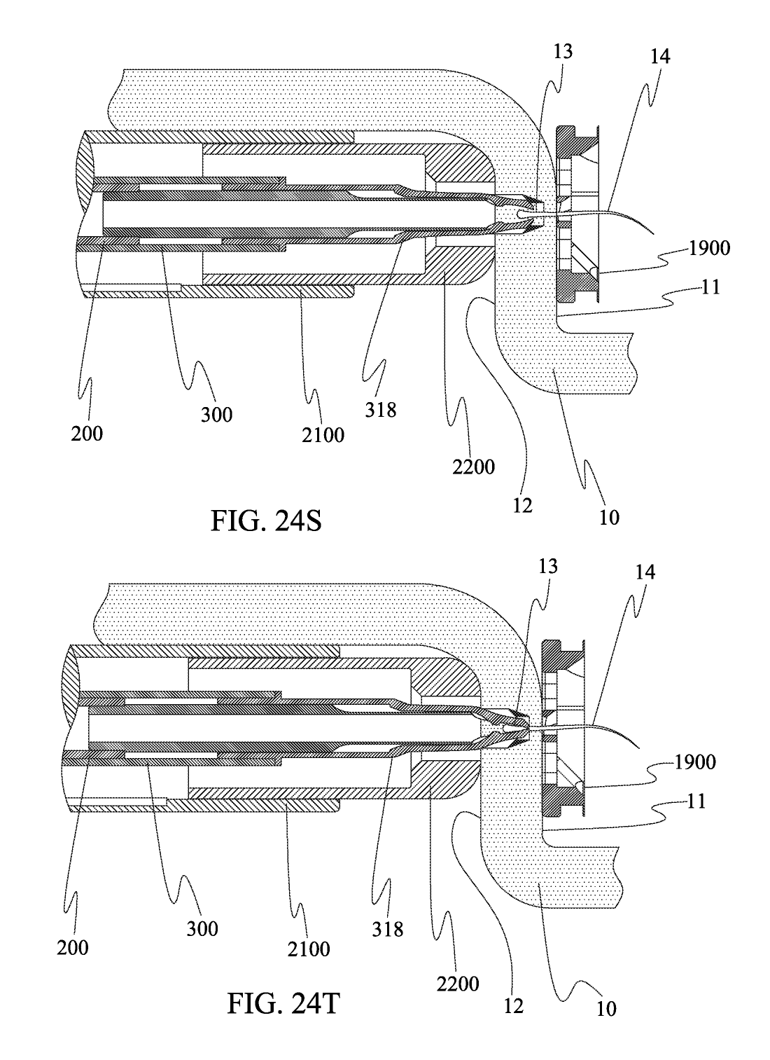

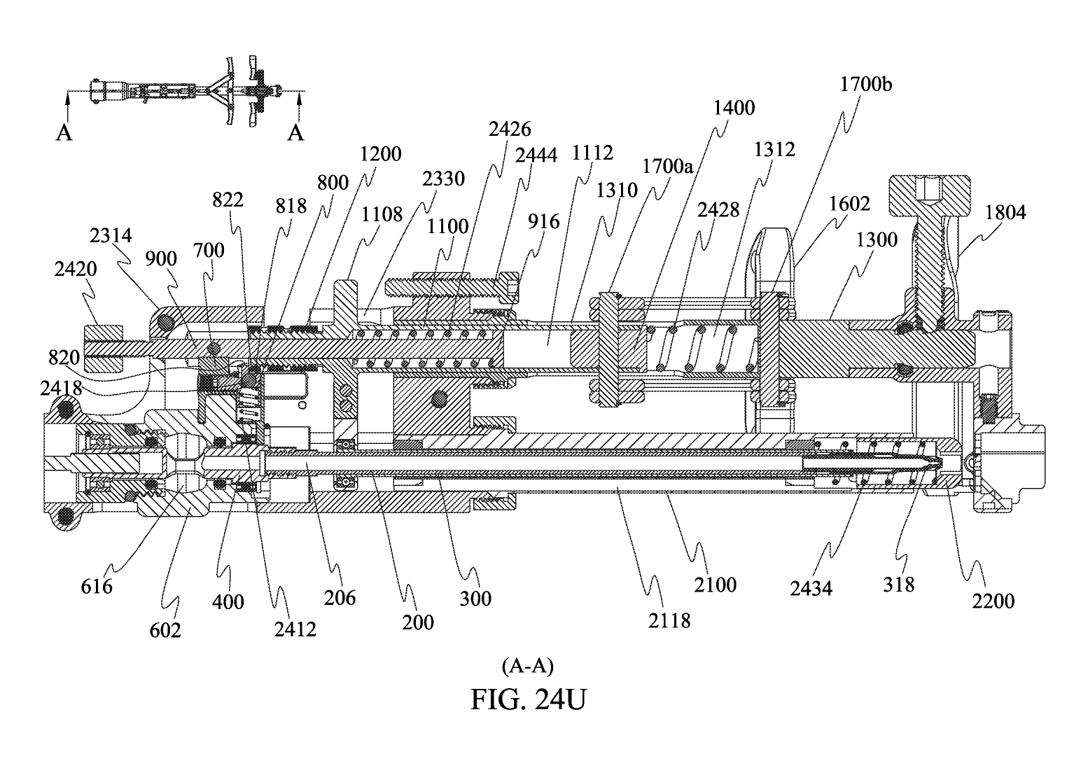

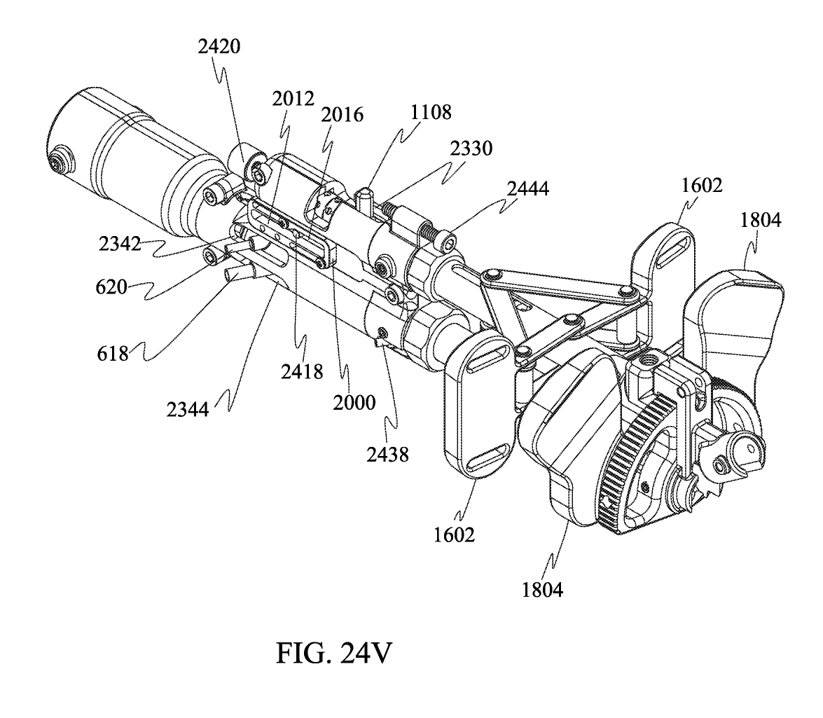

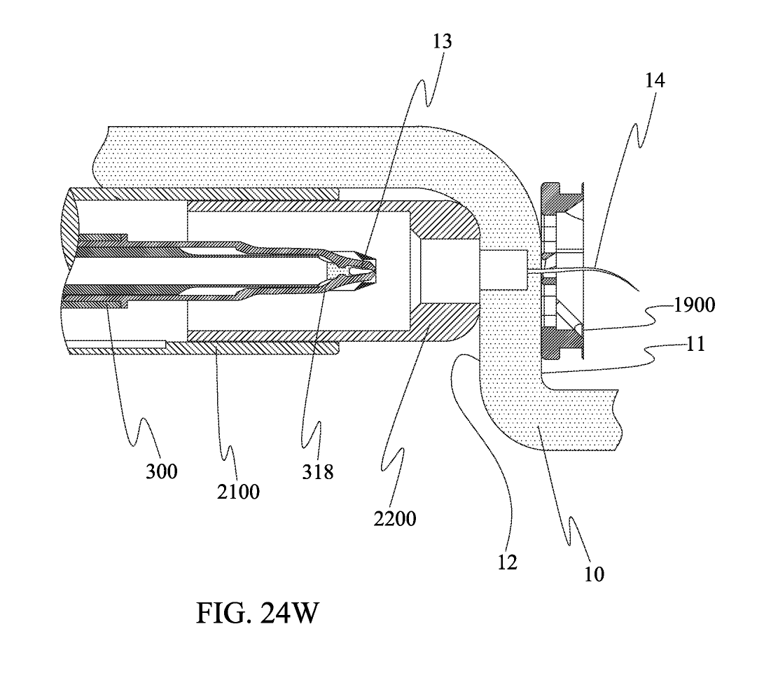

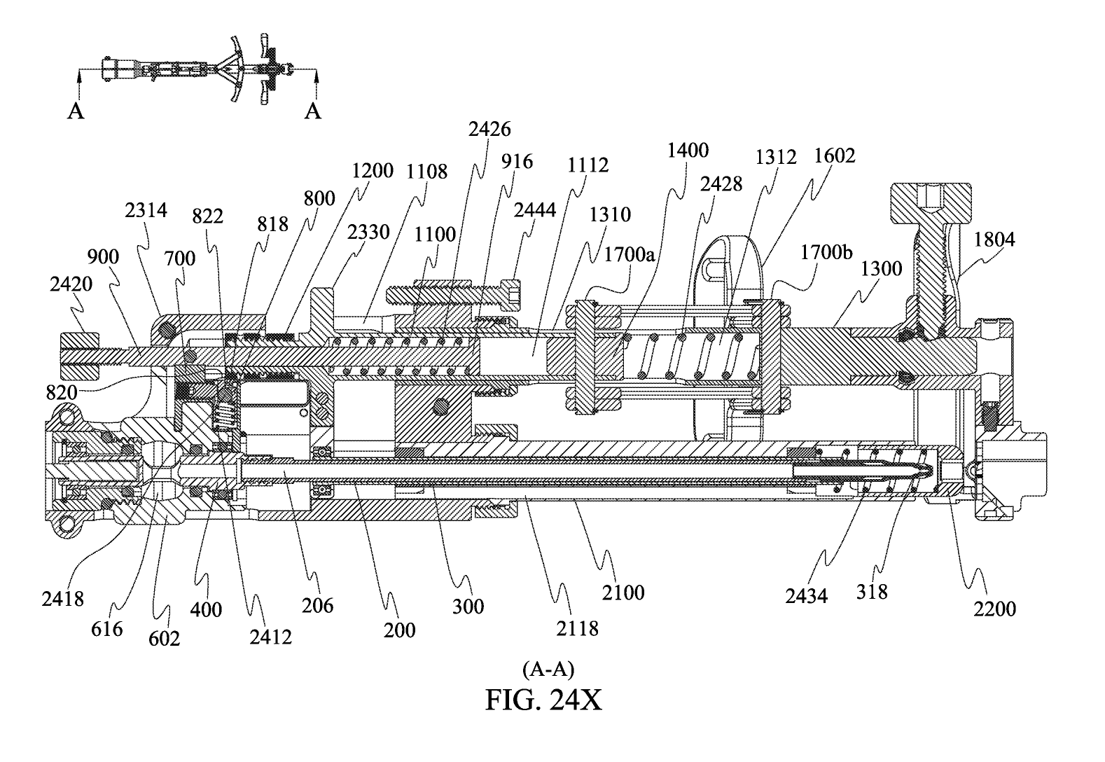

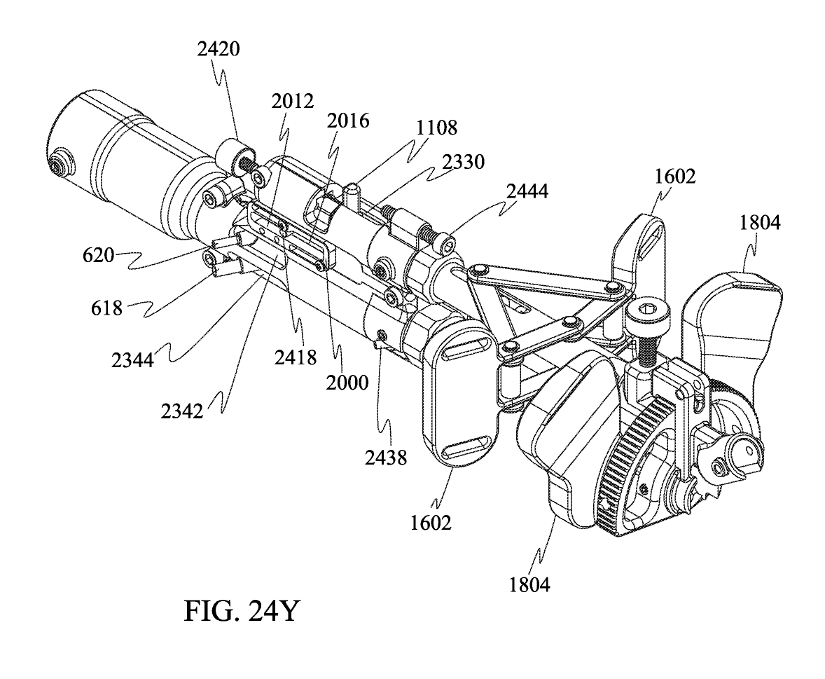

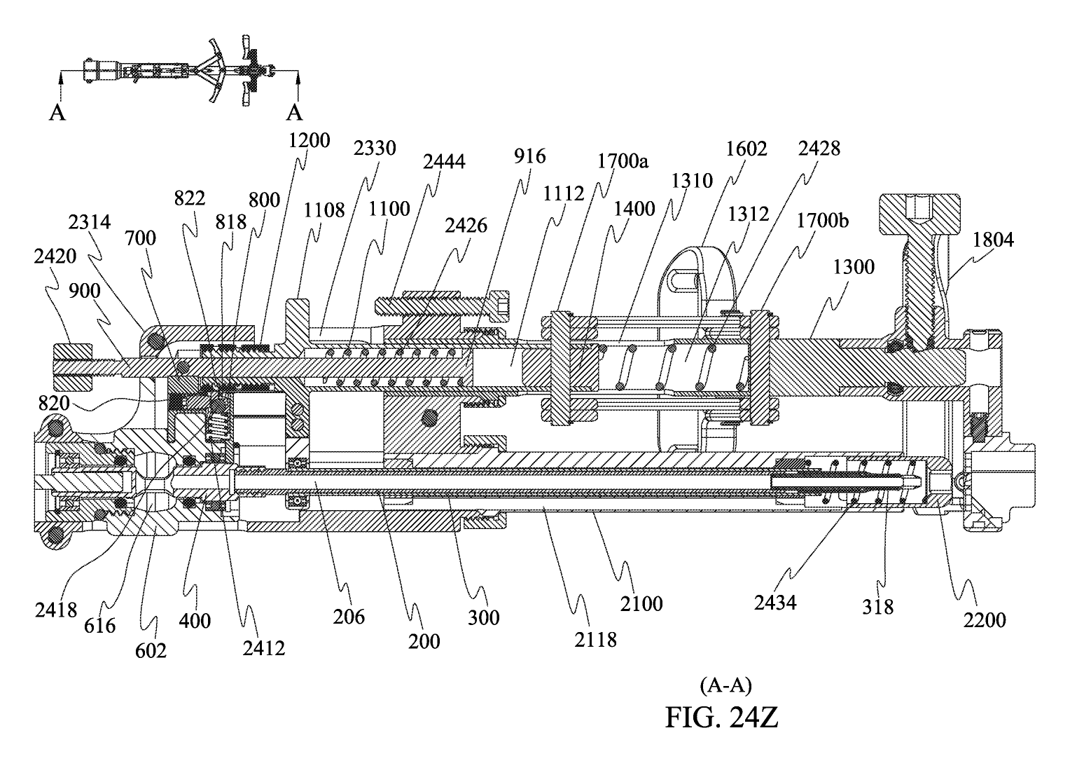

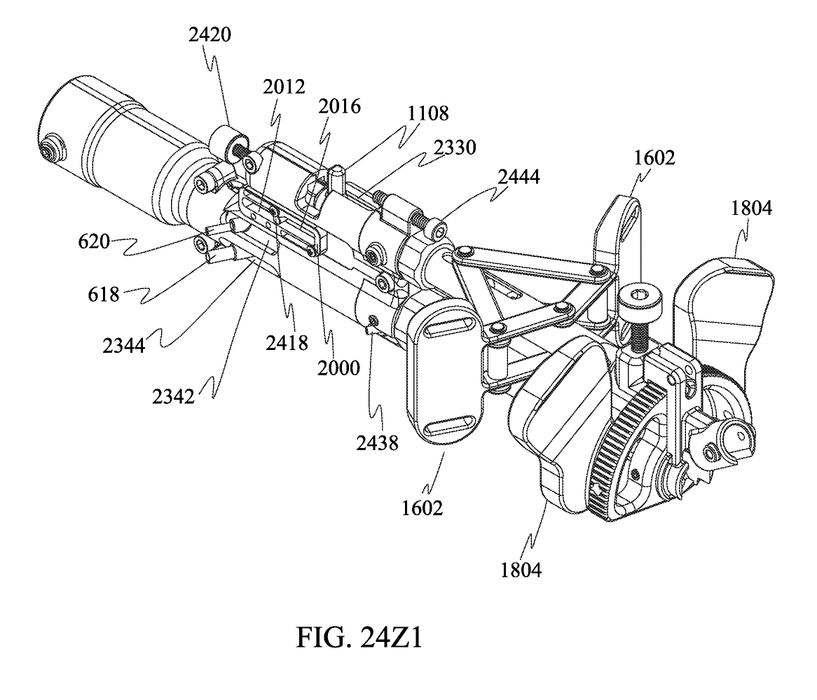

FIG. 56A is a portion of a sectional view about the axis A-A of the systems of FIG. 24A illustrating a first fluidic pathway;

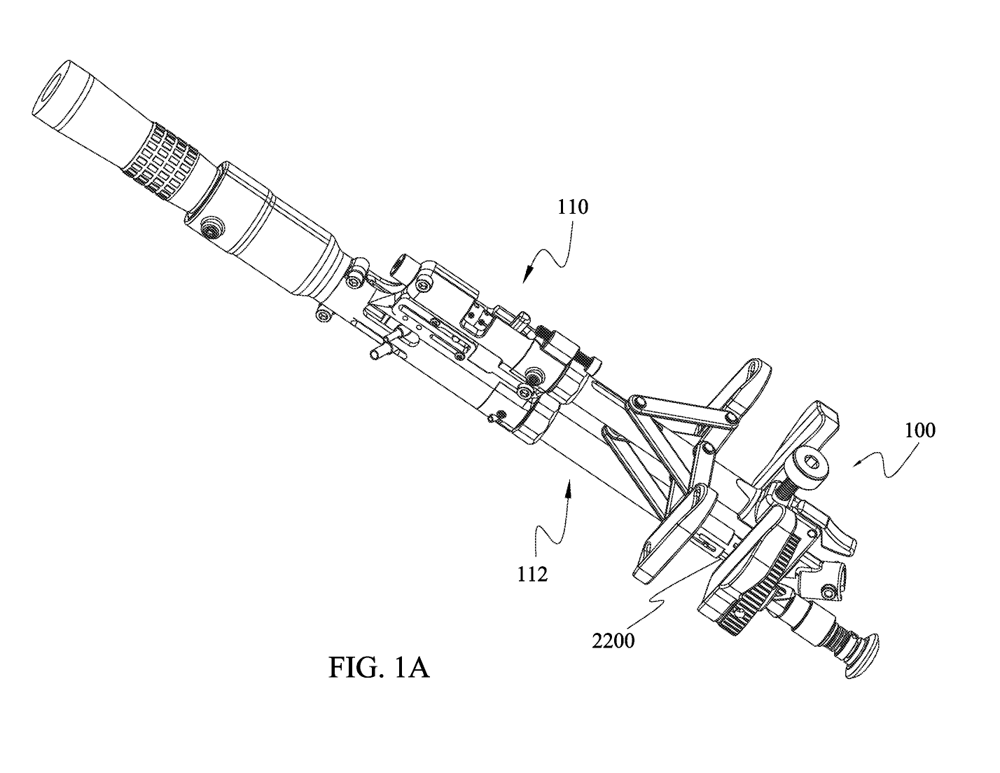

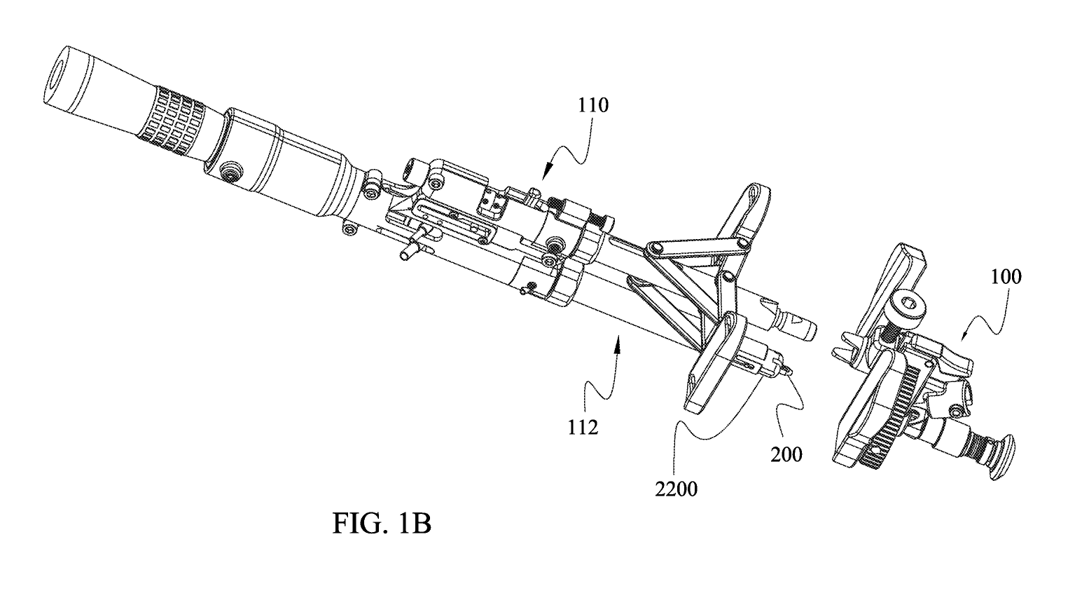

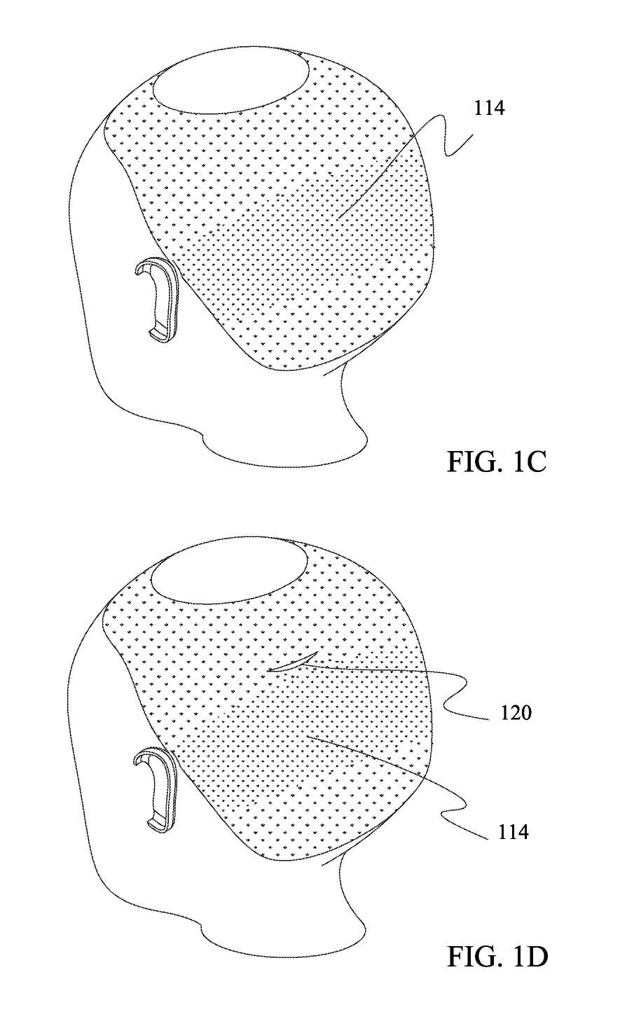

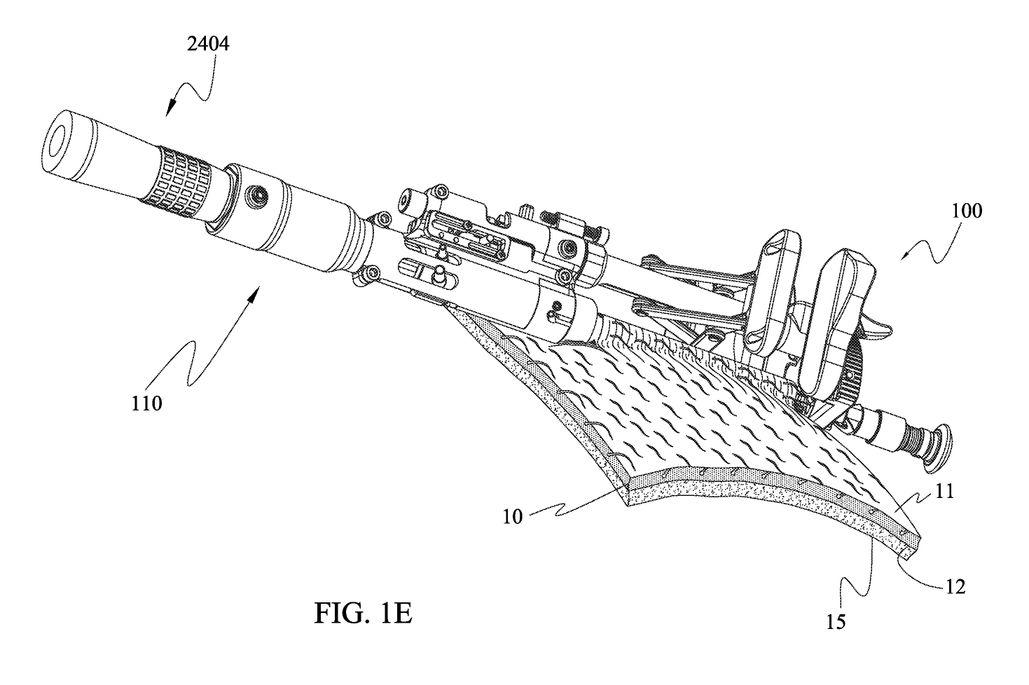

FIG. 56B is an exploded view of a few parts of the system of FIG. 1A illustrating the first fluidic pathway;

FIG. 57 is a schematic sectional view of a collection assembly;

FIG. 58 is a schematic view of another collection assembly;

FIG. 59A is a exploded perspective view of yet another collection assembly;

FIG. 59B is a side view of the collection assembly of FIG. 59A;

FIG. 59C is a sectional view about an axis A-A of the collection assembly of FIG. 59B;

FIG. 60A is a exploded perspective view of yet another collection assembly;

FIG. 60B is a top view of the collection assembly of FIG. 60A;

FIG. 60C is a sectional view about an axis A-A of the collection assembly of FIG. 60B;

FIG. 61A is a top view of yet another collection assembly;

FIG. 61B is a sectional view about an axis A-A of the collection assembly of FIG. 61A;

FIG. 62A is a exploded perspective view of yet another collection assembly;

FIG. 62B is a top view of the collection assembly of FIG. 62A;

FIG. 62C is a sectional view about an axis A-A of the collection assembly of FIG. 62B;

FIG. 63A is another perspective view of the systems of FIG. 1A; and

FIG. 63B is another perspective view of the systems of FIG. 1A.

### DETAILED DESCRIPTION

The disclosure may relate to extracting hair follicles from underneath an external surface of the skin, without punching holes through the external surface of the skin.

The following description illustrates principles, which may be applied in various ways to provide many different alternative embodiments. This description is not meant to limit the inventive concepts in the appended claims. The principles, structures, techniques, and methods disclosed herein may be adapted for use in other situations where a target tissue portion is to be extracted from a tissue region. For example, the present technology may be adapted for use in dermatology, cosmetic surgery, and/or general surgery. Although this disclosure focuses on extraction of head hair for subsequent transplantation, the disclosed technology also applies to extraction of hair follicles from other parts of the human body, such as the arm pit and pelvic regions, for the purpose of permanent hair removal. This technology also applies to extraction of other tissues, such as extraction of pathological tissues in the deeper layers of the skin or other body tissues, biopsy and/or removal of tissue being one example.

While exemplary embodiments of the present technology have been shown and described in detail below, it will be clear to the person skilled in the art that changes and modifications may be made without departing from its scope. As such, that which is set forth in the following description and accompanying drawings is offered by way of illustration only and not as a limitation. In addition, one of ordinary skill in the art will appreciate upon reading and understanding this disclosure that other variations for the technology described herein can be included within the scope of the present technology.

Referring to FIGS. 1A-1E, a device or system 110 may be used for extracting hair follicles from underneath an external surface 11 of the skin 10. The system 110 may be used with a system 100, which may be used for altering the alignment of hair follicles or tissue. The system 110 may be configured such that a portion 112 of the system 110 may be inserted under the skin 10 to access and/or cut through a tissue at a donor region 114 of the head. The system 110 may include a first member 200 which may be used to extract a portion of the hair follicle. The system 110 may further include a tissue stabilizing member 2200 which may be configured to apply pressure against or around a tissue comprising a hair follicle. The system 110 may be inserted into an area underneath the external surface 11 of the skin 10 by making an incision 120 on the external surface 11 of the skin 10 proximal or at the donor region 114. The first member 200 and the tissue stabilizing member 2200 may enter the area underneath the external surface 11 of the skin 10. The first member 200 may include a cutting edge configured to cut through the tissue. The system 100 may be positioned external to the overlying surface of the skin opposite to the first member 200. The system 100 may include a counter pressure device configured to interface with the external surface 11 of the skin 10, such that the tissue having the hair follicle is disposed between the counter pressure device and the tissue stabilizing member 2200, such that the tissue stabilizing member 2200 is disposed underneath the external surface 11 of the skin 10 and the counter pressure device is disposed over the external surface 11 of the skin 10.

The system for extracting hair follicles from underneath an external surface of the skin may include a first member and a second member. The first member and the second member may be configured to be moved underneath the skin. The first member may be a cylindrical shaft defining a first bore and having a cutting edge at its distal end. The second member may also be a cylindrical shaft configured to receive the first member. The second member may include a pair of arms towards its distal end. Both the first member and the second member may be configured to rotate about its longitudinal axis and also translate along its longitudinal axis. The system may include a tissue stabilizing member. The tissue stabilizing member may press around the tissue having the hair follicle from underneath the skin. The first member may translate towards the tissue while being rotated. As the first member translates into the tissue, the tissue is cut or cored by the cutting edge of the first member, and may create a channel in which the cut or cored tissue may be disposed. Subsequently, the second member may be translated relative to the first member. The relative translation of the second member may enable the arms of the second member to extend into the first bore of the first member through apertures provided on the cylindrical body of the first member close to its distal end. The arms of the second member may meet inside the first bore of the first member, thereby clipping the tissue that has been cut or cored by the first member. The tissue or the hair follicle may be aligned, for example by the system 100, with the longitudinal axis of the first member before cutting the tissue.

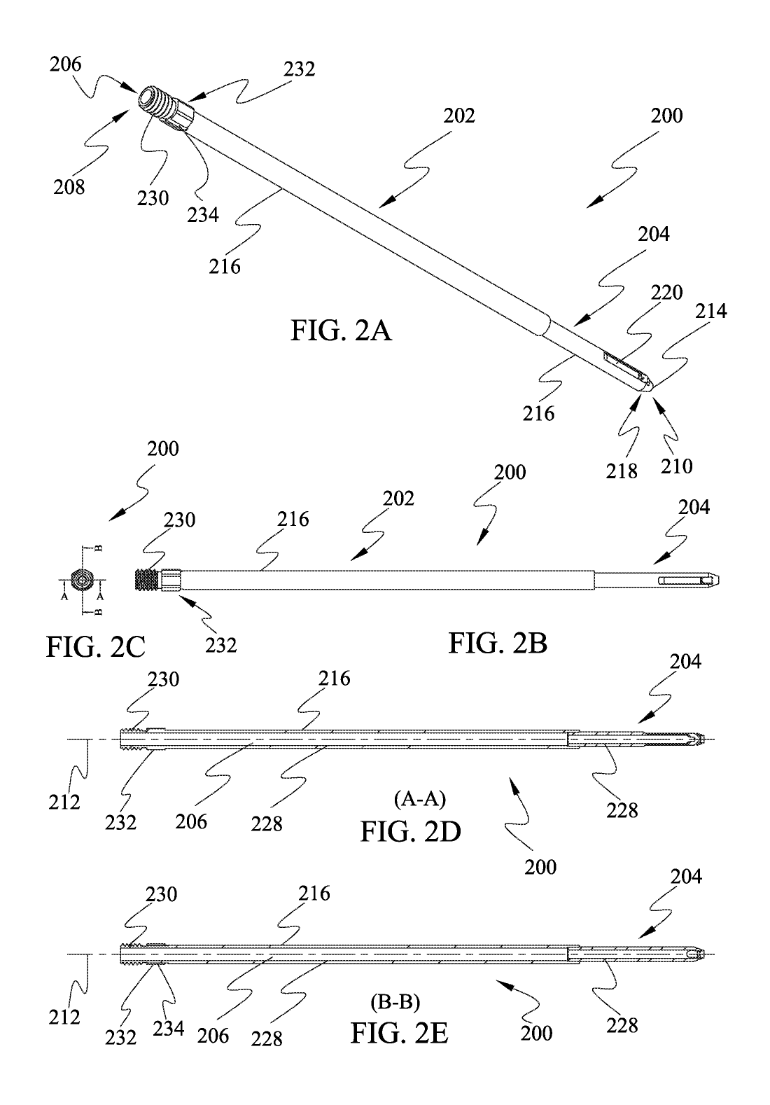

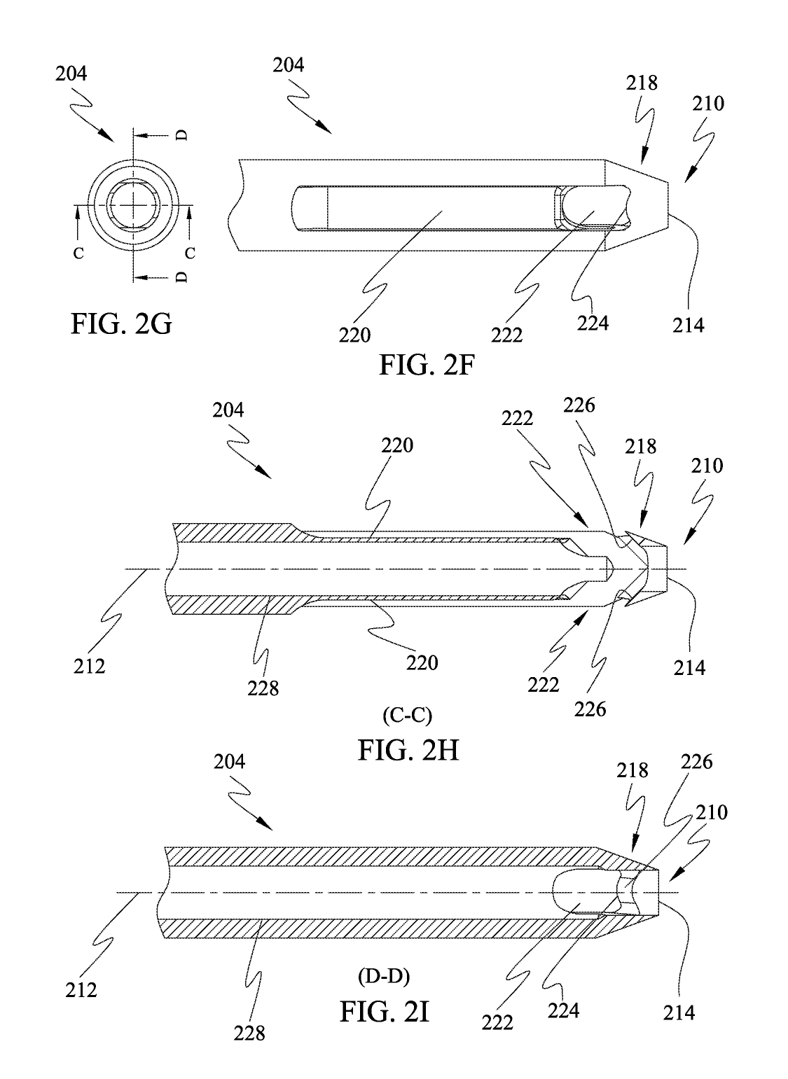

Referring to FIGS. 2A-21, the system 110 may include a first member 200. The first member may be a first means for cutting or coring a tissue. The first member 200 may be a coring cannula. The first member 200 may be configured to be moved below an external surface of the skin. The first member 200 may be a cylindrical member formed of two sub parts. The first part may be a coring cannula base 202 and the second part may be a coring cannula 204. The first member 200 may instead be a monolithic part. The coring cannula base 202 and the coring cannula 204 may be engaged, assembled and/or mated to form the first member 200. The first member 200 may define a first bore 206 extending from a distal end 210 to a proximal end 208 of the first member 200 along the longitudinal axis 212 of the first member 200.

A cutting edge 214 may be defined at the distal end 210. The cutting edge 214 may be configured to cut through the tissue. A portion of the external cylindrical surface 216 of the first member 200 towards the distal end 210 may be inclined or may converge towards the longitudinal axis 212 until it reaches the cutting edge 214, to define a conical configuration 218.

The first member 200 may include at least one slot (any number of slots may be provided) and at least one aperture. Alternatively, the first member 200 may include a pair of slots 220 and a pair of apertures 222. The slots 220 may be recessed into the external surface 216 of the first member 200 close to the distal end 210 as compared to the proximal end 208. The slots 220 may be disposed diametrically opposite to each other, or may be placed in any variety of configurations.

Each of the apertures 222 may be defined between the cutting edge 214 and the slots 220. The apertures 222 may be defined diametrically opposite to each other. Each of the apertures 222 may adjoin corresponding slots 220. The aperture 222 may be defined such that an arched edge 224 bulging towards the slot 220 may be defined on the external surface 216. The edge 224 may be arched, non-arched, or may include any other structure or surface profile in order to form the edge 224. The aperture 222 may be defined by an inclined surface 226 disposed between the arched edge 224 and an inside surface 228 of the first member 200.

A threaded surface 230 may be defined at the proximal end 208 of the first member 200. A shoulder 232 may be defined adjacent to the threaded surface 230. The shoulder 232 may define one or more slots 234.

Provision of the coring cannula 204 that may be adapted with the coring cannula base 202 may enable replacement of the coring cannula 204 with an alternate coring cannula which may have a desired cutting diameter.

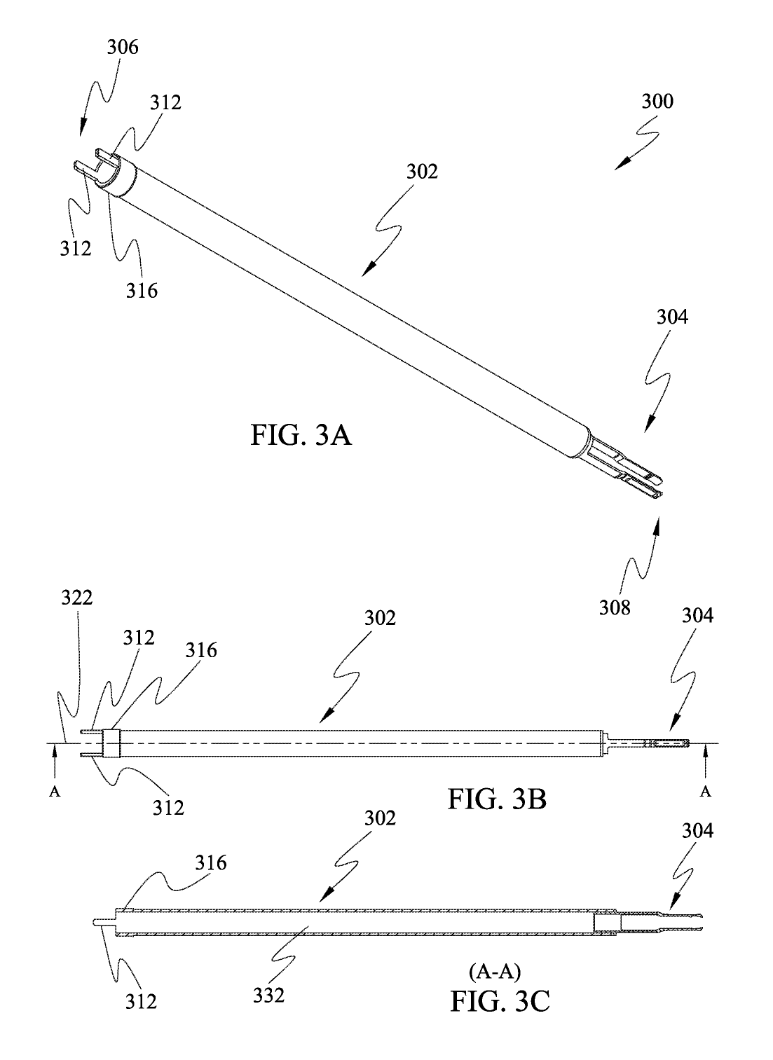

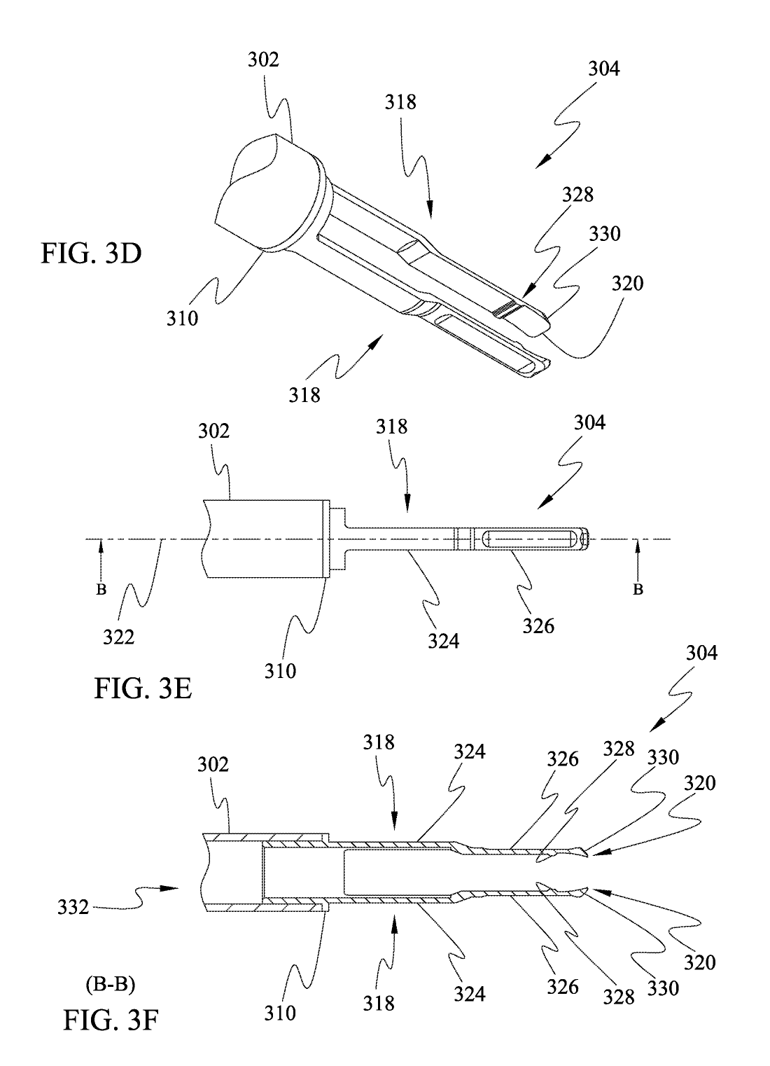

Referring to FIGS. 3A-3F, the system 110 may include a second member 300. The second member 300 may be a second means for clipping the tissue. The second member 300 may be formed of two components, namely a clipping cannula base 302 and a clipping cannula 304. Alternatively, the second member 300 may be a monolithic part. The second member 300 may have a proximal end 306 and a distal end 308. The clipping cannula 304 and the clipping cannula base 302 may be engaged, for example by friction fit, welding, or other connection, to form the second member 300, such that the clipping cannula 304 is disposed towards the distal end 308. The clipping cannula 304 may define an annular ring 310 over its external surface. The annular ring 310 may interface with the edge of the clipping cannula base 302, thereby restricting the clipping cannula base 302 from sliding further towards the distal end 308 of the second member 300.

At the proximal end 306 of the second member 300 one or more longitudinally extending engagement protrusions 312 may be provided. The engagement protrusions 312 may be received by the slots 234 defined in the shoulder 232 provided in the first member 200, so that the torque from the first member 200 is transferred to the second member 300. The engagement protrusions 312 and slots 234 configuration may enable alignment of the arms 318 with the slots 220. An annular protrusion 316 may be provided adjacent to the engagement protrusions.

At the distal end 308 of the second member 300, one or more arms 318 may be provided. In the figures a pair of arms 318 is illustrated. The pair of arms 318 may define a tip 320 at the distal end 308. The pair of arms 318 may be disposed diametrically opposite to each other. Each arm 318 may have a first portion 324 that may be parallel to the longitudinal axis 322 of the second member 300. Further, the arm 318 may include a second portion 326, which may also be parallel to the longitudinal axis 322. However, the second portion 326 may be closer to the longitudinal axis 322, as compared to the first portion 324. The arm 318 may include an intermediate portion, which may be inclined, and may be present between the first portion 324 and the second portion 326. An inner surface of the second portion 326 may define one or more protrusions 328 extending towards the longitudinal axis 322. The protrusions 328 may reduce friction while sliding the second member 300 or the arm 318 over the slot 222 provided in the first member 200. The protrusions 328 may facilitate bending of the arms 318 towards the longitudinal axis 322, when the second member 300 is slid over the first member 200.

The tip 320 may define a slanted surface 330. The slanted surface 330 may compliment the slanted or inclined surface 226 defining the aperture 222 in the first member 200. The slanted surface 330 of the tip 320 may interface and slide against the inclined surface 226 of the first member 200 when the second member 300 is slid. The interface between the slanted surfaces 330, 226 may enable the arms to translate in to the first bore 206 towards the longitudinal axis 212.

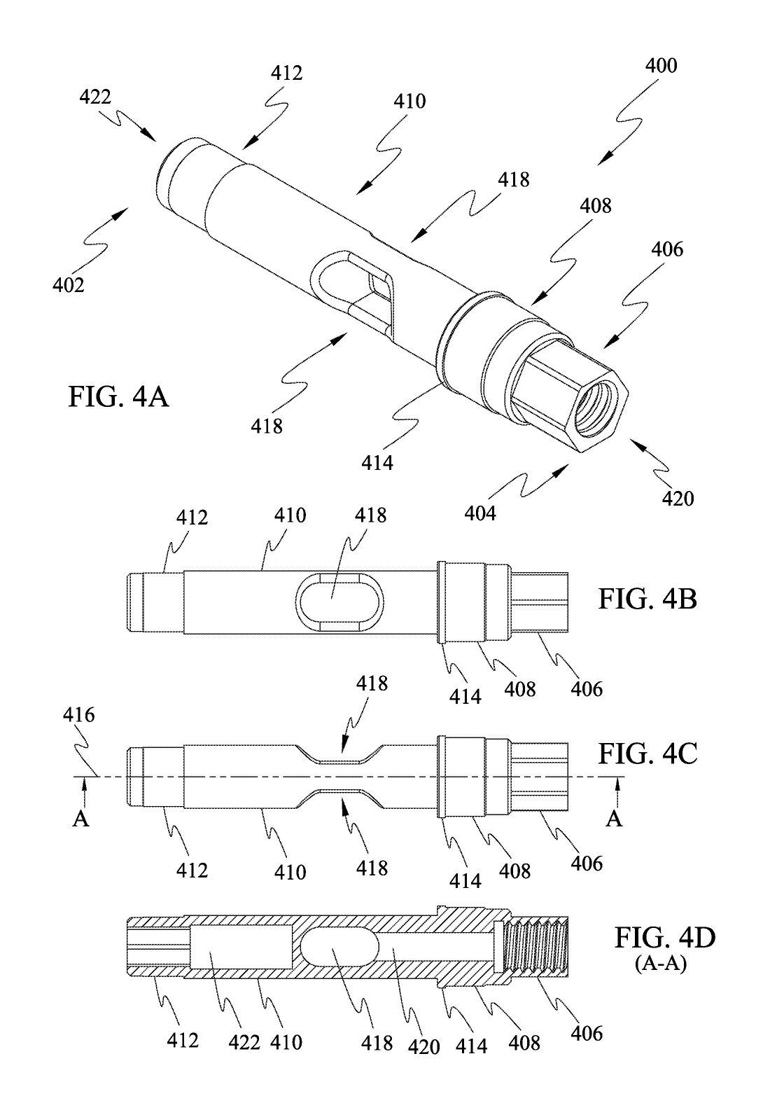

Referring to FIGS. 4A-4D, the system 110 may include an adapter 400. The adapter 400 may have a proximal end 402 and a distal end 404. The adapter 400 may include a first portion 406, a second portion 408, a third portion 410 and a fourth portion 412. The first portion 406 may be provided towards the distal end 404, and may define a hexagonal cross-section. The second portion 408 may adjoin the first portion 406, and may define a circular cross-section. An annular shoulder 414 may be provided in the second portion 408 towards the third portion 410, and may have a diameter greater than the diameter of rest of the second portion 408. The third portion 410 may be adjacent to the annular shoulder 414, and may define a circular cross-section. The fourth portion 412 may be adjacent to the third portion 410, and may define a circular cross-section.

The radial distance of external surface of each of the first portion 406, the second portion 408, a third portion 410 and a fourth portion 412 from a longitudinal axis 416 of the adapter 400 may be different from each of the portions, such that a step is formed between two adjacent external surfaces.

The third portion 410 may define one or more apertures 418. A pair of apertures 418 is illustrated in the figures. The apertures 418 may be defined diametrically opposite to each other. The aperture 418 may be in the form of an opening provided through the external surface of the adapter 400, such that the aperture 418 defines an oblong or oval configuration from a top view. The opening may define a “U” shaped configuration from a side view, such that the arms of the “U” shaped configuration diverge as they extend away from the horizontal portion of the “U” shaped configuration. The edges of the aperture 418 may be curved, without defining hard angles, thereby preventing damage to hair follicles that may pass through the aperture 418.

The adapter 400 may define a bore 420 extending from the distal end 404 until the aperture 418. The bore 420 may have a threaded surface at the distal end to facilitate engagement with the threads 230 of the first member 200.

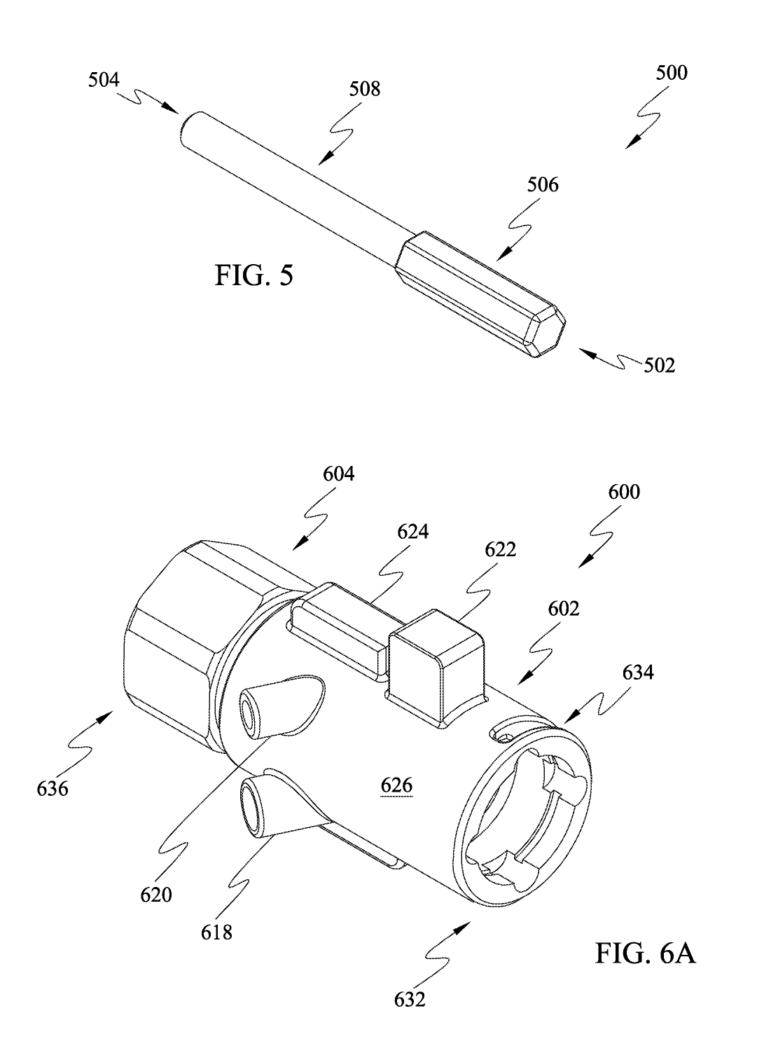

The adapter 400 may further define another bore 422 extending from the proximal end 402 towards the aperture 418, however, the bore 422 may terminate before reaching aperture 418, such that, the bore 422 has only one opening at the proximal end 402. A portion of the inner surface defining the bore 422 may define a polygonal cross section. A power shaft 500 (illustrated in FIG. 5) may be received in the bore 422 such that the power shaft is capable of translating in the bore 422, while transferring torque to the adapter 400, owing to the polygonal cross section of the portion of the hole 422.

Referring to FIG. 5, the system 110 may include a power shaft 500. The power shaft 500 may have a distal end 502 and a proximal end 504. A first portion 506 may be provided towards the distal end 502. A second portion 508 may be defined, such that it starts from one end of the first portion 506 and extends until the proximal end 504.

The first portion 506 may define a hexagonal cross-section or polygonal cross section. The first portion may be received by the bore 422 defined in the adapter 400. The polygonal cross section of the first portion 506 may enable transferring of the torque to the adapter 400, when the power shaft 500 is rotated. The second portion 508 may define a circular cross-section, and may be engaged to a power tool that is capable of rotating the power shaft 500. The power shaft 500 may be a part of a power tool.

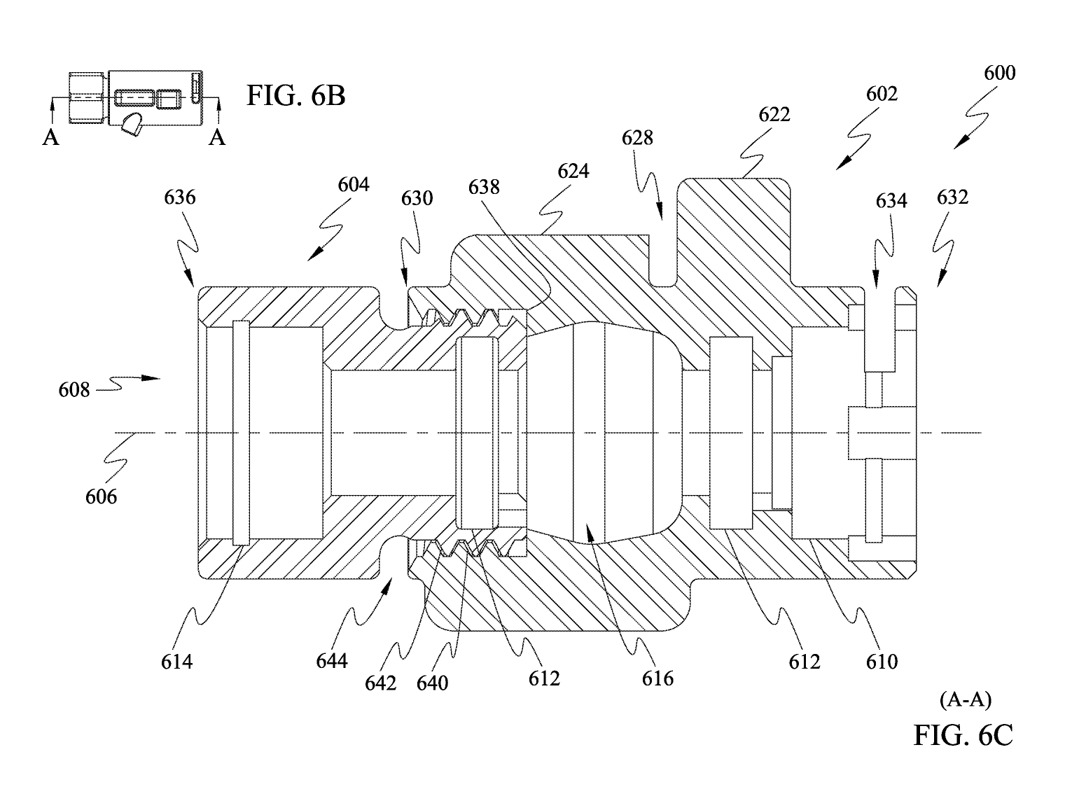

Referring to FIGS. 6A-6C, the system 110 may include a carriage 600. The carriage 600 may include two sub parts, namely a first part 602 and a second part 604. Alternatively, the carriage may be made as a single unit. The carriage 600 may have a longitudinal axis 606, and a bore 608 may be defined along the longitudinal axis 606. The bore 608 may define a first bearing engagement surface 610, a plurality of o-ring engagement surfaces 612, a retention ring engagement surface 614. The bore 608 may define a chamber 616 between the plurality of o-ring engagement surfaces 612, such that, the third portion 410 having the aperture 418 of the adapter 400 may be encompassed in the chamber 616. An outlet port 618 and a flush port 620 may be defined such that the outlet port 618 and the flush port 620 are in fluidic communication with the chamber 616. Each of the outlet port 618 and the flush port 620 may include a protrusion extending from an outer surface 626 of the carriage 600.

A first pillar 622 and a second pillar 624 may extend laterally from a cylindrical outer surface 626 of the carriage 600. The first pillar 622 and the second pillar 624 may be configured such that a gap 628 may be defined between the first pillar 622 and the second pillar 624. The first part 602 may have a proximal end 630 and a distal end 632. Near the distal end 632 of the first part 602, a slot 634 may be provided, which may extend from the outer surface 626 towards the bore 608.

The first part 602 may have an internally threaded surface 640 at the proximal end 630. The threaded configuration 640 may terminate before extending into the chamber 616. The second part 604 may include an external threaded surface 642 towards the distal end 638. The external surface of rest of the second part 604 may define a polygonal cross section.

When the first part 602 and the second part 604 are assembled, an annular groove 644 may be defined, which may receive an O-ring.

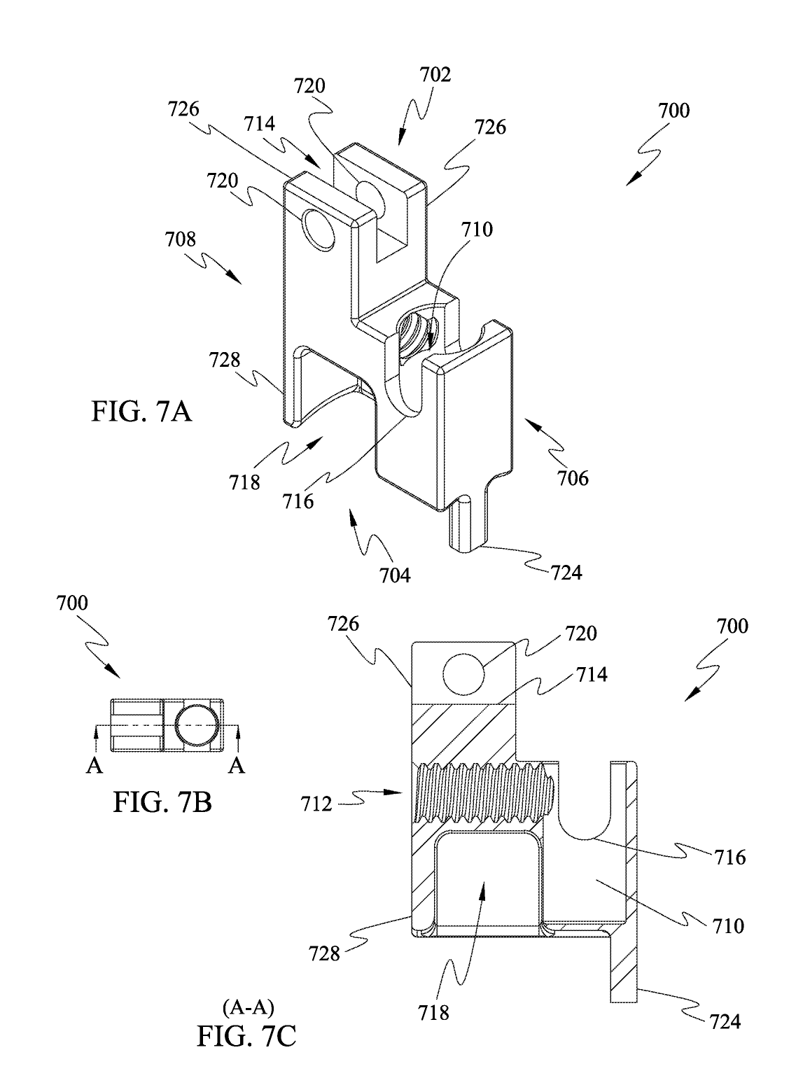

Referring to FIGS. 7A-7C, the system 110 may include a first connecting member 700. The first connecting member 700 may have a superior end 702, an inferior end 704, a distal end 706 and a proximal end 708.

The first connecting member 700 may include a cavity 710, a threaded hole 712, a first slot 714, a pair of second slots 716, a third slot 718 and a pair of pin holes 720.

The cavity 710 may be defined in the superior-inferior direction. The pair of second slots 716 may be through slots that are open towards the superior side, and may be disposed opposite to each other. The surface of the first connecting member 700 that may define the cavity, may also define the pair of second slots 716. A post 724 may extend from a portion of the first connecting member 700 that defines the cavity, in the inferior direction. The third slot 718 may be defined between the portion of the first connecting member 700 that defines the cavity 710 and a protrusion 728 extending in the inferior direction. The third slot 718 may be open on the inferior side and on one of the lateral sides. A portion of the first connecting member 700 disposed over the third slot 718 may define a threaded bore 712. The threaded bore 712 may extend from the proximal end 708 towards the distal end 706 such that an end of the bore 712 is exposed to the cavity 710. A portion of the first connecting member 700 disposed over the threaded bore 712 may include a pair of arms 726. The pair of arms 726 may be parallel to each other and may be spaced apart to define the first slot 714. The pair of arms 726 may extend in the superior direction. Each of the arms 726 may define a pin hole 720, such that the pin holes 720 defined in the arms 726 may oppose each other. The first connecting member 700 may form a part of a locking assembly.

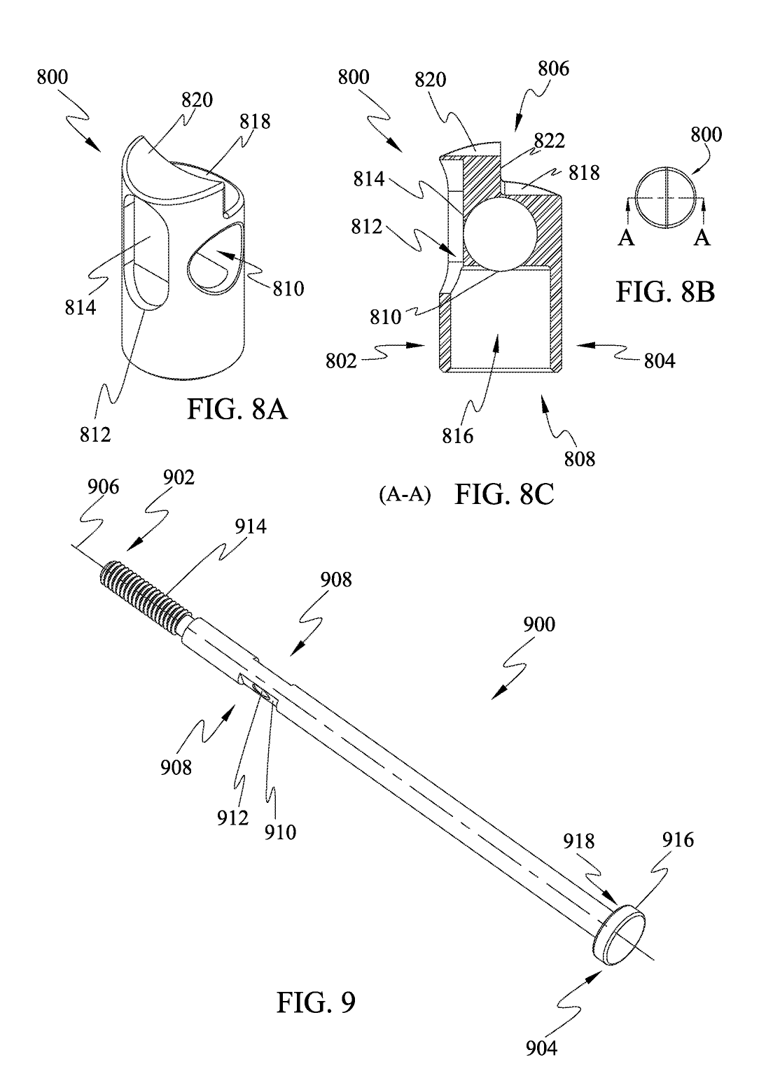

Referring to FIGS. 8A-8C, the system 110 may include a stop 800, which may be received by the cavity 710 defined in the first connecting member 700. The stop 800 may have a proximal end 802, a distal end 804, a superior end 806 and an inferior end 808. A through hole 810 may be defined laterally extending across a longitudinal axis of the stop 800. A first slot 812 may be defined by a recessed surface disposed towards the proximal end 802. The first slot 812 may terminate before it reaches the hole 810, such that, a first supporting wall 814 may be formed. A cavity 816 may be defined from the inferior end 808 towards the superior end 806, and may terminate before reaching the through hole 810. The longitudinal axis of the cavity 816 may be perpendicular to the longitudinal axis of the through hole 810.

A portion of the stop 800 towards its superior side may include a first seat 818, a second seat 820 and a block wall 822. The first seat 818 and the second seat 820 may define a concave configuration, which may be configured to interface with a component defining a complimentary configuration. The second seat 820 and the first seat 818 may be generally horizontal, while the block wall 822 may be vertically disposed. The second seat 820, the block wall 822 and the first seat 818 may define a cascade configuration. The stop 800 may form a part of the locking assembly.

Referring to FIG. 9, the system 110 may include a first arm 900 that may be connected to the first member 200 through the first connecting member 700. The first arm 900 may have a proximal end 902, a distal end 904 and a longitudinal axis 906. The first arm 900 may be cylindrical shaft. A pair of slot 908 may be defined on either sides of the longitudinal axis 906, which may define pair of adapting surfaces 910 on either sides of the longitudinal axis 906. A through hole 912 may be defined, having a longitudinal axis which may be perpendicular to the longitudinal axis 906. The through hole 912 may extend between the slots 908. The first arm 900 may have a threaded portion 914 towards its proximal end 902. The region near the distal end 904 of the first arm 900 may define a cylindrical shoulder 916. The cylindrical shoulder 916 may define an engaging surface 918. The engaging surface 918 may face the proximal end 902.

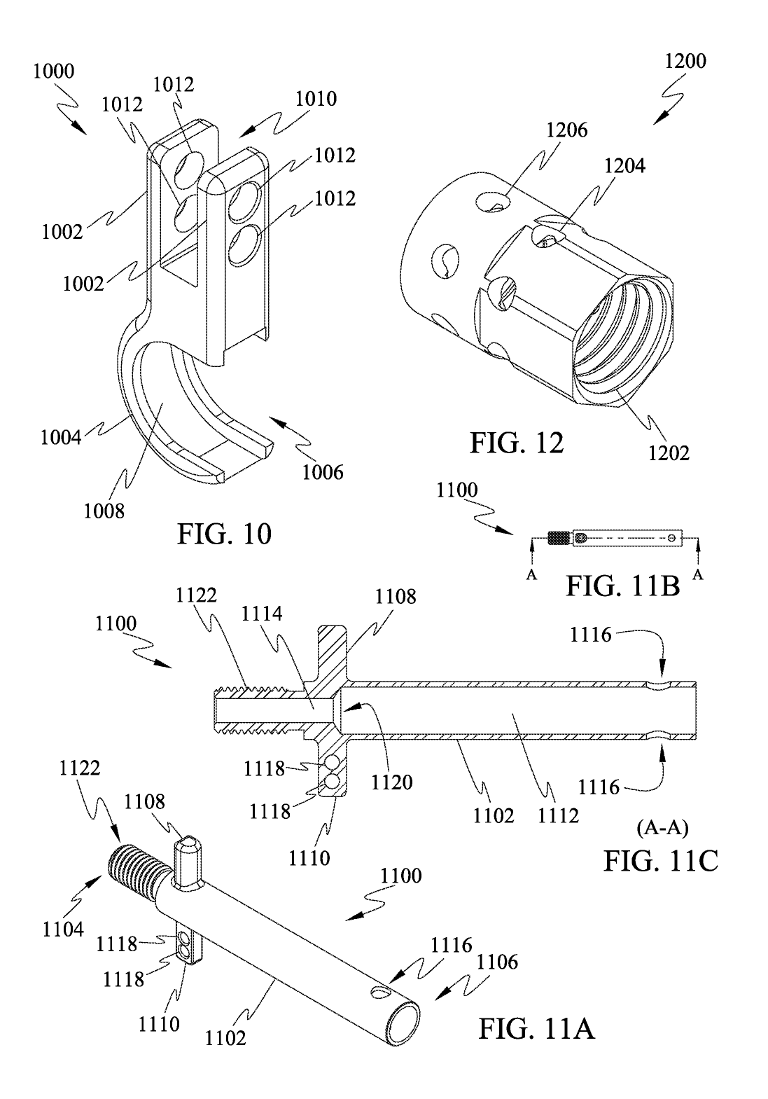

Referring to FIG. 10, the system 110 may include a second connecting member 1000 configured to connect the second member 300 with the second arm 1100. The second connecting member 1000 may include one or more handles 1002 and an arm 1004.

The arm 1004 may define a “C” shaped configuration. The arm 1004 may have an inner surface 1006 which may be recessed to define a groove 1008. The groove 1008 may be configured to receive a bearing.

The handle 1002 may be disposed towards the superior side of the second connecting member 1000. A through slot 1010 extending through opposing surfaces of the handle 1002 may be defined in the handle 1002. A plurality of holes 1012 may be defined in the handle 1002, such that the holes 1012 are exposed to the slot 1010.

Referring to FIGS. 11A-11C, the system 110 may include a second arm 1100 configured to be connected to the second member 300 using the second connecting member 1000. The second arm 1100 may be a cylindrical shaft having an external surface 1102, a proximal end 1104, a distal end 1106, a first post 1108, a second post 1110, a first bore 1112, a second bore 1114, a first aperture 1116 and a pair of second apertures 1118. The first post 1108 and the second post 1110 may extend laterally from the external surface 1102, in opposite directions. The first bore 1112 may extend from the distal end 1106 towards the proximal end 1104 along the longitudinal axis of the second arm 1100. The second bore 1114 may extend from the proximal end 1104 towards the distal end 1106 along the longitudinal axis of the second arm 1100 until it meets the first bore 1112. The second bore 1114 may have a diameter that may be smaller than the diameter of the first bore 1112. The difference in diameter of the first bore 1112 and the second 1114 may create a step 1120. The first aperture 1116 may be a through hole extending through the cylindrical external surface 1102 of the second arm 1100. The first aperture 1116 may be closer to the distal end 1106 as compared to the proximal end 1104.

The first post 1108 and the second post 1110 may be closer to the proximal end 1104 as compared to the distal end 1106. The first post 1108 may extend laterally in the superior direction, while the second post 1110 may extend laterally in the inferior direction. One or more second apertures 1118 may be defined in the second post 1110 to facilitate engagement with the second connecting member 1000.

At least a part of the external surface 1102 between the proximal end 1104 and the posts 1108, 1110 may be threaded 1122.

Referring to FIG. 12, an adjustment sleeve 1200 may be provided for engagement with the second arm 1100. The adjustment sleeve 1200 may be internally threaded 1202. The adjustment sleeve 1200 may be engaged with the second arm 1100 having threads. The adjustment sleeve 1200 may include a first set of apertures 1206 extending from the external surface of the adjustment sleeve 1200 towards the internal surface of the adjustment sleeve 1200. The apertures 1206 may be defined around the diameter of the adjustment sleeve 1200. The adjustment sleeve 1200 may include a second set of apertures 1204 extending from the external surface of the adjustment sleeve 1200 towards the internal surface of the adjustment sleeve 1200. The apertures 1204 may be defined around the diameter of the adjustment sleeve 1200. The first set of apertures 1206 may be offset along the longitudinal axis of the adjustment sleeve 1200, with respect to the second set of apertures 1204. The apertures 1204, 1206 may enable operating the adjustment sleeve 1200 to adjust the placement of the adjustment sleeve 1200 relative to the a second arm 1100. The adjustment sleeve 1200 may form a part of the locking assembly.

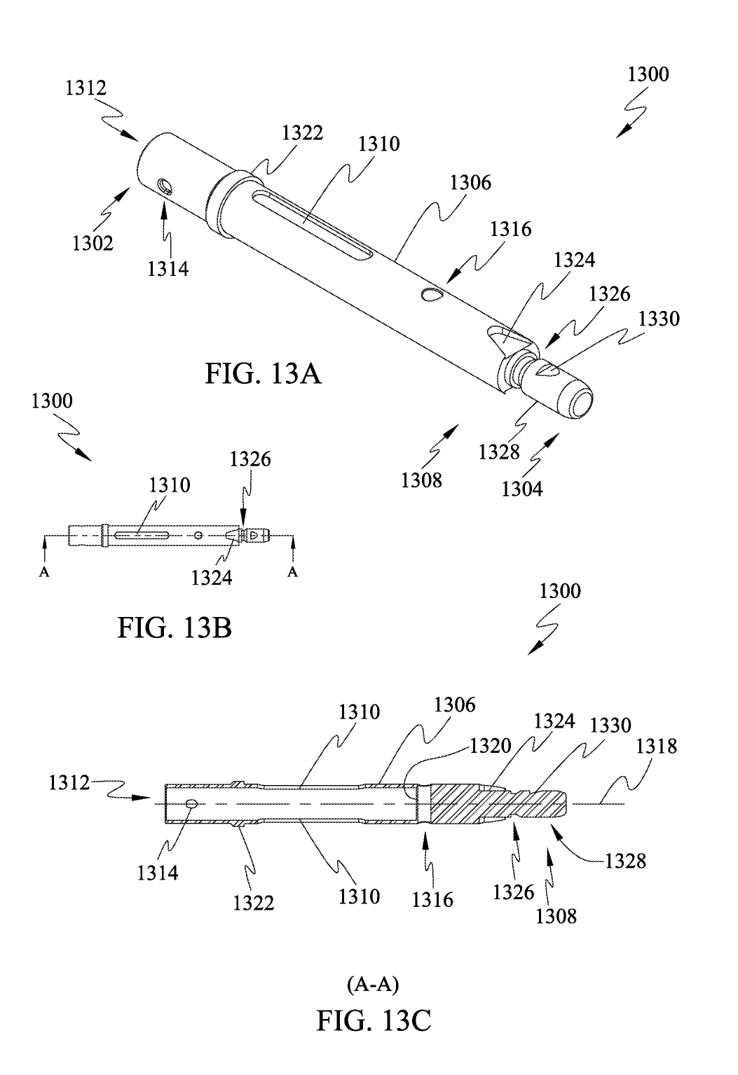

Referring to FIGS. 13A-13C, a stationary arm 1300 may be configured to receive the second arm 1100. The stationary arm 1300 may be a cylindrical member having a proximal end 1302, a distal end 1304, an external surface 1306, an engagement feature 1308, a pair of slots 1310, a first bore 1312, a pair of opposing through holes 1314 and a second bore 1316. The first bore 1312 may extend from the proximal end 1302 towards the distal end 1304, along a longitudinal axis 1318 of the stationary arm 1300. The first bore 1312 may have an opening at the proximal end 1302 and may terminate before reaching the distal end 1304. The termination of the first aperture 1312 before reaching the distal end 1304 may result in the formation of a supporting wall 1320. The internal diameter of the first bore 1312 may be larger than the external diameter of the second arm 1100, thereby enabling the second arm 1100 to be received within the first bore 1312, and allowing the second arm 1100 to translate along the first bore 1312. The pair of slots 1310 may be defined to oppose each other, and may extend from the external surface 1306 into the first bore 1312. A flange 1322 may be defined on the external surface 1306, and may be provided between the slots 1310 and the proximal end 1302. The pair of opposing through holes 1314 may extend from the external surface 1306 into the first bore 1312. The pair of holes 1314 may be defined between the flange 1322 and the proximal end 1302. The second bore 1316 may be defined between the slots 1310 and the distal end 1304. The engagement feature 1308 may be provided towards the distal end 1304. The engagement feature 1308 may include a pair of recessed portions 1324, a neck portion 1326, a head portion 1328 and a second recessed portion 1330. The head portion 1328 may be a cylindrical shaped body provided towards the distal end 1304. The neck portion 1326 may be provided between a portion of the arm 1300 that defines the recess 1324 and the head portion 1328. The neck portion 1326 may have a diameter that is smaller than the diameter of the head portion 1328. The pair of recessed portions 1324 may be disposed on opposite sides of the external surface 1306. The recess 1324 may define a “V” or “U” shaped configuration. The second recessed portion 1330 may be defined on the cylindrical surface of the head portion 1328. The second recessed portion 1330 may be defined a “D” shaped configuration. The engagement feature 1308 may enable the stationary arm 1300 to be engaged with a component or system that may include a pair of paddles.

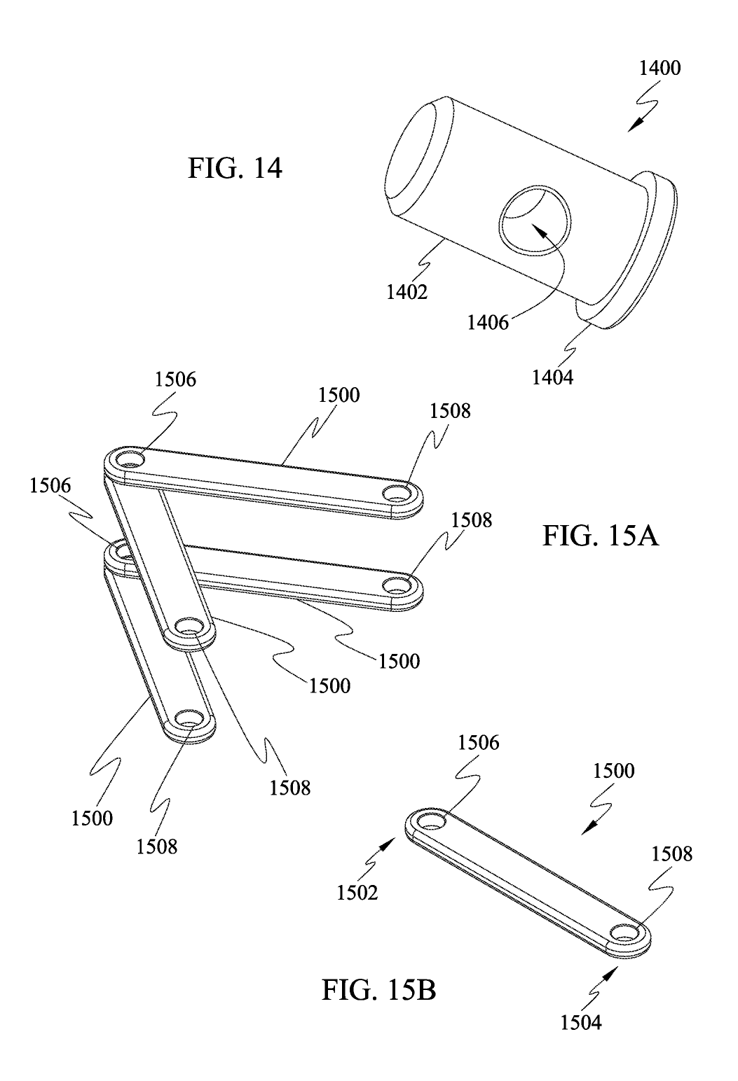

Referring to FIG. 14, the system 110 may include a second arm cap 1400. The second arm cap 1400 may be engaged with the second arm 1100. The second arm cap 1400 may include a shaft 1402 and a head 1404. A through hole 1406 may be defined in the shaft 1402. The through hole 1406 may be defined perpendicularly to the longitudinal axis of the shaft 1402. The head 1404 may have a diameter that is larger than the diameter of the first bore 1112 defined in the second arm 1100 at its distal end 1106. The shaft 1402 may be received into the second arm 1100 through an opening at it distal end 1106. The head 1404 may rest against or disposed outside the opening at the distal end 1106 of the second arm 1100.

Referring to FIG. 15, the system 110 may include two pairs of paddle link members 1500. The paddle link member 1500 may have a proximal end 1502 and a distal end 1504. The link member 1500 may define two through holes. A first through hole 1506 may be defined closer to the proximal end 1502, and may extend through two opposing surfaces of the link member 1500. A second through hole 1508 may be defined closer to the distal end 1504, and may extend through the two opposing surfaces of the link member 1500.

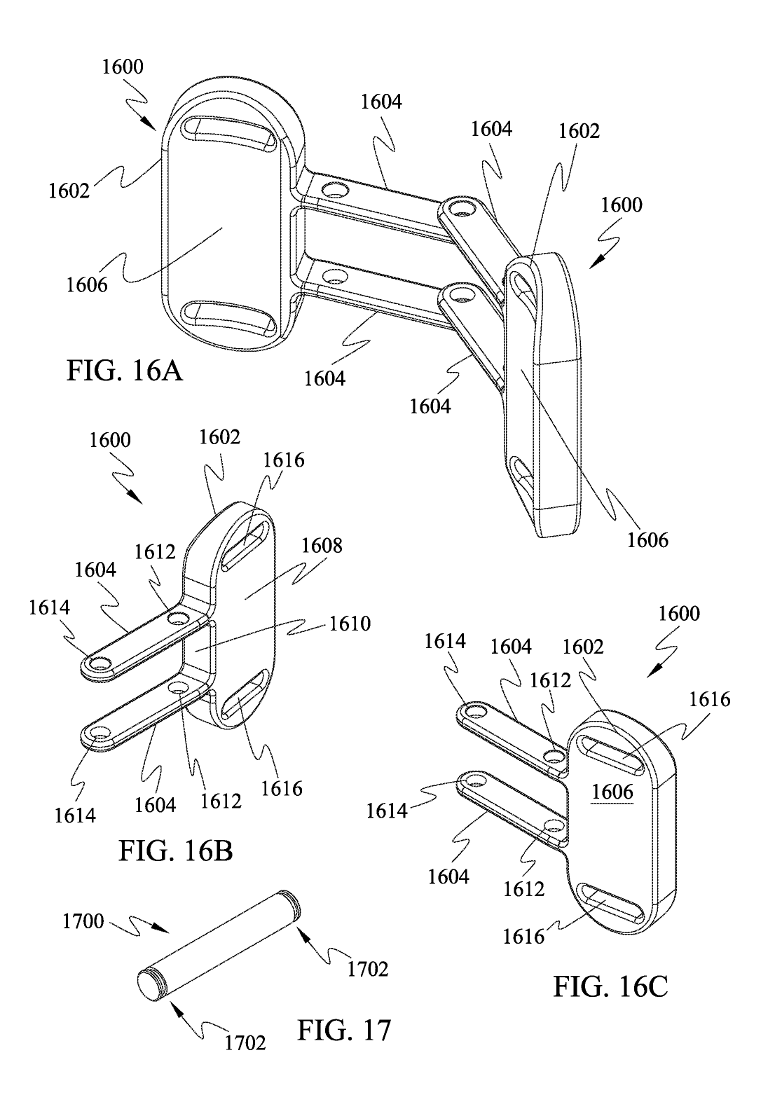

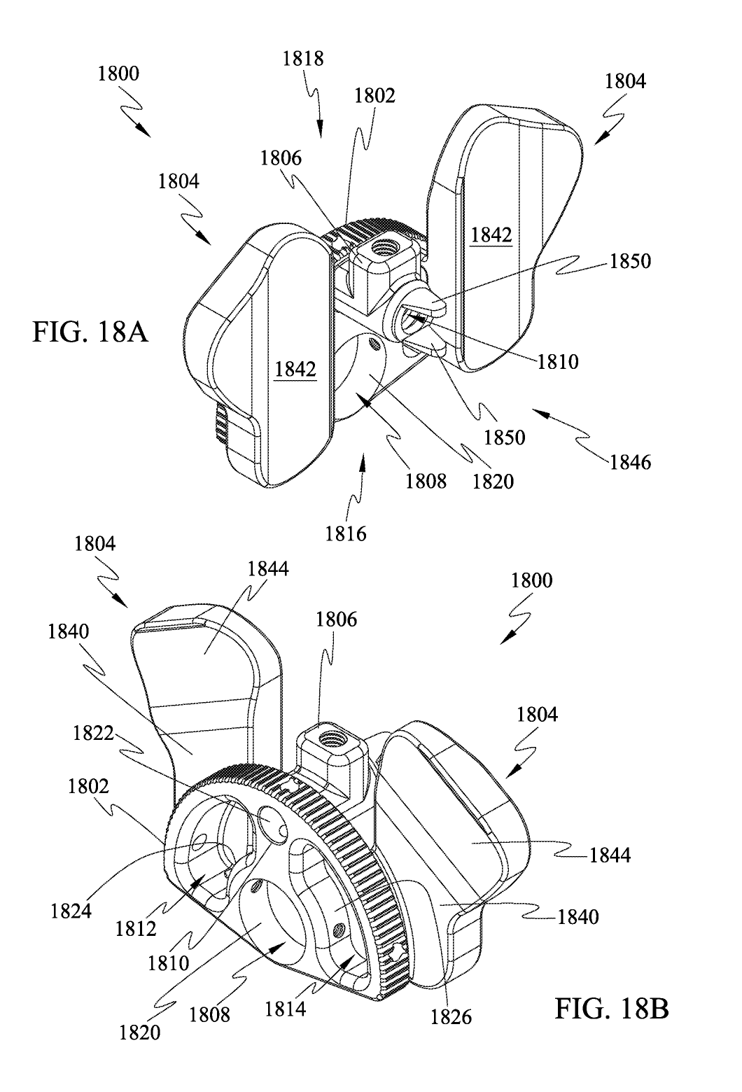

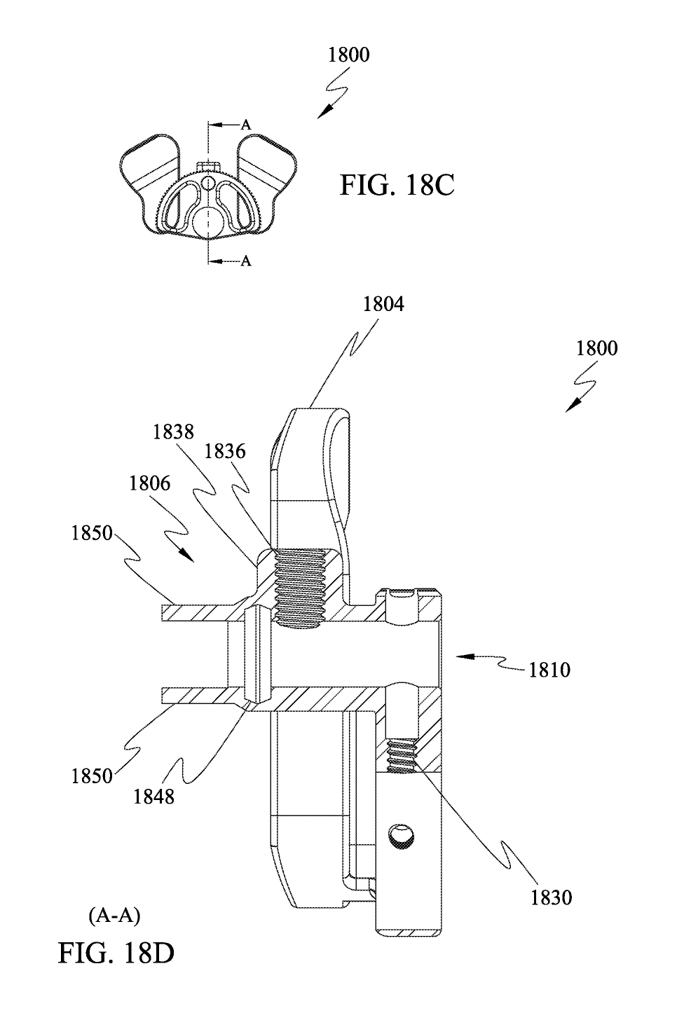

Referring to FIGS. 16A-16C, the system 110 may include a pair of first paddle members 1600. The first paddle member 1600 may include a first paddle 1602 and a pair of link members 1604. The first paddle 1602 may be an oblong shaped body having a first surface 1606 and an opposing second surface 1608. A third surface 1610 may extend between the first surface 1606 and the second surface 1608. The first paddle 1602 may be designed to define a shape or topography that may facilitate a user to hold on to the paddles 1602, for example using four fingers (leaving the thumb) of each of his hands, and pull the paddles 1602 towards a pair of second paddles 1804 (illustrated in FIG. 18A-18D). The first surface 1606 may define a concave curvature that may facilitate a user to hold on to the paddles 1602 and apply the desired force. The link members 1604 may extend from the third surface 1610 laterally on the same side of the third surface 1610. The paddle link members 1604 may be spaced apart. Each pair of link members 1604 may define two through holes. A first through hole 1612 may be defined close to the intersection between the link member 1604 and the third surface 1610, and may extend through two opposing surfaces of the link member 1604. A second through hole 1614 may be defined close to the free end of the link member 1604, and may extend through the two opposing surfaces of the link member 1604. Further one or more slots 1616 may be defined on the first surface 1606. The slots 1616 may be configured to facilitate better gripping of the first paddle 1602 by a user. The slots 1616 may receive one or more loops, straps, Velcro, levers, or other structure that may enable advancing and/or retracting (or even omnidirectional control, handling, or manipulation) of the paddles 1602. The slots 1616 may be substantially perpendicular to a longitudinal axis of the first paddle 1602. The slots 1616 may be in line with the direction in which the fingers are held while engaging the paddles 1602. The slots 1616 may extend through the first surface 1606 and the second surface 1608.

Referring to FIG. 17, the system 110 may include multiple link member pins 1700. The link member pin 1700 may be received by through holes 1612, 1614 defined in the paddle link members. Each link member pin 1700 may define an annular groove 1702 close to each of the ends of the link member pin 1700. Each of the grooves 1702 may be configured to receive a retaining ring, thereby ensuring that the pin 1700 is not disengaged from the link members 1500.

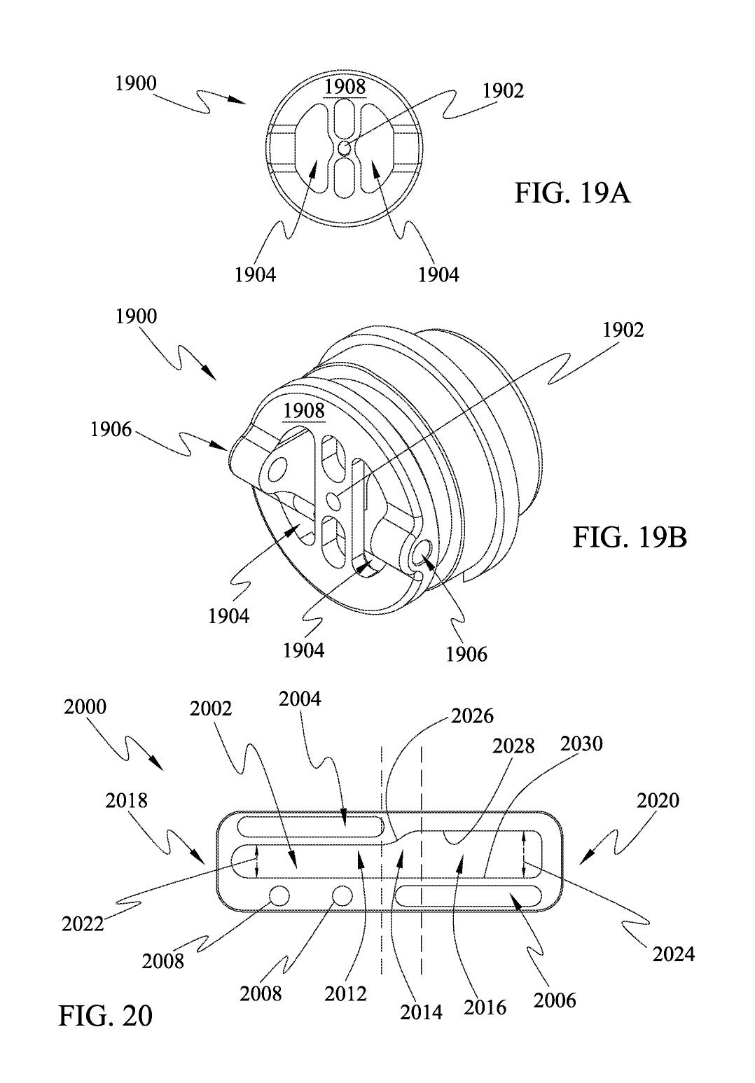

Referring to FIG. 18A-18D, the system 110 may include an external supporting member 1800. The external supporting member 1800 may include an arch portion 1802, a pair of second paddles 1804, an elongated housing 1806. The arch 1802 may define a first aperture 1808, a second aperture 1810, a third aperture 1812 and fourth aperture 1814. The first aperture 1808 may be configured to accommodate a counter pressure device 1900 (illustrated in FIGS. 19A-19B). The first aperture 1808 may be provided towards the inferior end 1816 of the external supporting member 1800. The second aperture 1810 may be provided above the first aperture 1808. The second aperture 1810 may be provided towards the superior end 1818 of the external supporting member 1800. The third aperture 1812 and the fourth 1814 may be provided adjacent to the first aperture 1808, and on either sides of the first aperture 1808. As a result of providing the first aperture 1808, the second aperture 1810, the third aperture 1812 and the fourth aperture 1814, a first inner surface 1820, a second inner surface 1822, a third inner surface 1824 and a fourth inner surface 1826, respectively, may be defined. A first threaded hole 1830 may be provided, extending from the first inner surface 1820 to the second inner surface 1822. A second threaded hole may extend from the first inner surface 1820 to the third inner surface 1824. A third threaded hole may be provided extending from the first inner surface 1820 to the fourth inner surface 1826.

A pair of second paddles 1804 may be connected to the arch 1802. Each of the second paddles 1804 may be disposed on either sides of the vertical axis of the arch 1802. The external supporting member 1800 may include an elongated housing 1806. The elongated housing 1806 may be provided between the two paddles 1804.

The second paddle 1804 may be a P-shaped body having a first surface 1840 and an opposing second surface 1842, which may face the first pair of paddle members 1600. The second paddles 1804 may be disposed such that they form a mirror image of each other. The paddle 1804 may be designed to define a shape or topography that may facilitate a user to hold on to the paddles 1804, for example using thumb of each of his hands, and pulling the paddles 1602 of first paddle members 1600 towards the second paddles 1804. The first surface 1840 may define a concave curvature 1844 towards the superior end 1818 of the paddle 10. The longitudinal axis of the concave curvature 1844 may be oblique to the longitudinal axis of the paddle 1804. The concave curvature 1844 may facilitate pressing of the user's thumb against the surface that defines the concave curvature 1844. The length of the concave curvature 1844 may be such that a major portion of the thumb may be supported. The concave curvature 1844 on the first surface 1840 may define a surface having a suitable length, curvature and angle of inclination, to compliment the shape and length of the thumb, and the angle at which the user holds and presses the thumb against the paddle 1804.

The second aperture 1810 may extend from the arch 1802 into and through the elongated housing 1806. Near the proximal end 1846 of the second aperture 1810 an annular slot 1848 may be defined. Near the proximal end of the elongated housing 1806 a pair of protrusions 1850 may be provided. The protrusions 1850 may be in the form of “V” or “U” shaped extensions, which may compliment the recessed portions 1324 provided in the stationary member 1300. On top of the elongated housing 1806, a protrusion 1838 may be provided, such that, it may be perpendicular to the longitudinal axis of the second aperture 1810. The protrusion 1838 may further define a threaded hole 1836 which may meet the second aperture 1810 in the elongated housing 1806.

The external supporting member 1800 may include a counter pressure device 1900. The counter pressure device 1900 may be engaged with the external supporting member 1800 such that a surface of the external supporting member 1800 interfaces with and presses against the external surface of the skin, while in use. Referring to FIGS. 19A-19B, the counter pressure device 1900 may be a circular plate with a targeting aperture 1902, which may be defined at the center of the plate. The counter pressure device 1900 may further define piercing member accommodation opening 1904 defined on both sides of the targeting aperture 1902. The piercing members may translate to-and-fro through the piercing member accommodation opening 1904. Further, the piercing member accommodation opening 1904 may define an oblong shape so that the piercing members may pivot while the piercing members have extended through the piercing member accommodation opening 1904 (counter pressure device 1900).

The counter pressure device 1900 may define a pair of pivot holes 1906. The longitudinal axis of the pivot holes 1906 may be perpendicular to the longitudinal axis of the targeting aperture 1902. The pivot holes 1906 may be defined such that a surface 1908 of the counter pressure device 1900 that interfaces with the skin is flush, without protrusions.

The counter pressure device 1900 may apply pressure to, over or around the tissue comprising hair follicle when it is held against the external surface of the skin.

Referring to FIG. 20, the system 110 may include a guide plate 2000. The guide plate 2000 may be rectangular in shape whose corners may be filleted. The guide plate 2000 may include a first slot 2002, a second slot 2004, a third slot 2006 and a plurality of holes 2008.

The first slot 2002, second slot 2004 and third slot 2006 may be through slots. The first slot 2002 may be defined between the second slot 2004 and the third slot 2006. The second slot 2004 may be defined near the proximal superior side of the guide plate 2000, while the third slot may be defined near the distal inferior side of the guide plate 2000. The second slot 2004 and the third slot 2006 may have uniform width and length. The plurality of holes 2008 may be defined opposite to the second slot 2004, and may be on the same side of the third slot 2006.

The first slot 2002 may define a first region 2012, a second region 2014 and a third region 2016. The first region 2012 may be close to a proximal end 2018 of the guide plate 2000. The third region 2016 may be close to a distal end 2020 of the guide plate 2000. The second region 2014 may be intermediate the first region 2012 and the third region 2016. The first region 2012 may have the first width 2022, and the third region 2016 may have a third width 2024. The first width 2022 may be smaller than the third width 2024. The second region 2014 may have a width that is same as the first width 2022 at first end, and a width that is same as the third width 2024 at the opposite second end. The width of the second region 2014 may gradually change from first width 2022 to third width 2024. The gradual change in width in the second region 2014 may define a curved surface 2026. The curved surface 2026 may be defined in the superior edge 2028 of the first slot 2002, whereas the inferior edge 2030 of the slot 2002 may define a straight line.

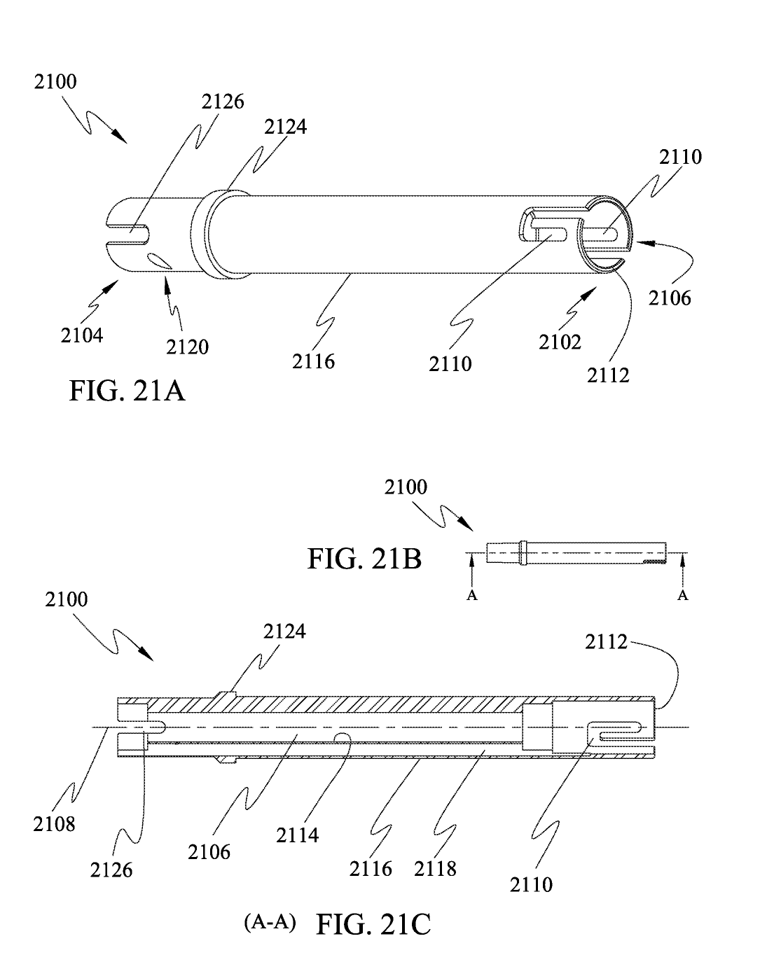

Referring to FIGS. 21A-21C, the system 110 may include a cover member 2100. The cover member 2100 may be configured to receive the first member 200 and the second member 300. The cover member 2100 may be a cylindrical tube having a proximal end 2104 and a distal end 2102. The cover member 2100 may define a bore 2106 along its longitudinal axis 2108, extending from the proximal end 2104 to the distal end 2102. The distal end 2102 of the cover member 2100 may include a U shaped slot 2110 defined on its cylindrical body. The slot 2110 may extend from the edge 2112 at the distal end 2102 of the cover member 2100 along the longitudinal axis 2108 of the cover member 2100 and thereafter turn towards the distal end 2102 of the cover member 2100 and terminate before reaching the edge 2112 at the distal end 2102, thereby defining the “U” shape. The cover member 2100 may have a pair of such slots 2110 defined on diametrically opposing sides of the cylindrical body.

The cover member 2100 may have an inner surface 2114 and an outer surface 2116. A channel 2118 may be defined between the inner surface 2114 and the outer surface 2116 such that fluid may be passed through the channel 2118. The channel 2118 may be defined between the proximal end 2104 and the distal end 2102. The channel 2118 may extend from the proximal end 2104 until the distal end 2102. An aperture 2120 may be defined in the cover member 2100. The aperture 2120 may extend from the outer surface 2116 into the channel 2118. The fluid may be passed into the channel 2118 through the aperture 2120. The aperture 2120 may be defined closer to the proximal end 2104 than the distal end 2102.

The inner surface 2114 of the cover member 2100 may define a stepped configuration, such that bushing may be accommodated in the stepped configuration. The bushing may define a channel that may be in fluidic communication with the channel 2118.

A flange 2124 may be defined on the outer surface 2116 closer to the proximal end 2104. A pair of opposing slots 2126 may be defined by the cover member 2100. Each of the slots 2126 may be defined on diametrically opposing sides of the cover member 2100. Each slot 2126 may start from the edge of the cover member 2100 at the proximal end 2104 and may terminate before reaching the flange 2124.

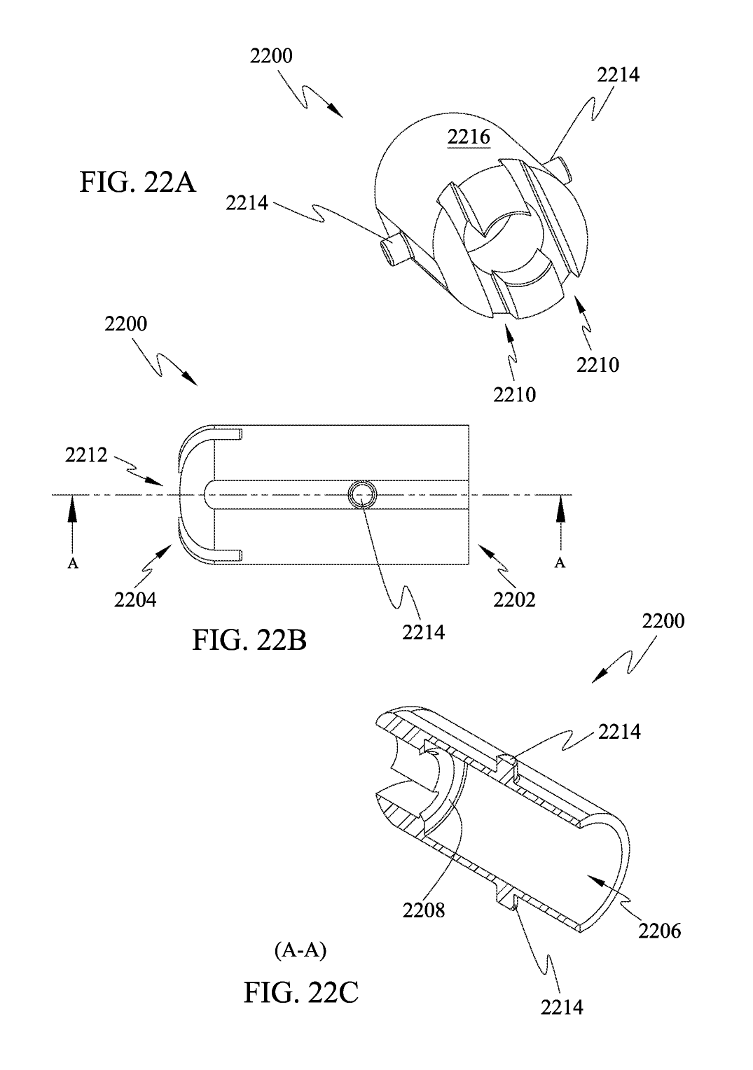

Referring to FIGS. 22A-22C, the system 110 may include a tissue stabilizing member 2200. The tissue stabilizing member 2200 may be moved below the external surface of the skin. The tissue stabilizing member 2200 may translate parallel to the longitudinal axis of the first member 200 or the longitudinal axis of a cover member 2100. The tissue stabilizing member 2200 may apply pressure at least around the tissue disposed between the tissue stabilizing member 2200 and the counter pressure device 1900. The tissue stabilizing member 2200 may be engaged with the cover member 2100.

The tissue stabilizing member 2200 may define a substantially cylindrical shape. The tissue stabilizing member 2200 may have a proximal end 2202 and a distal end 2204. The tissue stabilizing member 2200 may define a bore 2206 extending from the proximal end 2202 until the distal end 2204. A flange 2208 may be defined on an inner surface of the tissue stabilizing member 2200, such that the inside diameter of tissue stabilizing member 2200 at the flange and beyond is smaller than the inside diameter of the tissue stabilizing member 2200 at the proximal end 2202. The bore 2206 defined between the proximal end 2202 and the flange 2208 may be configured to receive a compression member, such as a spring. The compression member may press against the flange 2208 when the tissue stabilizing member is pushed in to the cover member 2100. A pair of slots 2210 may be defined on a face 2212 of the tissue stabilizing member 2200. The pair of slots 2210 may enable piercing members, which may have pierced into a tissue through an external surface of the skin, to pivot, even if the piercing members have pierced beyond the distal end of the tissue stabilizing member 2200. The tissue stabilizing member 2200 may include a pair of pins 2214 laterally extending from an outer surface 2216 of the tissue stabilizing member 2200 in opposing directions. The pins 2214 may be received in the “U” shaped slots 2110 defined in the cover member 2100, thereby enabling the tissue stabilizing member 2200 to translate parallel to the longitudinal axis of the first member 200 or the longitudinal axis of the cover member 2100.

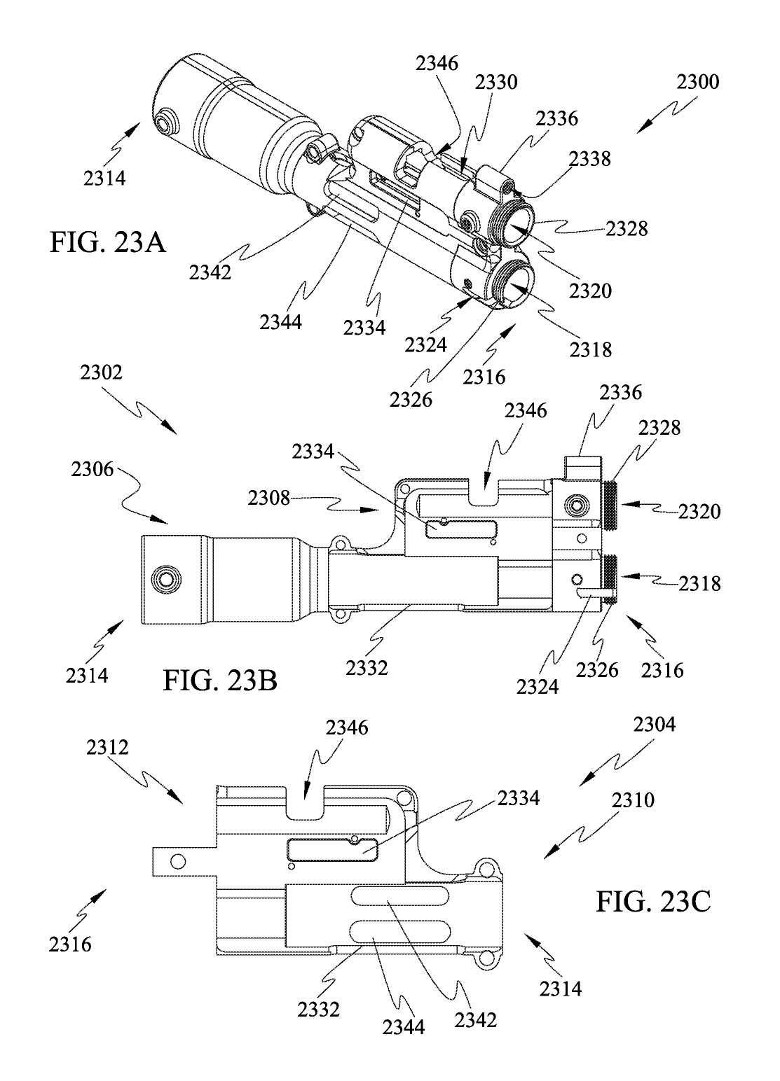

Referring to FIGS. 23A-23C, a housing assembly 2300 may be provided. The housing assembly 2300 may include a first component 2302 and a second component 2304. The first component 2302 may include a first portion 2306 and a second portion 2308. The second portion 2308 may be disposed above the first portion 2306. The second component 2304 may include a third portion 2310 and a fourth portion 2312. The fourth portion 2312 may be disposed above the third portion 2310. The first component 2302 and the second component 2304 may be assembled such that the first portion 2306 comes in contact with the third portion 2310, and the second portion 2308 coming in contact with the fourth portion 2312. The housing assembly 2300 may include a proximal end 2314 and a distal end 2316. The housing assembly 2300 may define a first bore 2318 extending from the proximal end 2314 until the distal end 2316, in the region of the first portion 2306 and the third portion 2310. A second bore 2320 may be defined, which may extend through the second portion 2308 and the fourth portion 2312. The bore 2318 may be parallel to the second bore 2320. A hollow region may be defined in between the first bore 2318 and the second bore 2320 extending longitudinally. A first slot 2324 may be defined, such that the first slot 2324 extends longitudinally from the edge of the first bore 2318 at the distal end 2316. The first slot 2324 may extend from an outer surface of the first portion 2306 into the first bore 2318. An outer surface of the first portion 2306 from where the first bore 2318 begins at the distal end 2316 defines an externally threaded portion 2326. An outer surface of the second portion 2308 from where the second bore 2320 begins at the distal end 2316 may define an externally threaded portion 2328. A second slot 2330 may be defined at the superior end of the housing assembly 2300 in the region of second portion 2308 and the fourth portion 2312, such that, the second slot 2330 may extend until it reaches the second through hole 2320. A third slot 2332 may be defined at the inferior end of the housing assembly 2300 in the region of the first portion 2306 and the third portion 2310, such that, the third slot 2332 may be extended until it reaches the first bore 2318. A pair of fourth slots 2334 may be defined on lateral sides of the housing assembly 2300. One of the fourth through slot 2334 may be defined in the second portion 2308, and the other fourth through slot 2334 may be defined in the fourth portion 2312. The fourth slots 2334 may oppose each other, and may be exposed to the hollow region of the housing assembly 2300. The first bore 2318 may include several sections that may vary in dimension and diameter along its length. Similarly, the second bore 2320 may include several sections that may vary in dimension and diameter along its length. A protrusion 2336 may be provided on top of the first component 2302 near the distal end 2316, which may define a threaded hole 2338. Plurality of engagement holes may be provided in housing assembly 2300 to facilitate assembly of at least the first component 2302, the second component 2304 and various other components received in the housing assembly 2300. A fifth through slot 2342 and a sixth through slot 2344 may be defined in the third portion 2310. The first slot 2324, the fifth slot 2342 and the sixth slot 2344 may enable translatory movement of various ports that may be used to establish a fluid pathway in the system 110. A seventh slot 2346 may be provided at the superior side of the housing assembly 2300. The seventh slot 2346 may be defined across the second slot 2330. The seventh slot 2346 may facilitate adjustment of position of the adjustment sleeve 1200 with respect to the second arm 1100.

Now reference may be made to FIGS. 24A-24G. The first member 200 may be engaged with the adapter 400. The threaded surface or threaded portion 230 is engaged with the internal thread provided in the adapter 400. Rotation of the adapter 400 may result in rotation of the first member 200. The translatory movement of the first member 200 may result in translatory movement of the adapter 400.

The adapter 400 may be engaged with the power shaft 500. The first portion 506 of the power shaft 500, which may have a polygonal cross section may be received in the bore 422 defined in the adapter 400. At least a portion of the bore 422 may have a polygonal cross section. The first portion 506 may translate along the bore 422. Rotation of the power shaft 500 may result in rotation of the adapter 400. The second portion 508 of the power shaft 500 may engage with a power tool 2404. The second portion 408 of the adapter 400 may engage with a bearing 2402.

The first part 602 of the carriage 600 may be threadably engaged with the second part 604 of the carriage 600. The assembly of first part 602 and the second part 604 may be referred to as carriage 600. The carriage 600 may house the adapter 400 and a portion of the power shaft 500. The bearing 2402 may be received in the bearing engagement groove 610. O-rings 2406 may be received in the O-ring engagement grooves 612. Another O-ring 2408 may be received in the annular groove 644 defined between the first part 602 and the second part 604. A retention ring 2410 may be received in the retention ring engagement groove 614.

The first connecting member 700 may be accommodated on the carriage 600. The protrusion 728 is received in the gap 628 of the carriage 600. The third slot 718 receives the first pillar 622 of the carriage 600. The post 724 may be received by the slot 634 of the carriage 600. Such an engagement of the first connecting member 700 with the carriage 600 may prevent relative translatory motion.

A spring 2412 may be received in the cavity 710 of the first connecting member 700. The stop 800 may be placed over the spring 2412, such that the spring 2412 is also received by the cavity 816 defined in the stop. This may be part of the locking assembly.

A threaded screw 2414 may engage with the threaded hole 712 of the first connecting member 700. An end of the screw 2414 may interface with the first supporting wall 814 of the stop 800. The first slot 714 of the first connecting member 700 may receive the adapting surface 910 of the first arm 900. A pin 2416 may be received through the through hole 912 provided in the first arm 900 and pin holes 720 provided in the first connecting member 700. The pin 2416 may facilitate engagement of the first arm 900 with the first connecting member 700. A pin 2418 may be received in the through hole 810 of the stop 800, such that the pin 2418 may translate along the first slot 2002 of the guide plates 2000.

A threaded adjustment knob 2420 may be engaged with the first arm 900 at its threaded portion 914. The threaded adjustment knob 2420 may be a depth limiting means for configuring a distance between the distal end of the channel created in the tissue by the first member 200 and the external surface 10 of the skin 11.

The first member 200 may be received by or engaged to the second member 300. A portion of the first member 200 is received inside the second bore 332 defined in the second member 300. The arms 318 of the second member 300 may rest over the slots 220 provided in the first member 200. The protrusions 328 provided in the arms 318 may interface with the surface of the slot 220. The engagement protrusions 312 of the second member 300 may be received in the slots 234 of the first member 200. A bearing 2422 may receive the annular protrusion 316 of the second member 300. The bearing 2422 may adapted with the second connecting member 1000. The bearing 2422 may be received in the groove 1008 defined in the second connecting member 1000.

The second connecting member 1000 may be engaged with the second arm 1100. The through slot 1010 of the second connecting member 1000 may receive the second post 1110 of the second arm 1100. Pins 2424 may be passed through the second apertures 1118 of the second arm 1100 and holes 1012 of the second connecting member 1000.

The adjustment sleeve 1200 may be engaged with the second arm 1100. The adjustment sleeve 1200 may be engaged with the threaded portion 1122 of the second arm 1100.

A portion of the first arm 900 may be accommodated in the first bore 1112 and the second bore 1114 of the second arm 1100. The cylindrical shoulder 916 of the first arm 900 may be accommodated in the first bore 1112 and the proximal end 902 of the first arm 900 may extend out of the second bore 1114 of the second arm 1100. The proximal end 902 of the first arm 900 may even extend out of the proximal end 2314 of the second bore 2320 of the housing 2300.

The second arm cap 1400 may be engaged with the second arm 1100. The shaft 1402 of the second arm cap 1400 may be configured to be adapted in the first bore 1112 of the second arm 1100, such that, the head 1404 of the second arm cap 1400 may fit flush with the edge of the second arm 1100 at the distal end 1106 of the second arm 1100.

A clipping spring 2426 or a first compressible means may be accommodated in the first bore 1112 of the second arm 1100 such that, the clipping spring 2426 may be disposed between the step 1120 or intersection of the first bore 1112 and second bore 1114 of the second arm 1100 and the engaging surface 918 of the cylindrical shoulder 916 of the first arm 900. The clipping spring 2426 may enclose a portion of the cylindrical shaft of the first arm 900.

A portion of the second arm 1100 may be received by the stationary arm 1300. The first aperture 1312 of the stationary arm 1300 may accommodate at least a part of the cylindrical portion of the second arm 1100. The link member pin 1700 may be received by the pair of slots 1310 of the stationary arm 1300. The link member pin 1700 may pass through the through hole 1406 of the second arm cap 1400. Another link member pin 1700 may be received by the second aperture 1316 of the stationary arm 1300. A spring 2428 or second compressible means may be accommodated in the first bore 1312 of the stationary arm 1300 such that the spring 2428 may be disposed between the head 1404 of the second arm cap 1400 and the supporting wall 1320 of the stationary arm 1300.

The stationary arm 1300 may be engaged with the external supporting member 1800. The head portion 1328 of the stationary arm 1300 may be received by the elongated housing 1806. An elastic ring 2430 or garter spring or canted coil spring (such as a Bal Seal Canted Coil Spring®) may be received in the annular slot 1848 of the elongated housing 1806, such that the engage elastic ring 2430 surround the neck portion 1326 of the engagement feature 1308 of the stationary arm 1300. A screw 2432 may be received by the threaded hole 1836 of the external supporting member 1800, and a tip of the screw 2432 may interface with the second recess portion 1330 of the stationary arm 1300, such that, the relative motion of the stationary arm 1300 may be prevented with respect to the external supporting member 1800. The counter pressure device 1900 may be engaged with the external supporting member 1800. The counter pressure device 1900 may be received in the first aperture 1808 defined in the external supporting member 1800.

The first paddles 1600 may be operatively engaged with stationary arm 1300 and the second arm 1100. Each of the first paddles 1600 may be disposed on laterally opposing sides of the stationary arm 1300. A link member pin 1700 may be passed through the second aperture 1316 of the stationary arm 1300. One end of the link member pin 1700 may pass through the second through holes 1614 of the link members 1604 disposed on the superior side of the first paddle members 1600. Another end of the link member pin 1700 may pass through the second through holes 1614 of the link members 1604 disposed on the inferior side of the first paddle members 1600.

Another link member pin 1700 may be received by the pair of slots 1310 of the stationary arm 1300. The link member pin 1700 may pass through the through hole 1406 of the second arm cap 1400. The link member 1700 may be engaged with four link members 1500. Two link members 1500 may be disposed on the superior side of the second arm 1100 and the remaining two link members 1500 may be disposed on the inferior side of the second arm 1100. Each of the two link members 1500 disposed on the superior side may be disposed on laterally opposing sides of the second arm 1100. Likewise, each of the two link members 1500 disposed on the inferior side may be disposed on laterally opposing sides of the second arm 1100. One end of the link member pin 1700 may pass through the first through holes 1506 of the link members 1500 disposed on the superior side. Another end of the link member pin 1700 may pass through the first through holes 1506 of the link members 1500 disposed on the inferior side.

Another link member pin 1700 may engage with link members 1500 and the first paddle member 1600 disposed on one of the lateral sides of the stationary arm 1300 or the second arm 1100. One end of the link member pin 1700 may pass through the second through hole 1508 of the link member 1500 disposed on the superior side and first through hole 1612 provided in link member 1604 disposed on the superior side. Another end of the link member pin 1700 may pass through the second through hole 1508 of the link member 1500 disposed on the inferior side and first through hole 1612 provided in the link member 1604 disposed on the inferior side.

Another link member pin 1700 may engage with link members 1500 and the first paddle 1600 disposed on another lateral side of the stationary arm 1300 or the second arm 1100. One end of the link member pin 1700 may pass through the second through hole 1508 of the link member 1500 disposed on the superior side and first through hole 1612 provided in link member 1604 disposed on the superior side. Another end of the link member pin 1700 may pass through the second through hole 1508 of the link member 1500 disposed on the inferior side and first through hole 1612 provided in the link member 1604 disposed on the inferior side. Each of the four link member pins 1700 may receive retention rings in the annular grooves 1702.

The cover member 2100 may receive the first member 200 and the second member 300. The first member 200 and the second member 300 may pass through the bore 2106 of the cover member 2100.

The cover member 2100 may be engaged with the tissue stabilizing member 2200. At least a part of the tissue stabilizing member 2200 may be received by the cover member near the distal end 2102 of the cover member 2100. The pair of pins 2214 may be received in the “U” shaped slot 2110, such that the tissue stabilizing member 2200 may translate in a portion of the “U” shaped slot 2110 that is closed at the distal end 2102. A spring 2434 may be accommodated in the bore 2206 of the tissue stabilizing member 2200. The spring 2434 may be disposed between the flange 2208 of the tissue stabilizing member 2200 and an inwards projecting step or bushing 2436 provided in the cover member 2100.

The bushing 2436 may be received by a stepped inner portion towards the distal end 2102 of the cover member 2100. Another bushing 2436 may be received by another stepped inner portion towards the proximal end 2104 of the cover member 2100.

The first component 2302 and the second component 2304 of the housing 2300 may be engaged such that, outlet port 618 and the flush port 620 may translate in the sixth through slot 2344 and fifth through slot 2342, respectively. The first post 1108 of the second arm 1100 may translate in the second slot 2330 of the housing assembly 2300. A tube 2438 engaged with the aperture 2120 of the cover member 2100 may be retained in the first slot 2324.

The guide plates 2000 may be engaged over the outer surface of the housing assembly 2300 such that, the guide plate 2000 may cover a portion of the fourth slot 2334, such that the pin 2418 received in the through hole 810 of the stop 800 may translate in the first slot 2002 of the guide plates 2000.

A nut 2440 may be engaged with the externally threaded portion 2328 of the housing assembly 2300. The flange 1322 of the stationary arm 1300 may be pressed against the edge of the threaded portion 2328 by the nut 2400, and may prevent the stationary arm 1300 from disengaging from the housing assembly 2300.

Another nut 2442 may be engaged with the externally threaded portion 2326 of the housing assembly 2300. The flange 2124 of the cover member 2100 may be pressed against the edge of the threaded portion 2326 by the nut 2400, and may prevent the cover member 2100 from disengaging from the housing assembly 2300.

A screw 2444 or motion limiting means may be received by the protrusion 2336 of the housing assembly 2300. The screw 2444 may be received in the threaded hole 2338 defined in the protrusion 2336.

Referring to the figures, and more specifically to FIGS. 1A-1E and FIGS. 24F-24Z1, in order to extract a hair follicle, a portion 112 of the system 110 may be moved underneath the skin 10. The portion 112 may include the cover member 2100, the first member 200, the second member 300 and the tissue stabilizing member 2200. At least a part of the portion 112 may be disposed between an inner surface 12 of the skin 10 and the skull 15. The tissue stabilizing member 2200 may be positioned around the tissue that has the target hair follicle 13,14. The tissue stabilizing member 2200 may press against the inner surface 12 of the skin 10, around the tissue that has to hair follicle 13. The counter pressure device 1900 may be disposed opposite to the tissue stabilizing member 2200, and may interface with the external surface 11 of the skin 10. A part of the hair 14 may extend out of the external surface 11. The aperture or opening 1902 provided in the counter pressure device may enable a user to see the hair follicle that is being targeted for extraction. The alignment of the hair follicle 13 may be altered, for example, using piercing members, to align the hair follicle 13 with the longitudinal axis of the first member 200, to avoid transection of hair follicle 13 while it is being extracted.

In order to extract the hair follicle 13 from underneath the skin 10, the first member 200 may have to be rotated about its longitudinal axis while it is translated into the skin 10. The rotation of the first member 200 may be enabled by the power tool 2404. The power tool 2404 imparts torque to the power shaft 500, thereby enabling the power shaft 500 to rotate. The power shaft 500 transfers the torque to the adapter 400, thereby enabling the adapter 400 to rotate. The power shaft 500 may transfer the torque to the adapter 400 even if the adapter moves linearly with respect to the power shaft 500, owing to the configuration of the power shaft 500 and the adapter 400. The adapter 400 transfers the torque to the first member 200, thereby enabling the first member 200 to rotate. Rotation of the first member 200 may result in rotation of the second member 300, owing to engagement between the first member 200 and the second member 300, in which the engagement protrusions 312 of the second member 300 may be received in the slots 234 of the first member 200.

As recited earlier, the first member 200 may have to be translated into the skin from underneath the skin to enable extraction of the hair follicle. In addition to translation of the first member 200, the second member 300 may have to be translated along the axis of the second member 300 or the first member 200 to enabling clipping of tissue that may have been cut by the first member 200.

An operator may insert a portion 112 of the system 110 underneath the skin 10. The extent to which the portion 112 may be inserted may be limited by the nut 2442. The rest of the system 110 and the system 100, which include the counter pressure device 1900 may be exposed to the external surface 11 of the skin. The tissue stabilizing member 2200 may first enter the incision 120, followed by the other parts. The portion 112 is moved underneath the skin 11 such that the tissue stabilizing member 2200 presses around the tissue that has the target hair follicle, from underneath the skin 11, while the counter pressure device 1900 presses the tissue that has the hair follicle from the external surface 11 of the skin, such that the target hair follicle may be disposed between the tissue stabilizing member 2200 and the counter pressure device 1900. It may be noted that, the extent to which the tissue stabilizing member 2200 extends out of the cover member 2100 owing to the tension applied by the spring 2434 over the tissue stabilizing member 2200 may depend on the thickness of the skin 10. The hair follicle that may be disposed between the tissue stabilizing member 2200 and the counter pressure device 1900 may be aligned with the longitudinal axis of the first member 200 before the operator initiates translatory movement of the first member 200. Once, aligned, the operator may initiate the translatory movement of the first member.

The operator may place his thumbs on the concave curvature 1844 defined in the pair of second paddles 1804. The remaining four fingers in each of his hands may be pressed against the first surface 1606 of the first paddles 1602. The operator may begin to apply pressure over the first surface 1606 of the first paddles 1602, so as to move the first paddles 1602 from its initial position towards the second paddles 1804. Movement of the first paddles 1602 may result in movement of the link member pin 1700a (may be referred to as 1700) engaged with the second arm cap 1400 towards another link member pin 1700b (may also be referred to as 1700), while the second arm cap 1400 compresses the spring 2428. The movement of the link member pin 1700a may result in movement or translation of the second arm 1100 in the first direction along its longitudinal axis towards the link member pin 1700b, which may be stationary. The movement of the second arm 1100 may result in the movement of the spring 2426. The movement of the spring 2426 may be achieved as one end of the spring 2426 may be interfacing with the step 1120 of the second arm 1100. The other end of the spring 2426 may in turn apply force over the first engaging surface 918 of the first arm 900, thereby enabling movement of the first arm 900 in the first direction. The first arm 900 may continue moving in the first direction until the threaded adjustment knob 2420 interfaces the surface of the housing assembly 2300 at the housing assembly's 2300 proximal end 2314 (refer FIG. 24K-24L).

Movement of the first arm 900 may result in movement of the first connecting member 700 in the first direction, since it may be connected to the first arm 900 by the pin 2416. Movement of the first connecting member 700 may result in movement of the stop 800, which is received in the first connecting member 700. The pin 2418 received by the stop 800 may translate in the first slot 2002 defined in the guide plate 2000.

Movement of the first arm 900 may also result in movement of the carriage 600 and the adapter 400 in the first direction.

The adapter 400 which may be connected to the first member 200 may result in movement of the first member 200 in the first direction.

The second connecting member 1000 may move in the first direction as a result of the movement of the second arm 1100 in the first direction. The bearing 2422 and the second member 300 connected to the second connecting member 1000 also moves in the first direction.

As recited earlier, the first arm 900 may continue moving in the first direction until the threaded adjustment knob 2420 interfaces the surface of the housing assembly 2300 at the housing assembly's 2300 proximal end 2314. Once the translatory movement of the first arm 900 stops, the components, such as, the first connecting member 700, carriage 600, adapter 400 and the first member 200, which may have translated as a result of translatory movement of the first arm 900 may also stop. The position of the paddles 1602, 1804 at which the first member 200 stops translating in the first direction may be referred to as intermediate position. At this position, the first member 200 may have cut through the tissue that may have the target hair follicle, thereby creating a channel or circular circumferential cut or punch in the tissue, such that at least a portion of the target hair follicle is within the channel (refer FIG. 24M-24N). Further, at this position, pin 2418 may have moved towards the third region 2016 of the first slot 2002 of the guide plate 2000. The second seat 820 of the stop 800 may interface with the cylindrical surface of the adjustment sleeve 1200.

The operator may operate the paddles 1602, 1804 to move beyond the intermediate position to reach a final position. The operator may continue to pull the first paddles 1602 towards second paddles 1804. Movement of the first paddles 1602 may result in further movement of the link member pin 1700 engaged with the second arm cap 1400 towards the other link member pin 1700, while the second arm cap 1400 further compresses the spring 2428. The movement of the link member pin 1700 may result in further movement or translation of the second arm 1100 in the first direction along its longitudinal axis towards the other link member pin 1700. The second connecting member 1000 may further move in the first direction as a result of the movement of the second arm 1100 in the first direction. The bearing 2422 and the second member 300 connected to the second connecting member 1000 also may also move in the first direction. The spring 2426 disposed in the first bore 1112 of the second arm 1100 may be compressed due to the movement of the second arm 1100, while the first arm 900 is restricted from moving further. The movement of the second arm 1100 may result in sliding of the adjustment sleeve 1200 over the second seat 820 of the stop 800 which is being pushed against the adjustment sleeve 1200 by the spring 2412. As the adjustment sleeve 1200 slides past the second seat 820, the spring 2412 expands such that the first seat 818 align or interfaces with the cylindrical surface of the adjustment sleeve 1200 (refer FIG. 240). The instant position may be referred to as locking member extended position. The pin 2418 may interface with a superior edge 2028 of the first slot 2002 in the third region 2016 of the guide plate 2000. The movement of the second arm 1100 and the second member 300 in the first direction may be stopped by a movement restricting feature, such as the screw 2444 engaged in the protrusion 2336 of the housing assembly 2300 (refer FIG. 240-24P). The positions of the paddles 1602 and 1804 at which the movement of the second member 300 in the first direction stops may be referred to as final position.

The translatory movement of the second member 300 while the first member 200 is restricted from moving in the first direction may result in the arms 318 moving into the first bore 206 of the first member 200 through the apertures 222, such that the tips 320 of the arms meet inside the first bore 206, thereby clipping the tissue that may be have the target hair follicle, which may have been cut by the first member (refer FIG. 24Q-24T).

The operator may stop applying force over the paddles 1602, 1804, so that the paddles 1602, 1804 retract from the final position to the initial position.

Upon stopping the application of force over the paddles 1602 and 1804, the spring 2428 may expand, and may apply expansion force on the head 1404 of the second arm cap 1400. As a result of spring 2428 applying expansion force on the second arm cap 1400, the second arm cap 1400 may move in a second direction opposite to the first direction. The second arm cap 1400 may in-turn move the second arm 1100 in the second direction. The adjustment sleeve 1200 may in-turn move in the second direction as a result of the second arm 1100 movement in the second direction. The proximal end of the adjustment sleeve 1200 which may be interfacing the block wall 822 of the stop 800, may move in the second direction such that, the stop 800 may cause the pin 2418 to slide in the third region 2016. The movement of the stop 800 may cause the first connecting member 700, the first arm 900 and the first member 200 to translate or move in the second direction. Hence, the first member 200 and the second member 300 may move simultaneously in the second direction (refer FIG. 24U-24W).

Subsequent movement of the second arm 1100 may cause the pin 2418 to slide into the second region 2014, which has the curved surface 2026 in the guide plate 2000, thereby causing the stop 800 to move downwards or move in an inferior direction. Subsequently, the pin 2118 may slide into the first region 2012, thereby releasing the interface between the proximal end of the adjustment sleeve 1200 and the block wall 822 of the stop 800 (refer FIG. 24X-24Y). The instant position in which various parts of the system 110 that form the locking assembly are in a position that allows second member 300 to translate in the second direction at least relative to the first member 200 may be referred to as locking member retracted position. The position of the first member 200 at this stage may be referred to as first member intermediate position.

Subsequent movement of the adjustment sleeve 1200 may not result in movement of the first connecting member 700, first arm 900 or the first member 200 in the second direction, as the adjustment sleeve 1200 may not be able to push the first connecting member 700 in the second direction. Hence, the first member 200 may not translate in the second direction, while the second member 300 translates in the second direction. This may cause the arms 318 to retract out of the first bore 206, and the arms 318 or the protrusions 328 of the arms 318 may rest on the slots 220 of the first member 200. The second member 300 may translate in the second direction, while the first arm 200 is stationary, with respect to the translatory movement, until the adjustment sleeve 1200 interfaces with the pair of arms 726 of the first connecting member 700. The position of the second member 300 at this stage may be referred to as second member retracted position (refer FIG. 24Z-24Z1). Once the interface is established, the first arm 900 and the first member 200 may begins to translate along with the second arm 1100 and the second member 300, until the paddles 1602 and 1804 reaches the initial position.

The components, such as, guide plate 2000, first connecting member 700, stop 800, spring 2412, pin 2418 and adjustment sleeve 1200, which may enable locking so as to enable simultaneous translatory movement of the first member 200 and the second member 300 in the second direction after the movement in the first direction is terminated, may be referred to as locking assembly. The position of the locking assembly when the adjustment sleeve 1200 interfaces with the block wall 822 may be referred to as locking member or locking assembly extended position. The position of the locking assembly when the adjustment sleeve 1200 interfaces with the second seat 820 may be referred to as locking member or locking assembly retracted position.

A depth limiting means or the knob 2420 may be adjusted to either be position closer to the proximal end 2314 of the housing assembly 2300 to control the extent to which the first member 200 translates in the first direction. Controlling the extent to which the first member 200 translates in the first direction may in turn enable controlling the depth of cut made by the first member 200 into the tissue. Hence, the distance between the distal end of the channel cut into the tissue and the external surface of the skin may be controlled. The operator may make such adjustment based on the desired depth of cut or thickness of the skin.

A motion limiting means or the screw 2444 received by the protrusion 2336 of the housing assembly 2300 may be adjusted to configure the extent to which the second member 300 or second means may be translated in the first direction.

The rotation of the first member 200 may be terminated, operably or automatically, after the first member 200 has terminated its movement in the first direction.

Alternatively, the rotation of the first member 200 and the second member 300 may be terminated, operably or automatically, before initiating the translatory movement of the first member 200 and the second member 300 in the second direction.

Alternatively, the rotation of the first member 200 may be terminated, operably or automatically, after the first member 200 has terminated its movement in the first direction. The rotation of the first member 200 and the second member 300 may be initiated after the second member 300 has terminated its movement in the first direction.

Alternatively, the first member 200 and the second member 300 may not be rotated, and the first member 200 and second member 300 may only be translated to enable cutting and clipping, or to enable extracting of the target tissue.

Alternatively, the first member 200 and the second member 300 may be rotated throughout the process of extracting the tissue from underneath the skin.

The flush port 620 may be generally closed. The flush port 620 may be opened to receive fluid into the chamber 616. Generally, while extracting the hair follicle, fluid may be passed into the inlet port 2438, while suction may be applied at the outlet port 618. The fluid received through the inlet port 2438 may pass through the channel 2118 provided in the cover member 2100, and through the channel provided in the bushing 2436 to enter the first bore 206 provided in the first member 200. The fluid may enter the first bore 206 through one or more openings 222 or opening of the first bore 206 at the distal end 210. The fluid may enter the first bore 206 due to suction applied at the outlet port 618. The fluid may carry the hair follicle that may be disposed in the first bore 206 after it has been cored and clipped, towards the exit port. The hair follicle along with fluid may travel through the first bore 206 and enter the adapter 400. The hair follicle along with fluid may exit the adapter 400 and enter the chamber 616 through apertures 418. Eventually the hair follicle along with fluid may exit the system 110 through the outlet port 618.

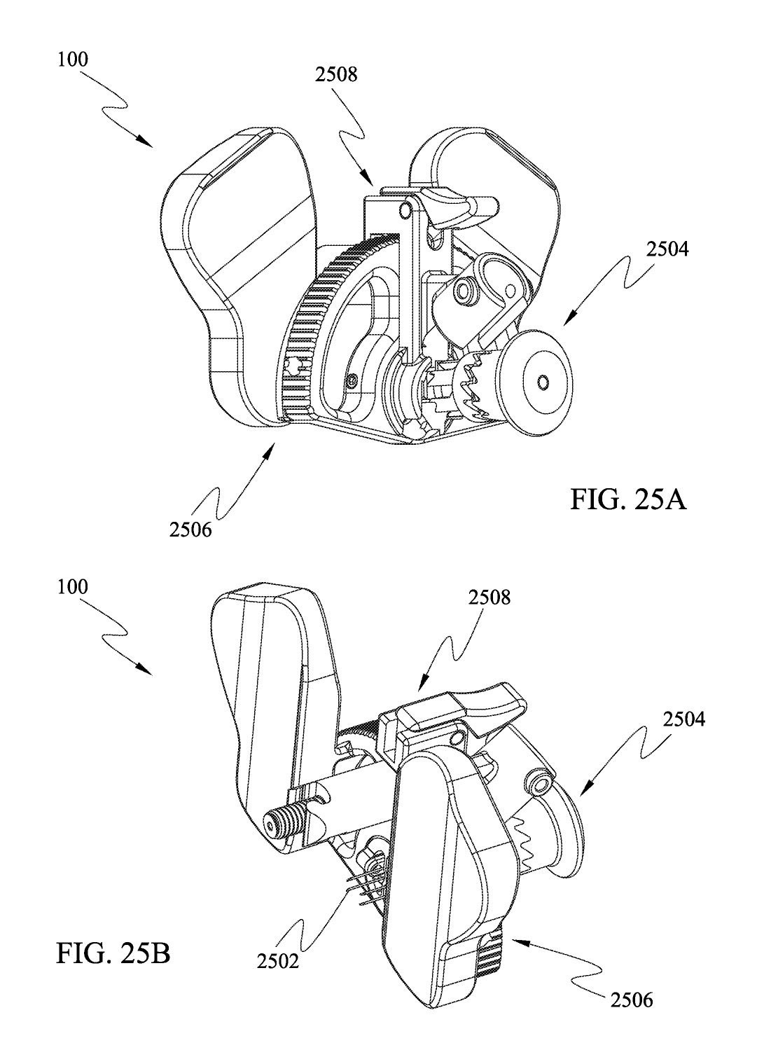

Referring to FIG. 25A-25B, the system 100 may further include a plurality of piercing members 2502, an assembly 2504 for operating the piercing members 2502, a guide member 2506 or the external supporting member 1800 and an arm 2508. The piercing members 2502 may be engaged to a shaft present in the assembly 2504. The assembly 2504 may be configured to operate the piercing members 2502 to translate in a first direction towards the external surface 11 of the skin 10. The translatory motion of the piercing members 2502 in the first direction may allow the piercing members 2502 to pierce into the skin 10 through the external surface 11 of the skin 10. The piercing members 2502 may assume an extended position after traversing in the first direction. The hair follicle to be extracted may be disposed between the plurality of the piercing members 2502 such that the piercing members 2502 may align the hair follicle by pivoting the piercing members 2502. Once the hair follicle aligns with the first member 200, a portion of the hair follicle may be extracted from underneath the skin 10 by the device 110. Upon completion of extraction, the piercing members 2502 may be operated by the assembly 2504 to translate in a second direction to assume a retracted position.

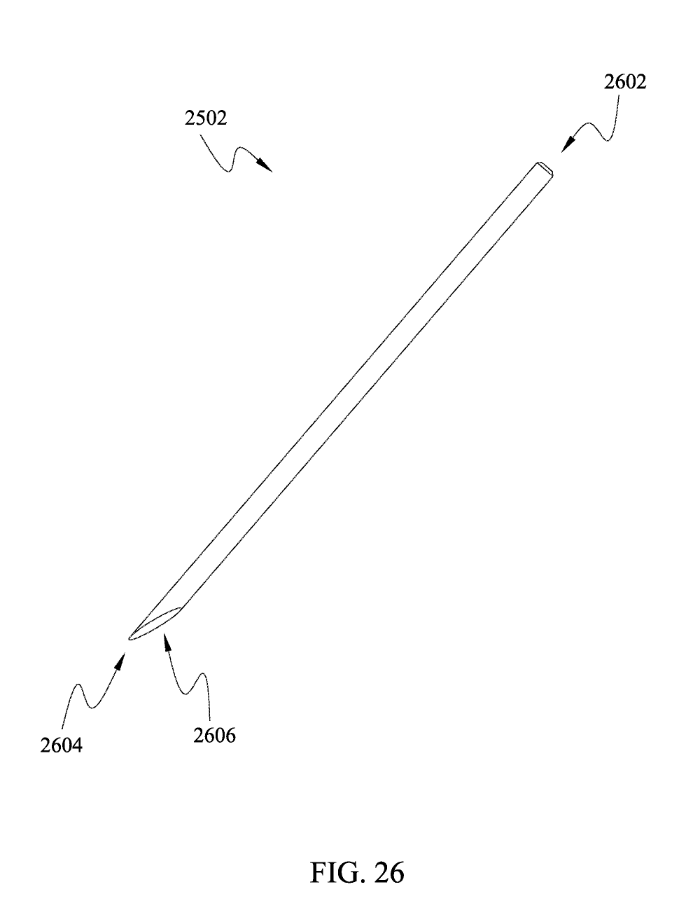

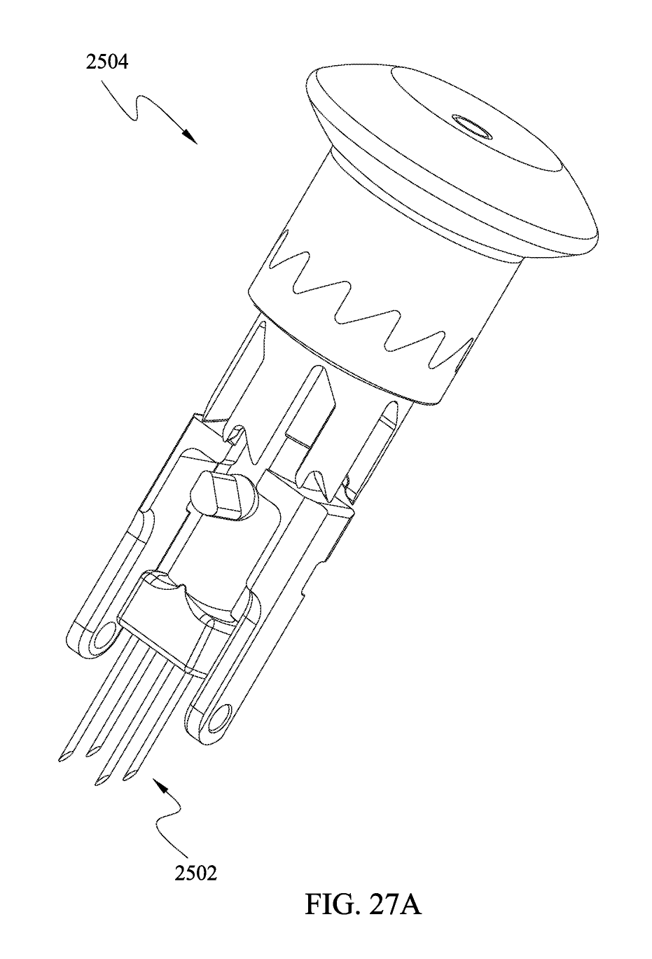

Referring to FIG. 26, the piercing member 2502 or piercing means may include a proximal end 2602 and a distal end 2604. The piercing member 2502 may be a needle defining a pointing distal end 2604. The pointing distal end 2604 may be achieved by providing a beveled configuration 2606 towards the distal end 2604. The pointed piercing member 2502 may enable the piercing member 2502 to be pierced through tissue. The piercing members 2502 may be engaged to a part of the assembly 2504 at the proximal end 2602 of the piercing members 2502.

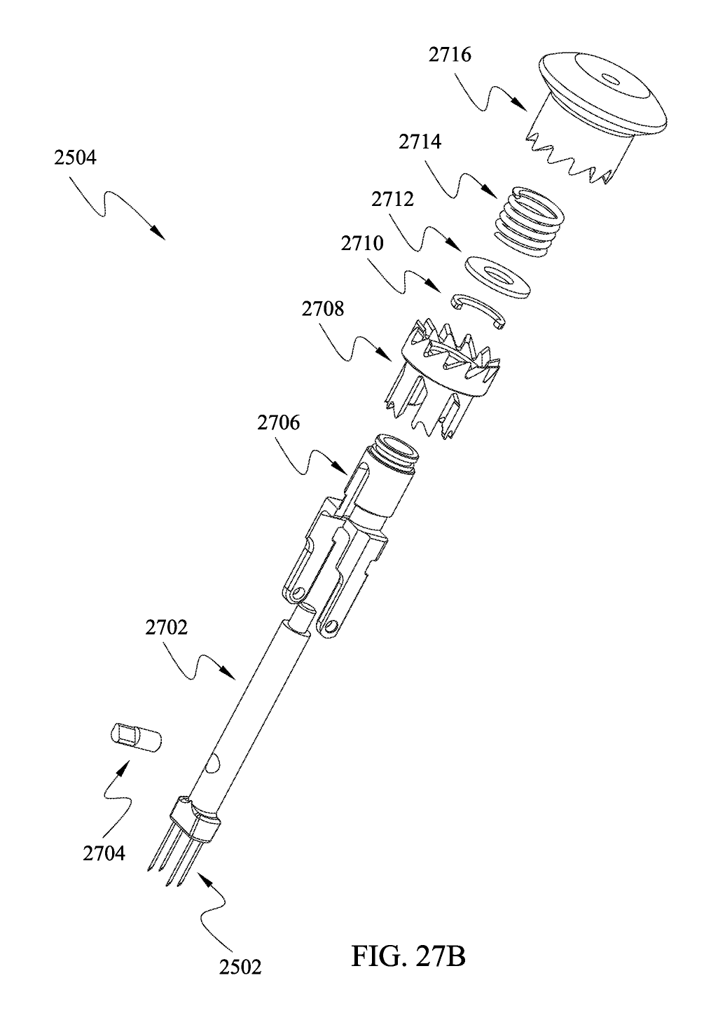

Referring to FIG. 27A-27B, the assembly 2504 or control means 2504 may be employed for receiving the piercing members 2502 and operating the piercing members 2502. The assembly 2504 may include a shaft 2702, an engagement member 2704 or engagement means, a pivot enabling member 2706 or pivot enabling means, a second engagement member 2708, a first ring 2710, a second ring 2712 or washer, a compression member 2714 and a cap 2716.

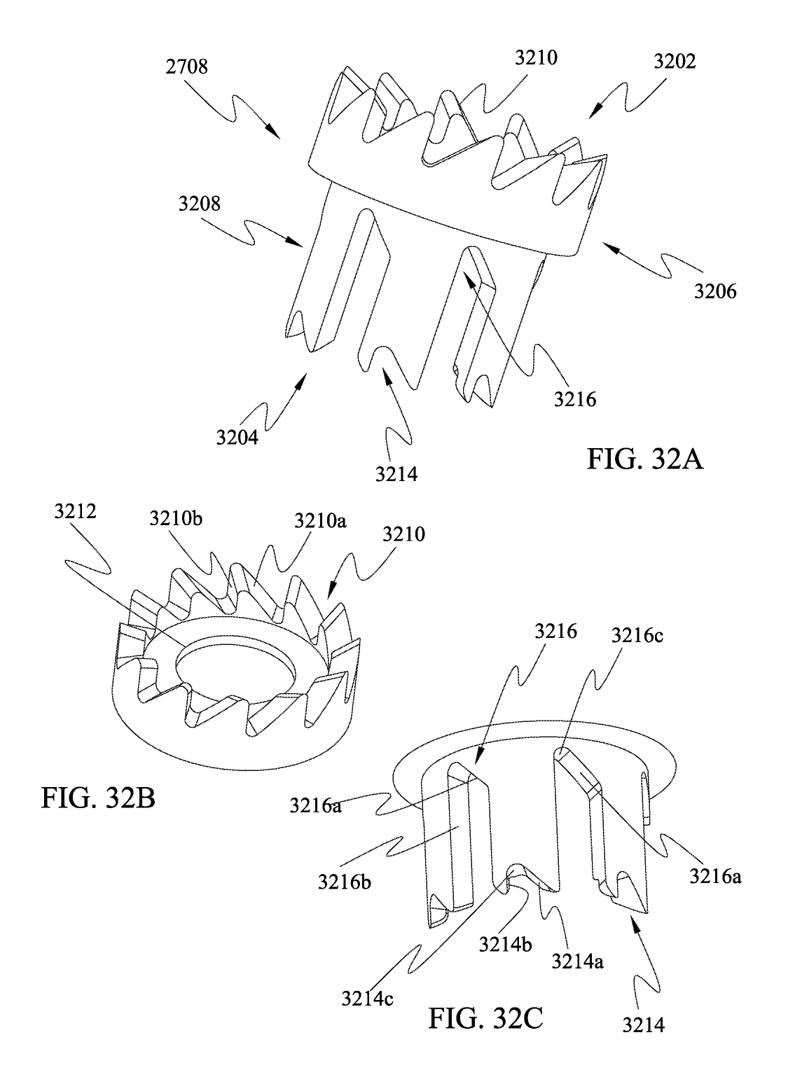

The piercing members 2502 may be made to pierce through the external surface 11 of the skin 10, by pressing the cap 2716. Pressing of the cap 2716 may push the shaft 2702 towards the skin 10, and may also enable partial rotation of the second engagement member 2708, such that the engagement member 2704 is aligned with one of a plurality of extended position lock slot 3214 (illustrated in FIG. 32A) defined in the second engagement member 2708. Releasing of the cap 2716 may enable the shaft 2702 to translate in a direction opposite to the direction in which it translated earlier. The translatory movement of the shaft 2702 will be stopped by engagement of the engagement member 2704 with the second engagement member 2708 at the extended position lock slot 3214, thereby enabling the piercing members 2502 to assume an extended position.

The piercing members 2502 may be made to retract out of the tissue from the extended position to a retracted position, by pressing the cap 2716. Pressing of the cap 2716 pushes the shaft 2702 further, thereby disengaging the engagement member 2704 from the second engagement member 2708. Pressing of the cap 2716 may also enable partial rotation of the second engagement member 2708, such that the engagement member 2704 is now aligned with one of the retracted position lock slot 3216 (illustrated in FIG. 32A) defined in the second engagement member 2708. Releasing of the cap 2716 will enable the piercing members 2502, along with the shaft 2702 to translate out of the tissue. The translatory movement of the shaft 2702 may be stopped by engagement of the engagement member 2704 with the second engagement member 2708 at one of the plurality of retracted position lock slot 3216, thereby enabling the piercing members 2502 to assume the retracted position.

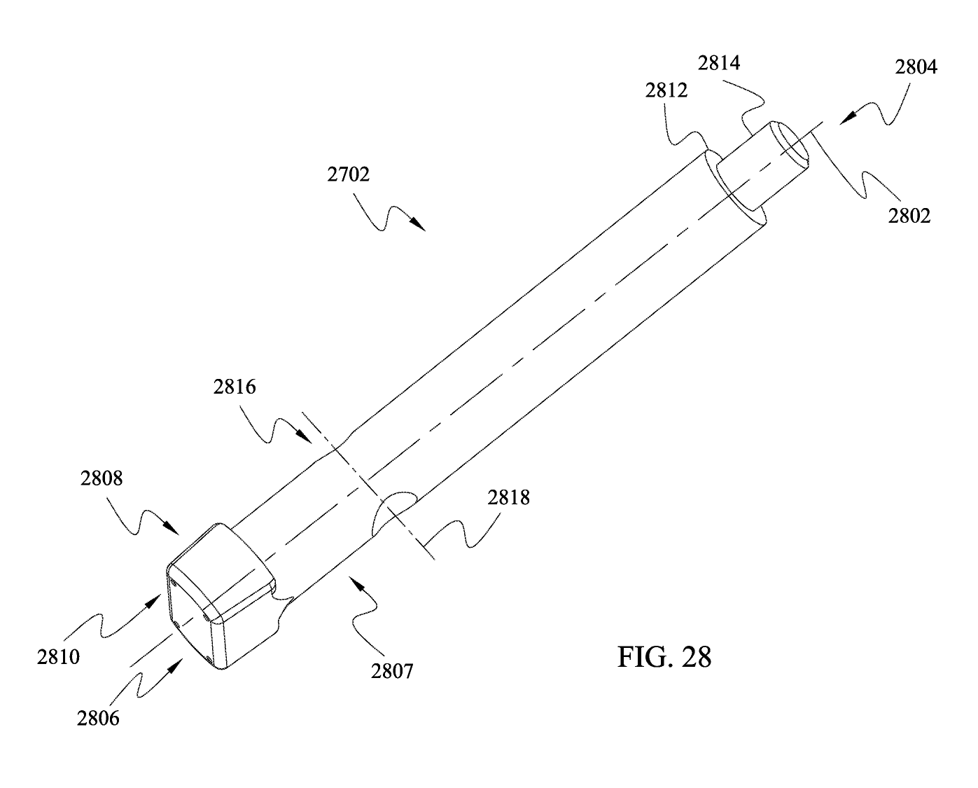

Referring to FIG. 28, a substantial portion of the shaft 2702 may define a cylindrical cross section along its longitudinal axis 2802. The shaft 2702 may be constructed to have any other suitable cross section, such as a polygonal cross section. The shaft 2702 may include a proximal end 2804 and a distal end 2806. The shaft 2702 may include a base 2808 at the distal end 2806. The base 2808 may be such that, cross sectional area of the base 2808 may be greater than the cross sectional area of a portion 2807 of the shaft 2702 that is present immediately after the base 2808. In an embodiment, the base 2808 may be such that, cross sectional area of the base 2808 may be greater than the cross sectional area of rest of the shaft 2702 about the longitudinal axis 2802. The above mentioned exemplary configuration may enable the assembly 2504 to have a relatively smaller footprint, while enabling the piercing members to be spread about a larger cross sectional area. The base 2808 of the shaft 2702 may comprise a plurality of first apertures 2810 extending from the distal end 2806 into the shaft 2702 parallel to the longitudinal axis 2802. The apertures 2810 may be configured to accommodate the piercing members 2502 such that a portion of the piercing members 2502 may be accommodated in the first apertures 2810, and a remaining portion which includes the pointed distal end 2604 may protrude out of the shaft 2702.

In an embodiment, the base 2808 and the piercing members 2502 may be configured such that, one or more piercing members 2502 diverge as they extend away from the base 2808.

In another embodiment, the base 2808 and the piercing members 2502 may be configured such that, one or more piercing members 2502 converge as they intersect with the base 2808.

The shaft 2702 may define a shoulder 2812 towards the proximal end 2804. Further, the shaft may include a neck 2814, which may extend from the shoulder 2812 towards the proximal end 2804 of the shaft 2702. The neck 2814 may be cylindrical in shape, or may have any other suitable shape. The neck 2814 may have a diameter smaller than the diameter of the portion of the shaft 2702 immediately preceding it.

The shaft 2702 may define a second aperture 2816, comprising a longitudinal axis 2818. The axis 2818 of the second aperture 2816 and the longitudinal axis 2802 of the shaft 2702 may intersect with each other. The axis 2818 of the second aperture 2816 and the longitudinal axis 2802 of the shaft 2702 may be orthogonal to each other. The second aperture 2816 may be configured to accommodate the engagement member 2704.

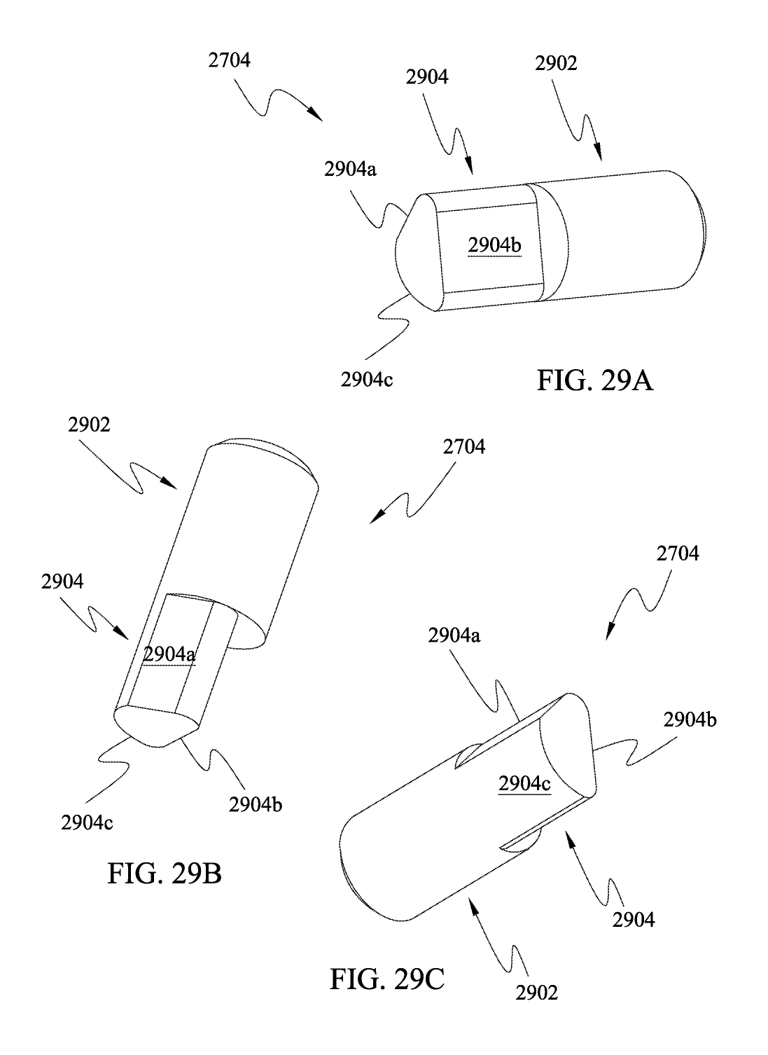

Referring to FIGS. 29A-29C, the engagement member 2704 may include an insertion portion 2902 and a protrusion portion 2904. The insertion portion 2902 of the engagement member 2704 may be configured to be accommodated within the second aperture 2816 defined in the shaft 2702. The insertion portion 2902 of the engagement member 2704 may have a cylindrical cross section. Alternatively, the cross section of the insertion portion 2902 may be polygonal, and the second aperture 2816 may define a complimentary cross section. The insertion portion 2902 may be accommodated within the second aperture 2816 by means of a friction fit, press fit or interference fit, among other means of engagement.

The protrusion portion 2904 of the engagement member 2704 may be configured such that when the insertion portion 2902 is accommodated within the second aperture 2816, the protrusion portion 2904 protrudes out of the shaft 2702. The shape of the protrusion portion 2904 may be such that it engages with any one of the extended position lock slot 3214 and the retracted position lock slot 3216 to enable the piercing members 2502 to assume the extend position or retracted position.

The protrusion portion 2904 may extend from the insertion portion 2902. The protrusion portion 2904 may have “V” or “U” shaped cross section. The “V” or “U” shaped configuration may be asymmetrical. The protrusion portion 2904 may include a first surface 2904a, a second surface 2904b and a third surface 2904c. The first surface 2904a and the second surface 2904b may intersect to define the “V” shaped cross section. Further, the third surface 2904c may intersect the first surface 2904a and the second surface 2904b. One of the more intersections between the first surface 2904a, the second surface 2904b and the third surface 2904c may define curved, transitory or smooth edges or angles. Alternatively, one or more intersections between the first surface 2904a, the second surface 2904b and the third surface 2904c may define hard angles or sharp edges.

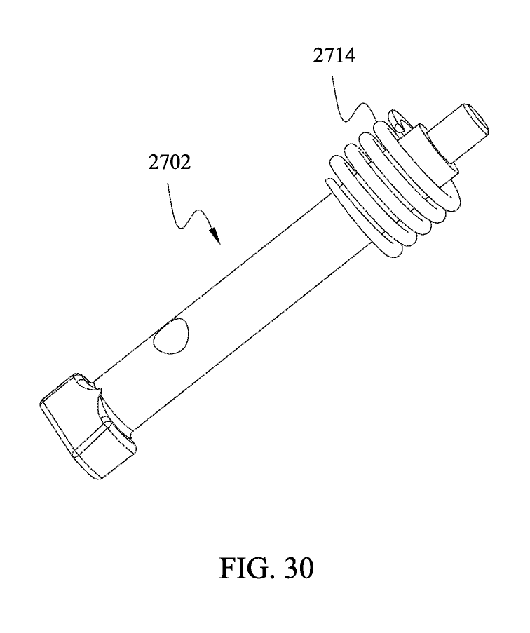

Referring to FIG. 30, the compression member 2714 may be a helical spring 2714. The compression member 2714 may be dimensioned such that it may encircle a portion of the shaft 2702 between the proximal end 2804 and the distal end 2806 of the shaft 2702. The shaft 2702 may translate along a longitudinal axis of the compression member 2714. The longitudinal axis of the compression member 2714 and the longitudinal axis of the shaft 2702 may coincide. The compression member 2714 may enable the shaft 2702 to retract along the longitudinal axis 2802 of the shaft 2702.

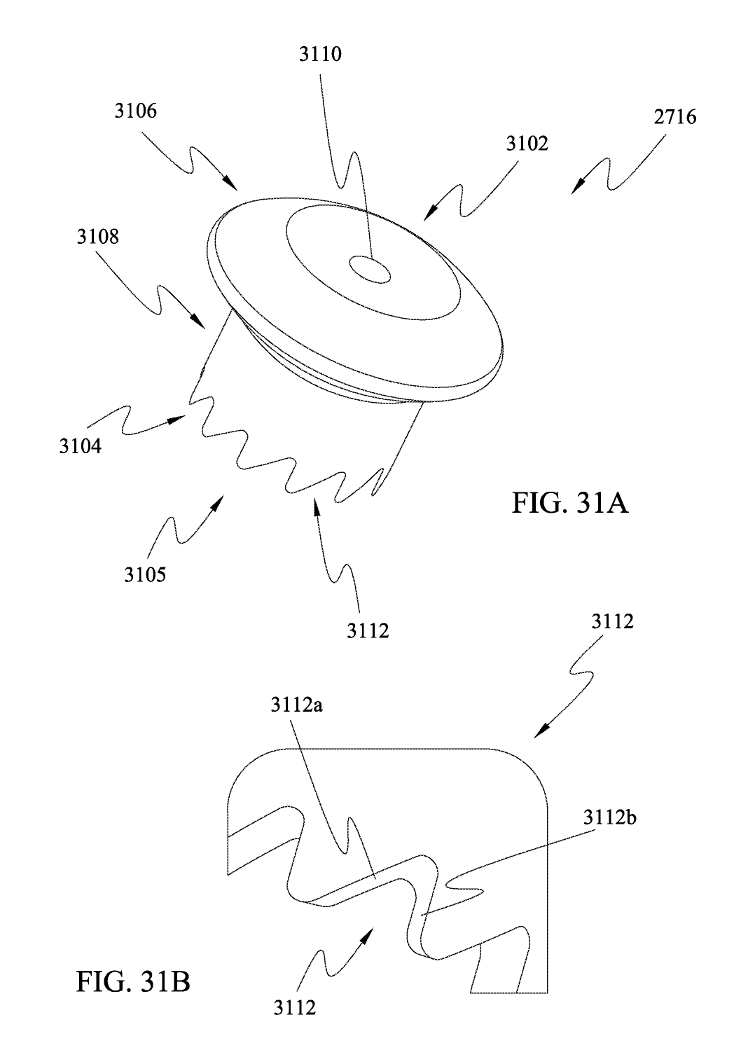

The shaft 2702 may be rigidly engaged with the cap 2716. The cap 2716 may be configured to receive the neck 2814 of the shaft 2702. Referring to FIGS. 31A-31B, the cap 2716 may include a proximal end 3102 and a distal end 3104. The cap 2716 may include a lid portion 3106 and a cover portion 3108. The lid portion 3106 may be provided towards the proximal end 3102 of the cap 2716. The cover portion 3108 may extend from the lid portion 3106 towards the distal end 3104, such that an empty space 3105 is defined by the lid portion 3106 and the cover portion 3108. The lid portion 3106 may define an aperture 3110. The aperture 3110 may extend from an inside surface of the lid portion 3106 towards an outside surface of the lid portion 3106. The aperture 3110 may be configured to accommodate the neck 2814 of the shaft 2702. The neck 2814 may be engaged by press or interference fitting with the cap 2716, among other means of engagement.

The cover portion 3108 may define teeth 3112 extending towards the distal end 3104 of the cap 2716. Each tooth 3112 may be formed by a first surface 3112a intersecting with a second surface 3112b to define an asymmetrical “V” shaped tooth. The first surface 3112a may be beveled. The teeth 3112 defined in the cap 2716 may interface with corresponding teeth defined in the second engagement member 2708.

Referring to FIGS. 32A-32C, the second engagement member 2708 may include a proximal end 3202 and a distal end 3204. The second engagement member 2708 may include a head 3206 and a plurality of second engagement member arms 3208. The head 3206 may be provided towards the proximal end 3202 of the second engagement member 2708. The second engagement member arms 3208 may extend from the head 3206 towards the distal end 3204 of the second engagement member 2708. The head 3206 may define teeth 3210 complimentary to the teeth 3112 defined in the cap 2716. Each tooth 3210 may be formed by a first surface 3210a intersecting with a second surface 3210b to define an asymmetrical “V” shaped tooth. The first surface 3210a may be beveled. The teeth 3210 defined in the second engagement member 2708 may interface with corresponding teeth 3112 defined in the cap 2716.

The head 3206 may define a ring 3212 surrounded by the teeth 3210. The teeth 3210 may extend in a first direction towards the proximal end 3202 from the ring 3212, while the second engagement member arms 3208 may extend in a second direction towards the distal end 3204 from the ring 3212. Each second engagement member arms 3208 may define an extended position lock slot 3214, while adjacent second engagement member arms 3208 may define a retracted position lock slot 3216. Each of the extended position lock slots 3214 and the retracted position lock slot 3216 may be complimentary to the shape of the protrusion portion 2904, such that each of the slots 3214, 3216 is configured to receive the protrusion portion 2904 of the engagement member 2704.

Each of the slots 3214 may have “V” or “U” shaped cross section. The “V” or “U” shaped configuration may be asymmetrical. Each slot 3214 may be defined by a first surface 3214a and a second surface 3214b. The first surface 3214a may be beveled. The first surface 3214a and the second surface 3214b may intersect to define the “V” shaped cross section. The intersection between the first surface 3214a and the second surface 3214b may define curved, transitory or smooth edges or angles. Alternatively, the intersection between the first surface 3214a and the second surface 3214b may define hard angles or sharp edges. Similarly, slots 3216 may be defined by first surface 3216a and a second surface 3216b. The intersection 3216c of the “V” configuration in slot 3216 is closer to the proximal end 3202 or ring 3212 when compared with intersection 3214c of the “V” configuration in slot 3214. The intersection 3214c of the “V” configuration in slot 3214 is closer to the distal end 3204 when compared with intersection 3216c of the “V” configuration in slot 3216.

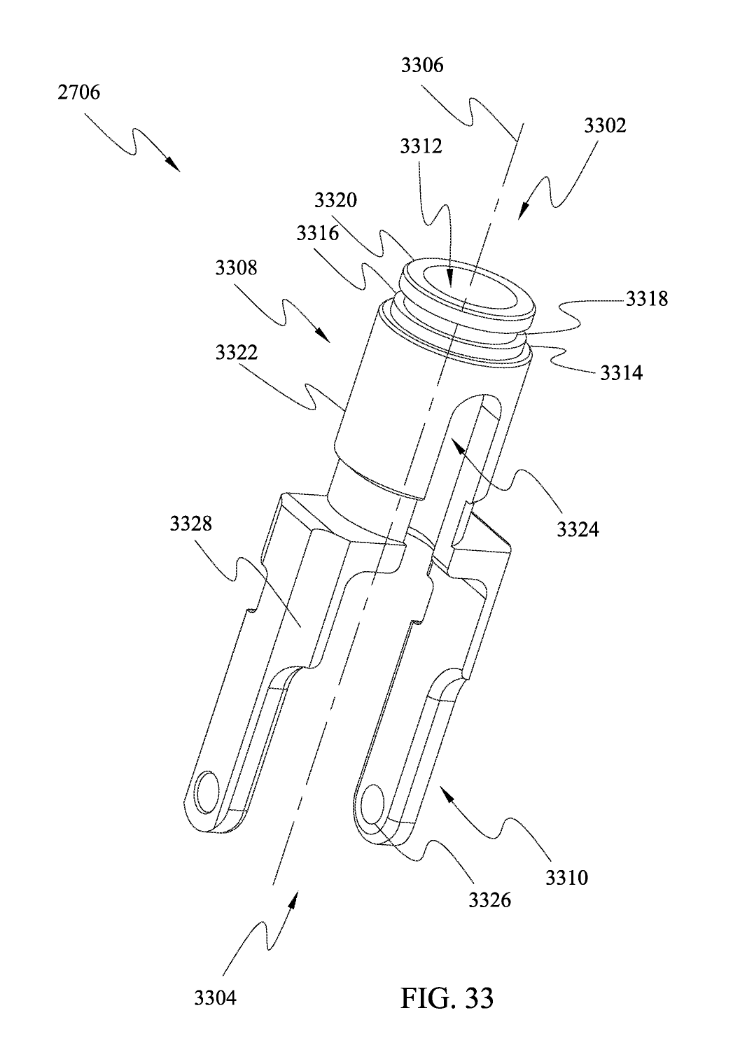

Referring to FIG. 33, the pivot enabling member 2706 may have a proximal end 3302, a distal end 3304 and a longitudinal axis 3306. The pivot enabling member 2706 may include a pivot enabling member head 3308 and a pair of pivot enabling member arms 3310. The pivot enabling member head 3308 may define a through hole 3312 configured to allow the shaft 2702 to translate along the axis 3306. The pivot enabling member head 3308 may include a shoulder 3314, a first neck 3316, a second neck 3318 and a head 3320. The first neck 3316 may extend from the shoulder 3314, the second neck 3318 may extend from the first neck 3316, and the head 3320 may extend from the second neck 3318. The first neck 3316 may have a diameter that is smaller than the diameter of the shoulder 3314. The second neck 3318 may have a diameter that is smaller than the diameter of the first neck 3316. The head 3320 may have a diameter that is larger than the diameter of the second neck 3318. The first ring 2710, which may be a retention ring, may be engaged around the second neck 3318. The second ring 2712 may be disposed on top of the head 3320.

The pivot enabling member head 3308 may have a cylindrical body 3322 extending along the axis 3306. The cylindrical body 3322 may define a slot 3324 extending along the axis 3306, such that the slot 3324 has an open end towards the distal end 3304.

The arms 3310 may extend from the pivot enabling member head 3308 towards the distal end 3304. Each of the arms 3310 may define apertures 3326 configured to engage with pivot pins, thereby enabling the pivot enabling member 2706 to pivot.

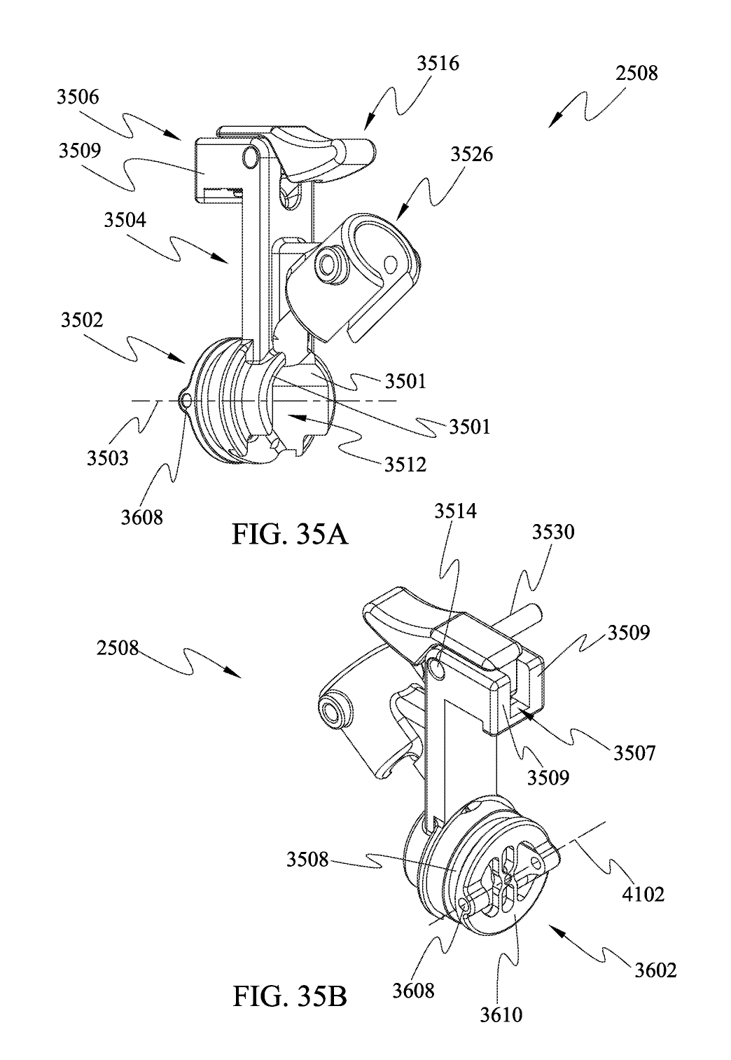

Each of the arms 3310 may define beveled surface 3328 that compliment with beveled surface that may be provided in the cylindrical body 3502 (illustrated in FIG. 35A) of the arm 2508. The beveled surfaces of the pivot enabling member 2706 may mate with corresponding beveled surfaces of the arm 2508, so that the assembly 2504 may be held along an axis where such a fit is established, and the longitudinal axis of the shaft is in line with the axis of the first member 200 provided in the device 110.

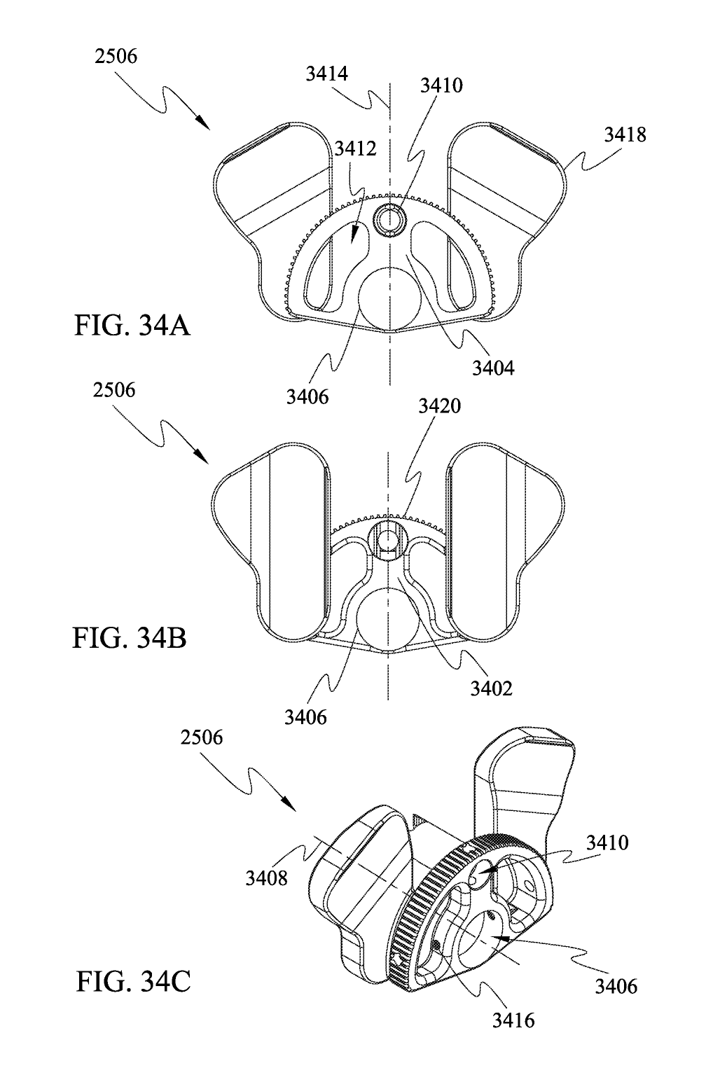

Referring to FIGS. 34A-34C, the guide member 2506, the external supporting member 1800 or alignment means may include an arc shaped element. The guide member 2506 may include a first surface 3402 and a second surface 3404. The first surface 3402 of the guide member 2506 may face the external surface 11 of the skin 10, while in use. The second surface 3404 may face away from the external surface 11 of the skin 10, when engaged with the device 110. An aperture 3406 (through hole) may be defined in the guide member 2506. The aperture 3406 may extend from the first surface 3402 to the second surface 3404. The longitudinal axis of the aperture 3406 may be referred to as rotational axis 3408. The aperture 3406 may be configured to allow the shaft 2702 having the piercing members 2502 to translate along the rotational axis 3408 or the longitudinal axis of the shaft 2702. The aperture 3406 may be configured to allow the shaft 2702 having the piercing members 2502 to rotate about the rotation axis 3408.

The guide member 2506 may further include an additional aperture 3410, which may enable the system 2500 or guide member 2506 to be engaged with the device 110 configured to extract hair follicles from underneath the skin 10.

The guide member 2506 may further define a pair of apertures 3412 defined in either sides of a vertical axis 3414 of the apertures 3406. A plurality of holes 3416 extending from the surface defining the aperture 3406 into apertures 3412 and 3410 may be defined. The longitudinal axis of the holes 3416 may be perpendicular to the longitudinal axis of the aperture 3406. Further, a pair of paddles 3418 may be provided in the guide member 2506.

The guide member 2506 may comprise a plurality of engagement shapes 3420 distributed along the rim of the arc. The engagement shapes 3420 may be configured such that each engagement shape 3420 is equidistant from the rotational axis 3408. Each engagement shape 3420 may be in the form of a groove configured to accommodate a complementing feature.

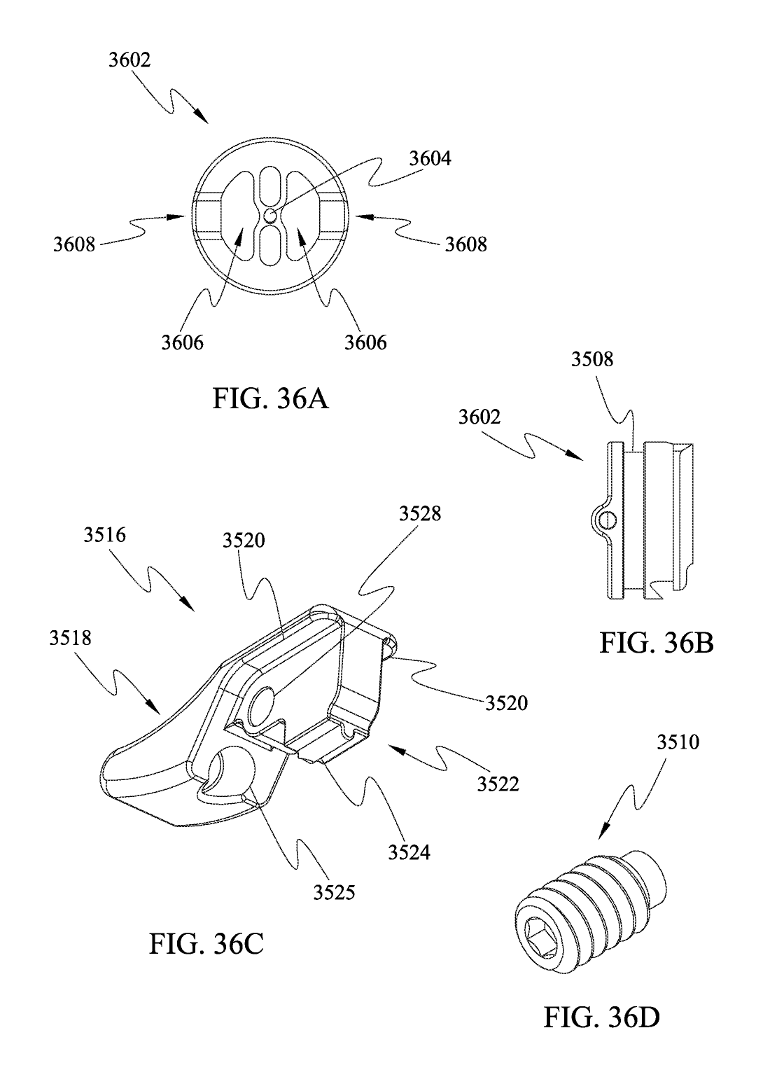

Referring to FIGS. 35A-36D, the arm 2508 may include a cylindrical body 3502, a pillar 3504 and an extending member 3506. The arm 2508 may be configured to be operable to at least partially rotate about the rotational axis 3408 of the guide member 2506.

The cylindrical body 3502 may have a longitudinal axis 3503 which may coincide with the rotational axis 3408. The cylindrical body 3502 may include a groove 3508 on its external surface, such that the groove 3508 aligns with the holes 3416 of the guide member 2506, when assembled. The arm 2508 may be engaged with the guide member 2506 by means of engagement screws 3510 which may be passed through the holes 3416 defined in the guide member 2506, such that a part of the engagement screws 3510 are received by the groove 3508.

The cylindrical body 3502 may define an opening 3512 at one of its ends, so that the shaft 2702 may be received through the opening 3512. At a second end of the cylindrical body 3502 a counter pressure device 3602 or the counter pressure device 1900 may be provided.

The inner surface of the cylindrical body 3502 may define beveled surfaces 3501. The beveled surfaces 3501 may coincide with the beveled surfaces 3328 provided in the pivot enabling member arms 3310. The beveled surfaces 3328 and 3501 may mate with each other by means of friction fit, so that the assembly 2504 may be coaxial with the first member 200 of the device 110 that may be employed for extracting hair follicle from underneath the external surface 11 of the skin 10.

The pillar 3504 may extend from the outer surface of the cylindrical body 3502. The pillar 3504 may extend perpendicularly to the longitudinal axis 3503 of the cylindrical body 3502. The extending member 3506 may extend from the end of the pillar 3504 which may be away from the cylindrical body 3502. The extending member 3506 may define an opening 3507 at the center, such that, two arms 3509 may be defined on each side of the opening 3507

The extending member 3506 may further define a pair of pivot openings 3514 on each of its two arms 3509. The pivot opening 3514 may be defined in the portion of the arms 3509 which is closer to the pillar 3504.

The arm 2508 may further include a latch 3516. The latch 3516 may be shaped to define a handle 3518, a shoulder 3520 and a beak shaped feature 3522. The beak shaped feature 3522 may extend from the shoulder 3520. Inferior to the shoulder 3520, a protruded portion 3524 may be defined. The protruded portion 3524 may compliment the engagement shapes 3420 or grooves provided in the guide member 2506. The latch 3516 may define a hole 3528 that may receive a pivot pin 3530, which may facilitate the latch 3516 to pivot about the longitudinal axis of the pivot pin 3530 or hole 3528. The latch 3516 may further define a spring accommodation hole 3525 configured to receive a spring, such that the spring pushes the latch 3516 into a position that engages the protruded portion 3524 with the guide member 2506.

The arm 2508 may further include a collar 3526 which may be configured to receive an image capturing system.

The counter pressure device 3602 may be a circular plate with a targeting aperture 3604 at the center of the plate. The counter pressure device 3602 may further define piercing member accommodation opening 3606 defined on both sides of the targeting opening 3604. The piercing members 2502 may translate to-and-fro through the piercing member accommodation opening 3606. Further, the piercing member accommodation opening 3606 may define an oblong shape so that the piercing members 2502 may pivot while the piercing members 2502 have extended through the piercing member accommodation opening 3606 or the counter pressure device 3602.

The counter pressure device 3602 may define a pair of pivot holes 3608. The longitudinal axis of the pivot holes 3608 may be perpendicular to the longitudinal axis of the targeting aperture 3604. The pivot holes 3608 may be defined such that a surface 3610 of the counter pressure device 3602 that interfaces with the skin is flush, without protrusions.

The counter pressure device 3602 may apply pressure over or around the tissue comprising hair follicle when it is held against the external surface 11 of the skin 10.

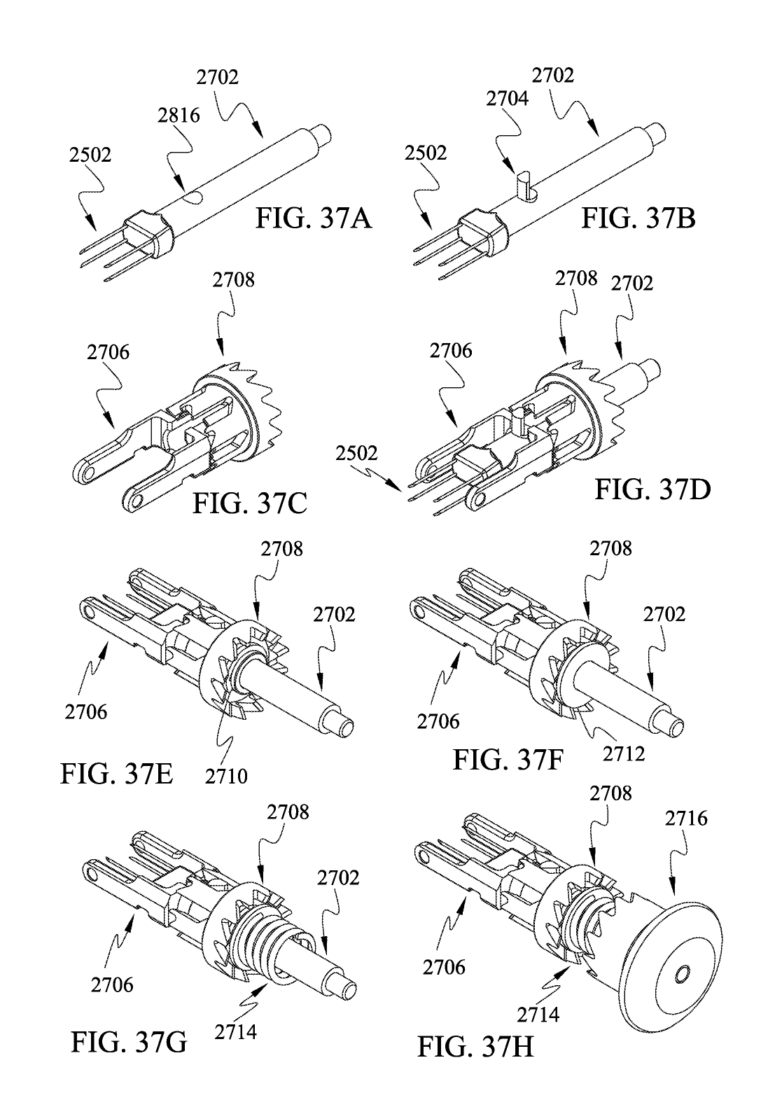

Referring to FIGS. 37A-37H, the engagement member 2704 may be received in the second aperture 2816 defined in the shaft 2702. The pivot enabling member 2706 may be engaged with the second engagement member 2708, such that shoulder 3314 presses against a bottom portion of the ring 3212 of the second engagement member 2708, and the second neck 3318 extends over the ring 3212. The first ring 2710 may be engaged around the second neck 3318, thereby preventing the second engagement member 2708 from disengaging from the pivot enabling member 2706. The second ring 2712 may be placed over the head 3320 of the pivot enabling member 2706. The compression member 2714 may be placed over the second ring 2712. The proximal end 2804 of the shaft 2702 may be received through the pivot enabling member 2706, second engagement member 2708, the first ring 2710, the second ring 2712, and the compression member 2714. The shaft 2702 may be engaged with the cap 2716, such that the neck 2814 of the shaft 2702 is received in the aperture 3110 defined in the cap 2716. The shaft 2702 may be engaged with the cap 2716, such that relative movement between the shaft 2702 and the cap 2716 is prevented, once they are engaged together.

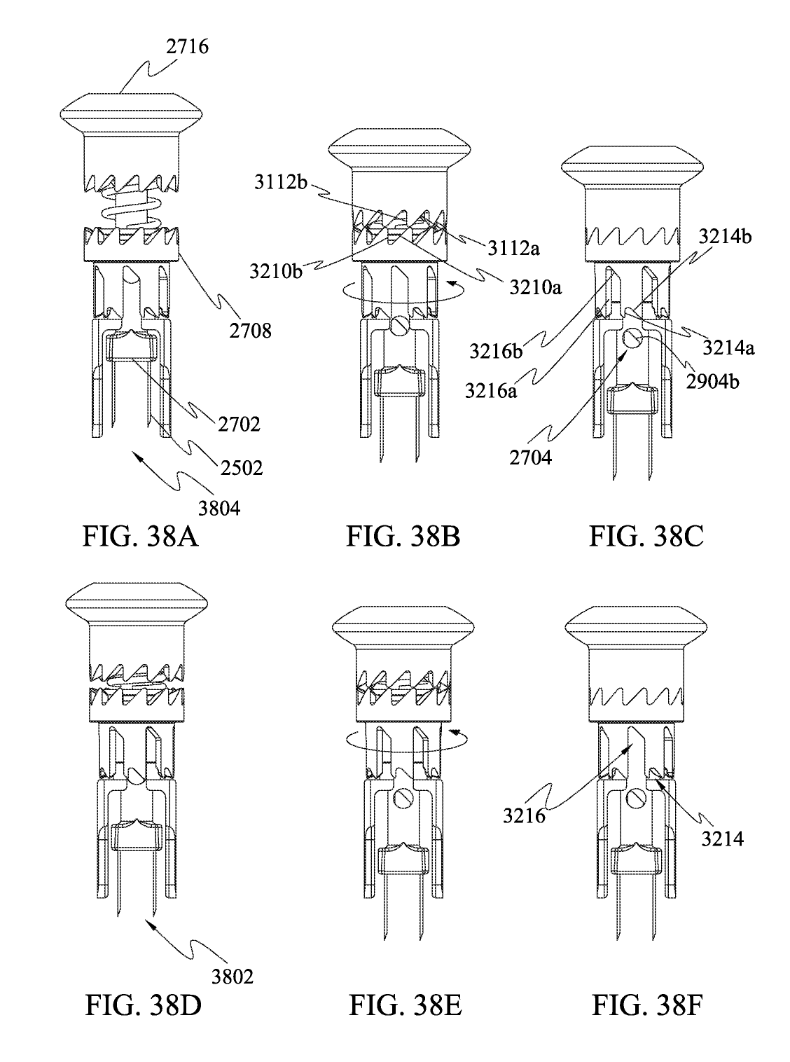

Referring to FIG. 38A-38F, the piercing members 2502 are illustrated in the extended position 3802 in FIG. 38D. In the extended position 3802, the engagement member 2704 is received by the extended position lock slot 3214. Referring to FIG. 38A, the piercing members 2502 are illustrated in the retracted position 3804. In the retracted position 3804, the engagement member 2704 is received by the retracted position lock slot 3216.

Pressing of the cap 2716 may push the shaft 2702 in a first direction, for example towards the skin 10, and may also enable partial rotation of the second engagement member 2708, such that the engagement member 2704 is aligned with one of a plurality of extended position lock slot 3214 defined in the second engagement member 2708. Releasing of the cap 2716 may enable the shaft 2702 to translate in a direction opposite to the direction in which it translated earlier. The translatory movement of the shaft 2702 will be stopped by engagement of the engagement member 2704 with the second engagement member 2708 at the extended position lock slot 3214, thereby enabling the piercing members 2502 to assume an extended position.

The piercing members 2502 may be made to retract out of the tissue from the extended position to a retracted position by pressing the cap 2716 again. Pressing of the cap 2716 pushes the shaft 2702 further, thereby disengaging the engagement member 2704 from the second engagement member 2708. Pressing of the cap 2716 may also enable partial rotation of the second engagement member 2708, such that the engagement member 2704 is now aligned with one of the retracted position lock slot 3216 defined in the second engagement member 2708. Releasing of the cap 2716 will enable the piercing members 2502, along with the shaft 2702 to translate out of the tissue. The translatory movement of the shaft 2702 may be stopped by engagement of the engagement member 2704 with the second engagement member 2708 at one of the plurality of retracted position lock slot 3216, thereby enabling the piercing members 2502 to assume the retracted position.

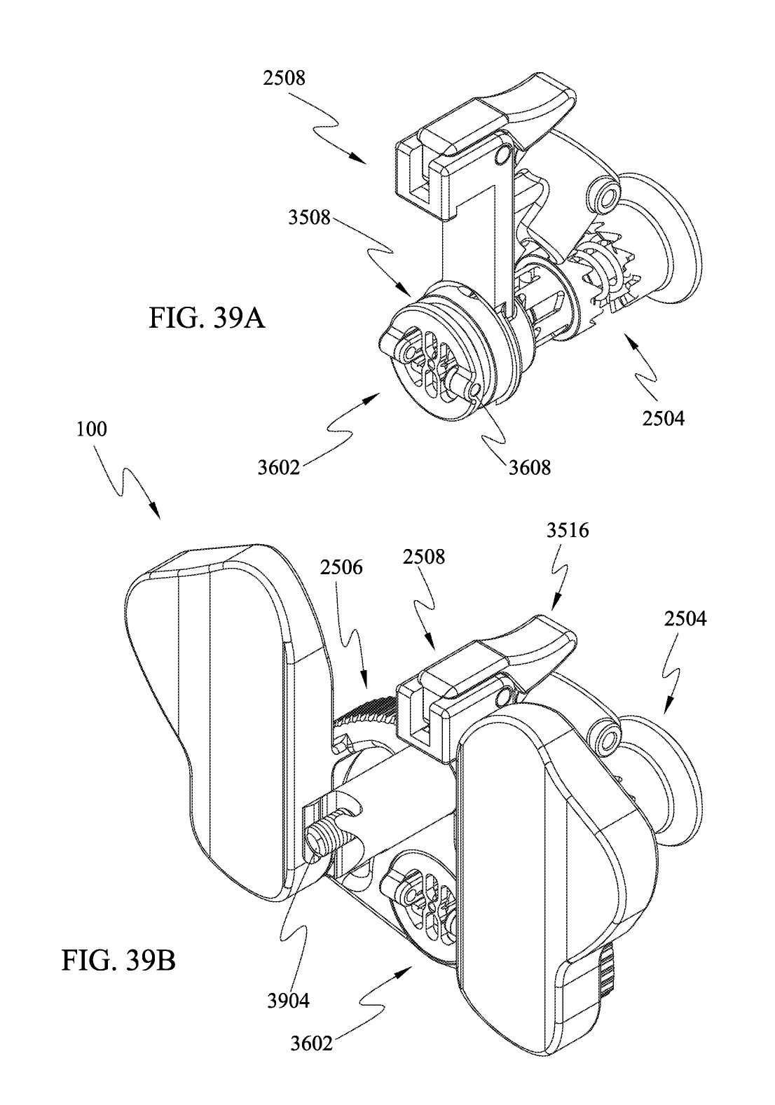

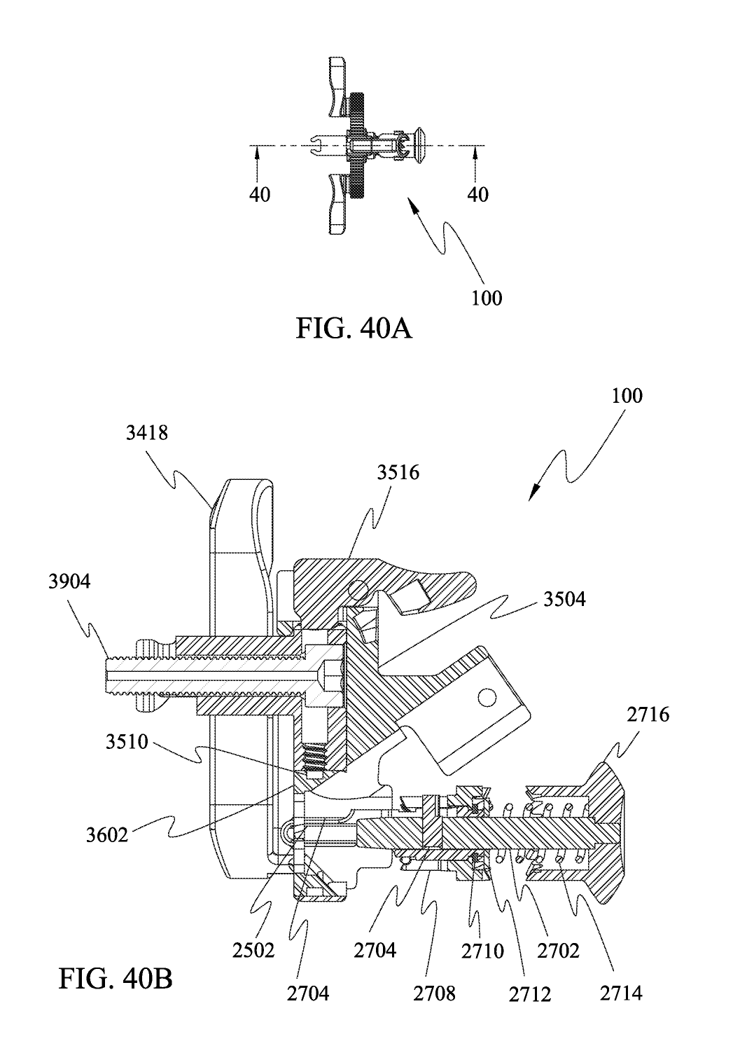

Referring to FIGS. 39A-40B, the assembly 2504 may be engaged with the arm 2508. Pivot holes 3608 defined in the counter pressure device 3602 may be aligned with the apertures 3326 defined in the pivot enabling member 2706. A pair of pivot pins may be engaged in the apertures 3326 and the pivot holes 3608, thereby enabling the assembly 2504 to pivot about a pivot axis.

The latch 3516 may be engaged to the arm 2508. The pivot pin 3530 (FIG. 35B) may be passed through the pivot hole 3528 defined in the latch 3516 and the pair of pivot openings 3514 defined in the extending member 3506, thereby enabling the latch 3516 to pivot.

The arm 2508 may be engaged to the guide member 2506. Engagement screws 3510 may be passed through the holes 3416 defined in the guide member 2506, such that a portion of the engagement screws 3510 extend into the groove 3508 defined in the arm 2508, thereby engaging the arm 2508 with the guide member 2506, while allowing the arm 2508 to rotate.

The guide member 2506 may be engaged with the system 110 which may be used to extract hair follicles from beneath the external surface of the skin 10. The additional aperture 3410 defined in the guide member 2506 may allow an engagement screw 3904 to engage the guide member 2506 with the system 110.

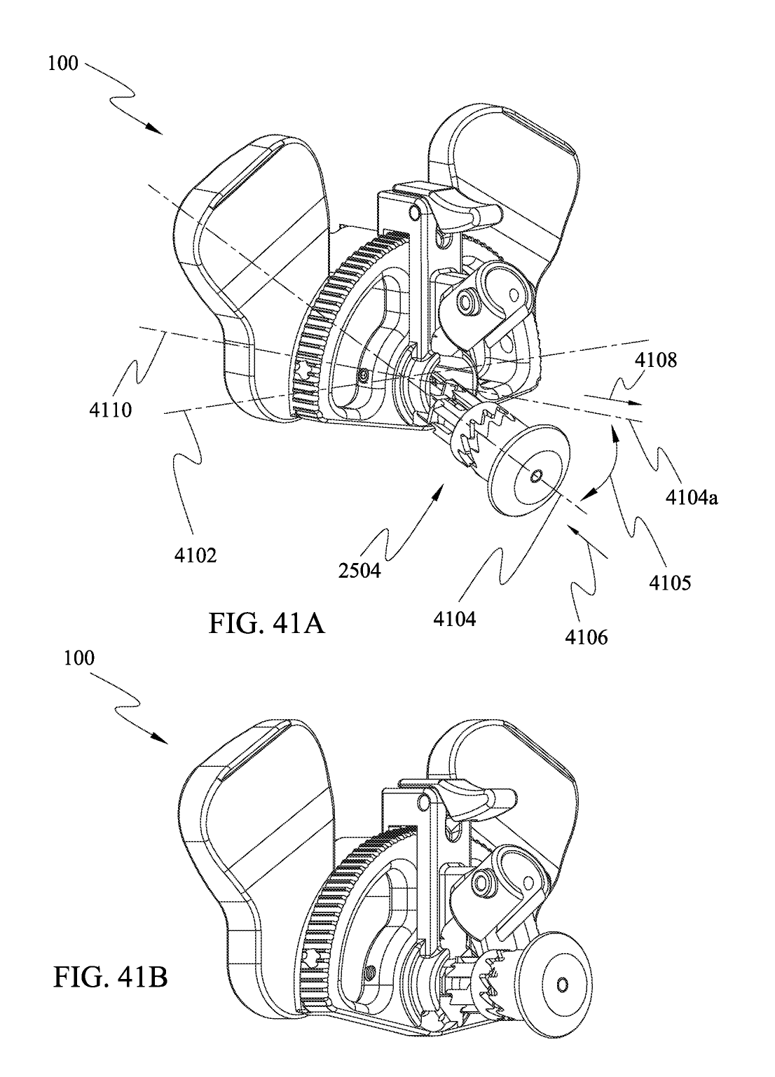

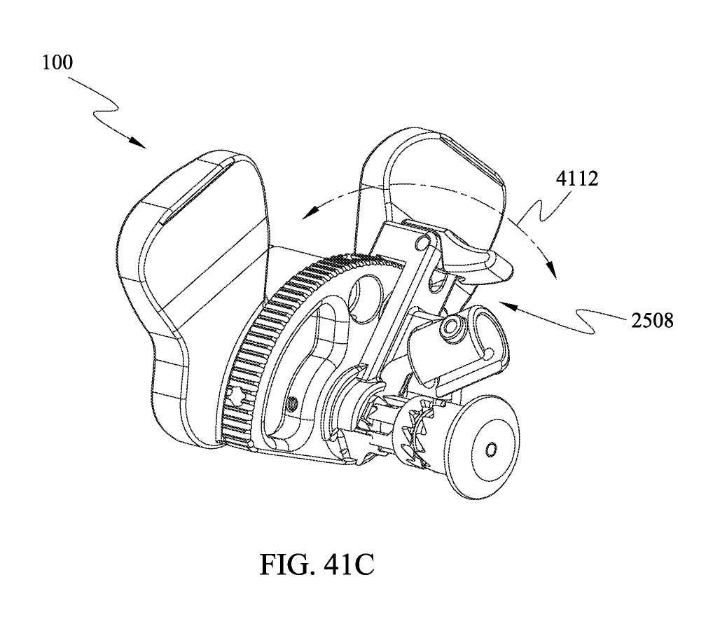

Referring to FIGS. 41A-41C, the system 100 may include a pivot axis 4102 such that the assembly 2504 may pivot about the pivot axis 4102. Pivoting the assembly 2504 about the pivot axis 4102 allows the piercing member 2502 to pivot about the pivot axis 4102.

The assembly 2504 may be pivoted as indicated by the arrow 4105 to choose a first chosen axis 4104. The piercing member 2502 may be configured to translate along the first chosen axis 4104 in a first direction 4106. The translation of the piercing member 2502 along the first chosen axis 4104 in the first direction 4106 may allow the piercing member 2502 to pierce through the external surface 11 of the skin 10. The first chosen axis 4104 may be chosen by pivoting the assembly 2504. The assembly 2504 may be pivoted so that the shaft 2702 or piercing members 2502 may be aligned with a second chosen axis 4104a. The piercing member 2502 may be configured to translate along the second chosen axis 4104a in a second direction 4108. The translation of the piercing member 2502 along the second chosen axis 4104a in the second direction 4108 may allow the piercing member 2502 to retract out of the skin. The pivot axis 4102 may intersect with the first and second chosen axis 4104, 4104a. The piercing member 2502 may be configured to translate along the first chosen axis 4104 in the first direction 4106 to assume an extended position 3802. The piercing member 2502 may be configured to translate along the second chosen axis 4104a in the second direction 4108 to assume a retracted position 3804.

The system 100 may further include a rotational axis 4110. The piercing member 2502 may be configured to rotate as indicated by arrow 4112 about the rotational axis 4110. The pivot axis 4102 may intersect the rotational axis. The chosen axis along which the piercing member 2502 retracts out of the skin may be the rotational axis 4110. The arm 2508 may be configured to rotate at least partially about the rotational axis 4110. The rotation of the arm 2508 about the rotational axis may allow the piercing members 2502 to rotate about the rotational axis. The rotational axis may intersect the pivot axis 4102. The arm 2508 may be configured to rotate about the rotational axis in either direction from a central position. The grooved engagement shapes 3420 may engage with the protruded portion 3524 provided at the tip of the beak shaped feature 3522 of the arm 2508 thereby allowing the arm 2508 to rotate about the engagement shapes 3420 and be locked at a desired position. The rotation of the piercing member 2502 may enable the piercing members 2502 to align with at least a hair follicle that may have a complex or compound angle.

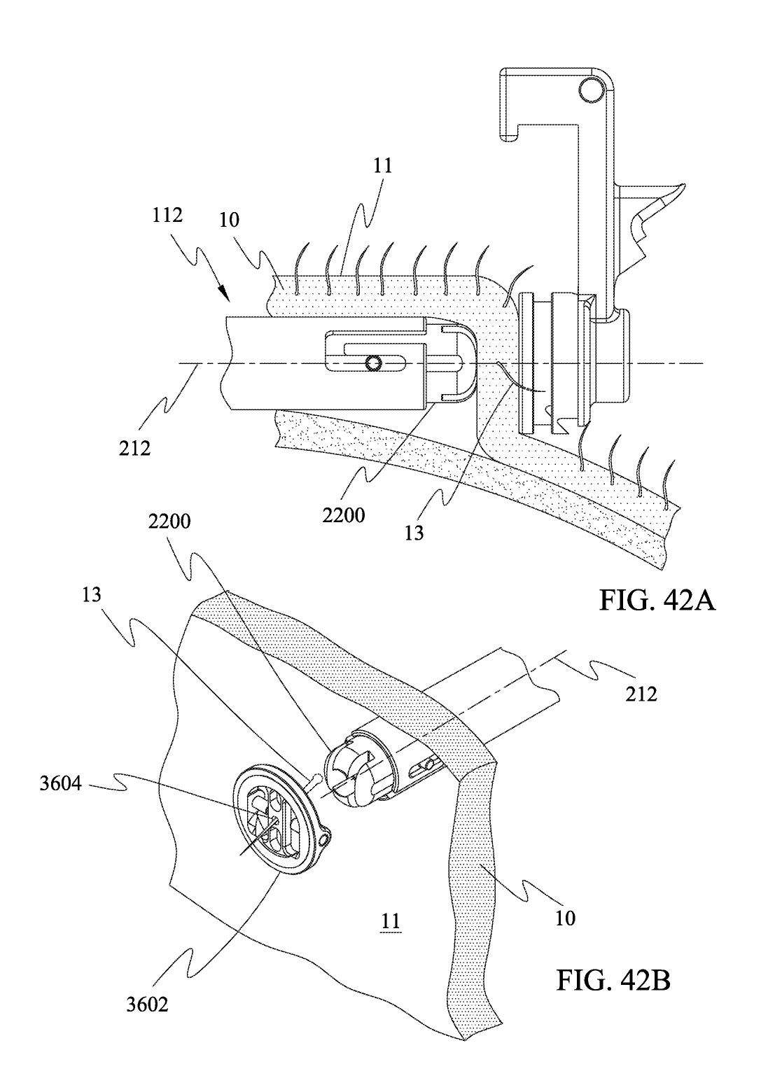

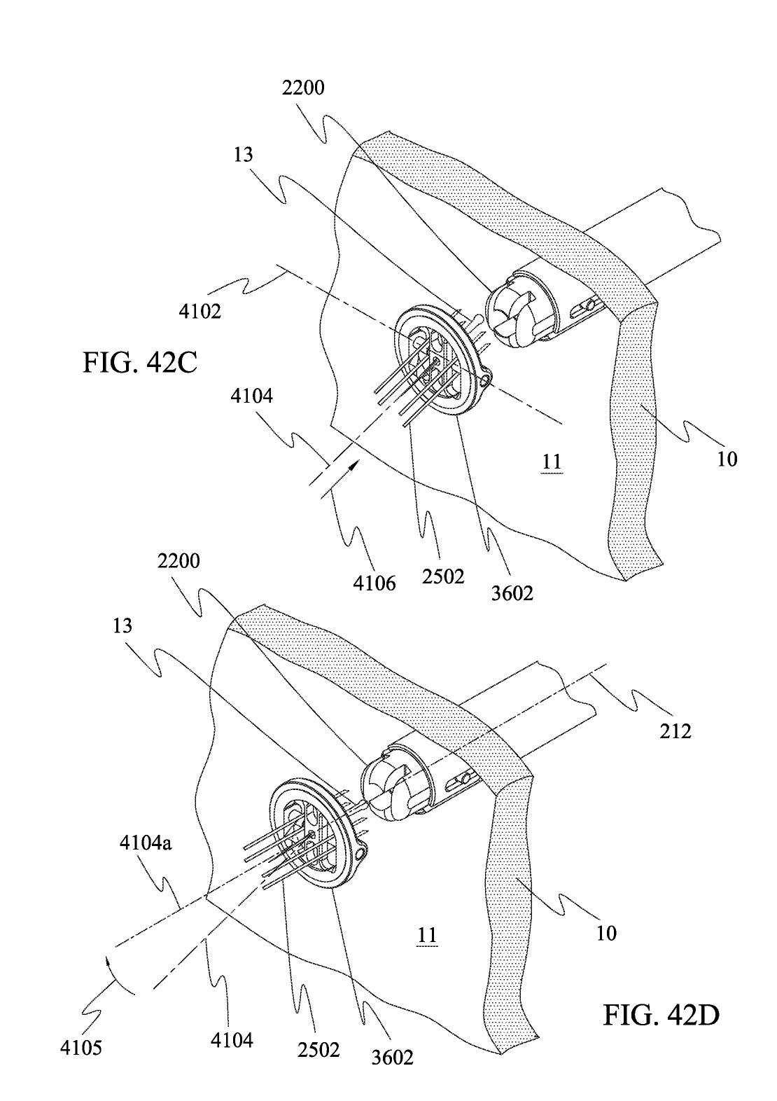

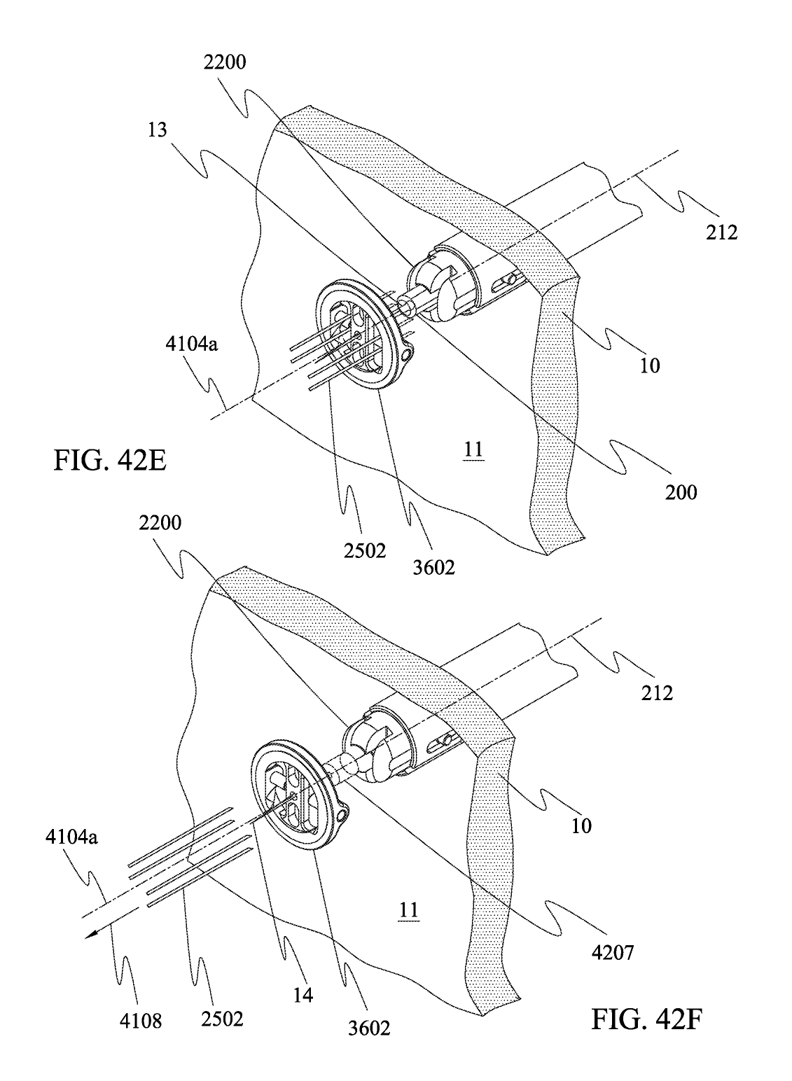

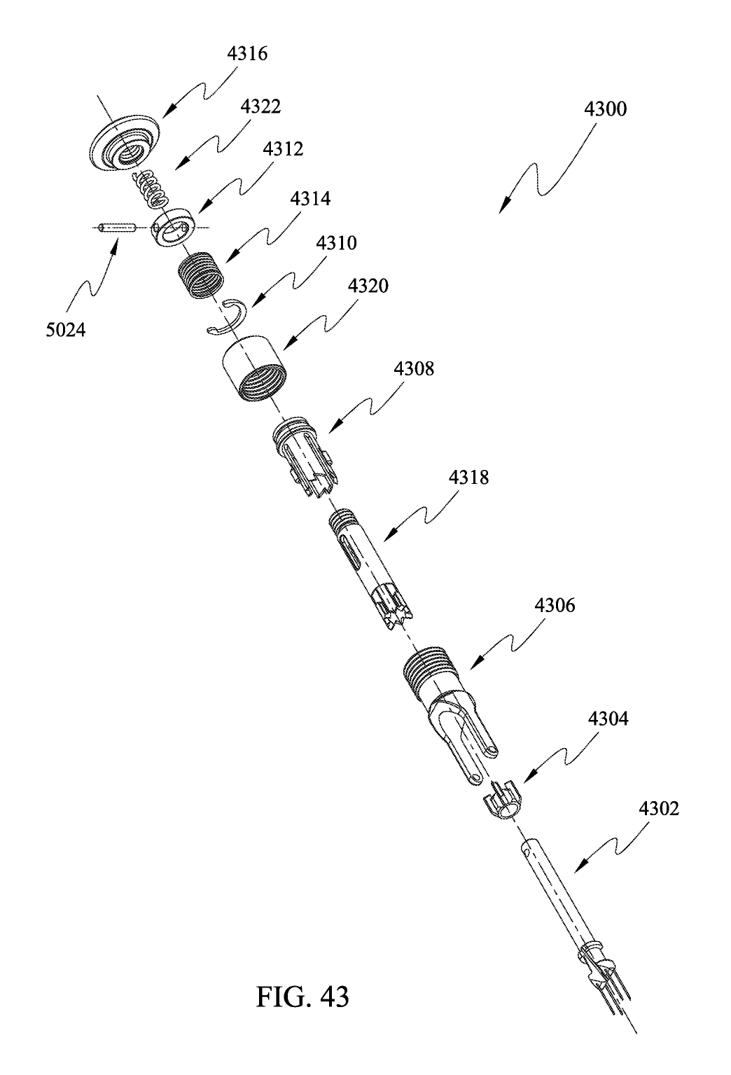

Referring to FIGS. 1C-1E and 42A-42F, the system 100 may be used in conjunction with the device 110. The device 110 may be employed to extract hair follicles from underneath the external surface 11 of the skin 10.

A portion 112 of the device 110 may be configured to enter an area beneath the external surface 11 of the skin 10 towards the donor region 114. An incision 120 may be made closer to the donor region 114. The incision 120 provides access to the portion 112 of the device 112 to be inserted underneath the skin 10, thereby providing access to hair follicles from underneath the external surface 11 of the skin 10.

The first member 200 may include a cutting tip towards its end. The first member 200 may be configured to rotate about a longitudinal axis 212 such that the rotation may enable the first member 200 to cut through a tissue that may have the target hair follicle. A second member may operate in conjunction with the first member 200 which may enable the device 110 to clip the hair follicles 13 which may have been cut by the first member 200.

The tissue stabilizing member 2200 may be configured to apply pressure against or around a tissue comprising a hair follicle 13 from underneath the external surface 11 of the skin 10. Once the tissue comprising the target hair follicle 13 may be stabilized and prepared for extraction, the first member 200 may translate along its longitudinal axis to cut the tissue by rotating about its longitudinal axis.

The guide member 2506 may be held against the external surface 11 of the skin 10. While holding the guide member 2506 against the external surface of the skin, the counter pressure device 3602 may apply pressure over or around the tissue comprising the hair follicle 13. The region around which the counter pressure device 3602 operates may be the external surface 11 of the region around which the tissue stabilizing member 2200 operates from underneath the skin 10. The tissue comprising the target hair follicle 13 may be disposed between the tissue stabilizing member 2200 and the counter pressure device 3602.

The hair follicle 13 to be extracted may be seen through the target aperture 3604 provided in the counter pressure device 3602. The hair follicle 13 may be misaligned with respect to the first member 200 or the tissue stabilizing member 2200. If the first member 200 were to cut through the tissue to extract the hair follicle 13 when it is misaligned, then the hair follicle might be transected, which is not desirable. Hence, aligning the hair follicle 13 with the axis of the first member 200 may be desired.

In order to align the hair follicle 13 with the first member 200, the piercing member 2502 may be rotated about the rotational axis depending on the alignment of the hair follicle 13. The piercing members 2502 may be pivoted about the pivot axis so that the axis of the piercing members 2502 may be parallel, or the axis of the shaft 2702 carrying the piercing members 2502 is in line, with the axis of the hair follicle 13.

After aligning the piercing members 2502, the piercing members 2502 may be translated into the skin in the first direction 4106 along the first chosen axis 4104. Thereafter, the piercing members 2502 may pivoted as indicated by the arrow 4105 about the pivot axis 4102 such that pivoting the piercing members 2502 may cause the hair follicle 13 disposed between the piercing members 2502 to pivot. Pivoting the piercing members 2502 may be required to align the hair follicle 13 disposed between the piercing members 2502 with the longitudinal axis 4208 of the first member 200 of the device 110. Once the axis of the hair follicle 13 is aligned with the longitudinal axis 4208 of the first member 200, the first member 200 of the device 110 may be operated to cut the hair follicle 13. The second member may be operated to clip the hair follicles 13 cut by the first member 200. The second member may clip and extract the portion underneath the external surface 11 of the skin 10 leaving a portion 14 of the hair follicle 13,14. The portion 14 may be the portion extending out of the external surface 11 of the skin 10. Once the extraction of the hair follicle 13 may be completed, the first member 200 and the second member may translate in a direction away from the external surface 4202, leaving behind a wound 4207 in the skin 10, without causing any wound on the external surface 4202. The piercing members 2502 may be retracted out of the skin 10 along the second chosen axis 4104a in the second direction 4108. The second chosen axis 4104a may be in line or parallel to the longitudinal axis of the first member 200.

Referring to FIG. 43, an assembly 4300 may include a shaft 4302, an engagement member 4304, a pivot enabling member 4306, a second engagement member 4308, a first ring 4310, a second ring 4312, a compression member 4314 and a cap 4316, an indexer 4318, a knob 4320 and a second compression member 4322.

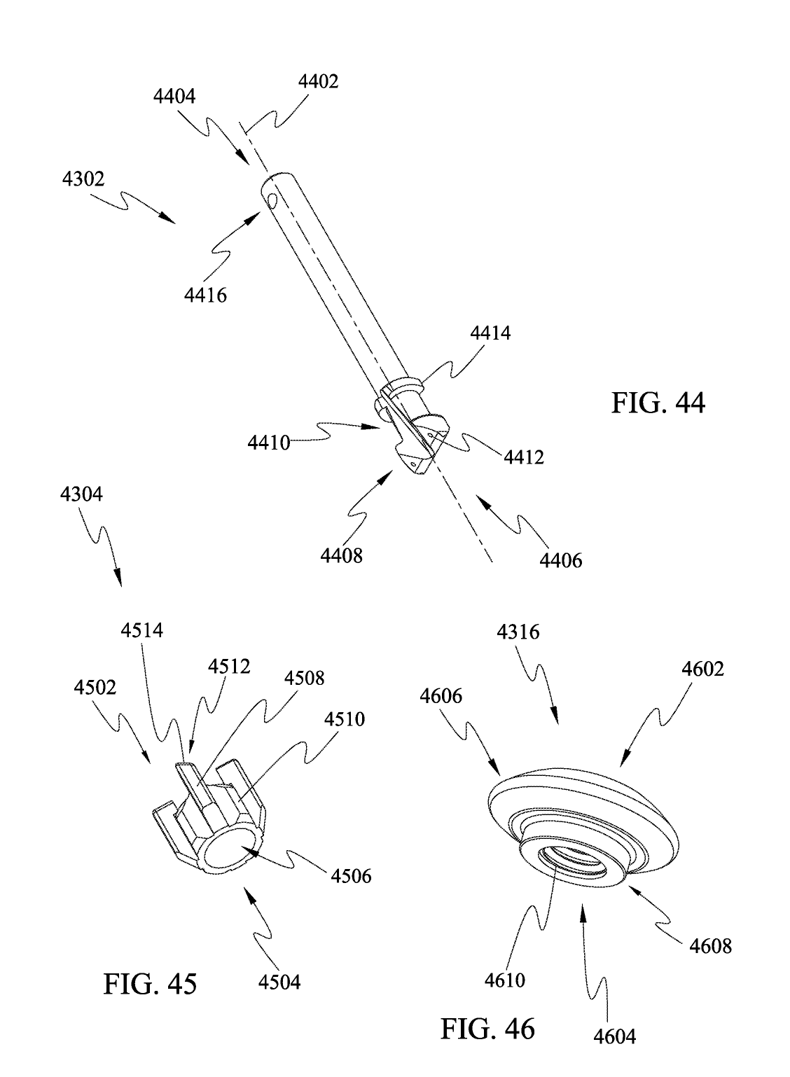

Referring to FIG. 44, the shaft 4302 may have portion that has a cylindrical cross section along its longitudinal axis 4402. The shaft 4302 may include a proximal end 4404 and a distal end 4406. The shaft 4302 may include a base 4408 at the distal end that may have a polygonal cross section. The base 4408 has a V shaped groove, which may enable a targeting system such as a microscope to capture image of hair extending out of a targeting aperture which may be provided in the counter pressure device 3602. The width of the base 4408 may be greater than the diameter or width of rest of the portion of the shaft 4302. One of the surfaces of the base 4408 may define a groove 4410.

The base 4408 of the shaft 4302 may include a plurality of first apertures 4412 extending from the distal end 4406 into the shaft 4302 parallel to the longitudinal axis 4402 of the shaft 4302. The first apertures 4412 may be configured to accommodate the piercing members 2502 such that a portion of the piercing members 2502 may be accommodated in the first apertures 4412, and a remaining portion which includes the pointed distal end 2604 protrudes out of the shaft 4302.

The shaft 4302 may define a shoulder 4414. The shoulder 4414 may have a circular cross section such that the shoulder 4414 may have a larger diameter than the diameter of a portion the shaft 4302, immediately succeeding the shoulder 4414 towards the proximal end 4404. The shaft 4302 may further define a through hole 4416 towards the proximal end 4404. The through hole 4416 may be perpendicular to the longitudinal axis 4402 of the shaft 4302.

Referring to FIG. 45, the engagement member 4304 may have a proximal end 4502 and a distal end 4504. The engagement member 4304 may define a through hole 4506 extending from the proximal end 4502 until the distal end 4504. The through hole 4506 may be configured to receive the shaft 4302, such that the engagement member 4304 rests over the shoulder 4414 of the shaft 4302.

The engagement member 4304 may define a plurality of laterally extending projection portions 4508 spread around the through hole 4506. An intermediate portion 4510 may be present between adjacent projection portions 4508. Outer diameter of the engagement member 4304 measured at the intermediate portions 4510 may be smaller than the outer diameter of the engagement member 4304 measured at the projection portions 4508. The outer diameter of the inner diameter of the second engagement member 4308 measured at the second engagement member arms 4808.

Each projection portion 4508 may define a tooth 4512 facing the proximal end 4502. The tooth 4512 may define an asymmetrical “V” or “U” shaped configuration. Each tooth 4512 may have a beveled surface 4514. The inclination of the beveled surface 4514 may be such that, force acting on the beveled surface 4514 may enable rotation of the engagement member 4304.

Referring to FIG. 46, the cap 4316 may be configured to be engaged with the indexer 4318. The cap 4316 may include a proximal end 4602 and a distal end 4604. The cap 4316 may include a lid portion 4606 and a cover portion 4608. The lid portion 4606 may be provided towards the proximal end 4602 of the cap 4316. The cover portion 4608 may extend from the lid portion 4606 towards the distal end 4604. The cap 4316 may define a threaded hole 4610 that may facilitate engagement of the indexer 4318 with the cap 4316.

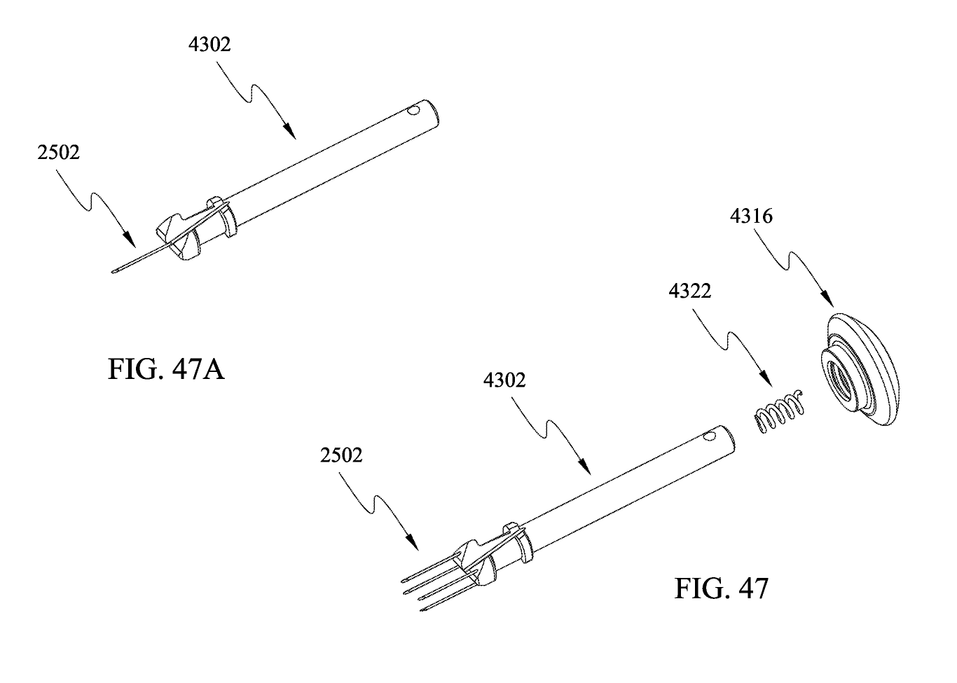

Referring to FIG. 47, the second compression member 4322 may be received by the indexer 4318, such that the second compression member 4322 may be disposed between the proximal end 4404 of the shaft 4302 and the lid 4606 of the cap 4316.

In FIG. 47, the shaft 4302 as illustrated is adapted with four piercing member 2502. The shaft 4302 may be alternatively adapted with a single piercing member 2502 (refer FIG. 47A). The single piercing member 2502 may be disposed along the longitudinal axis or the longitudinal center of the shaft 4302. Alternatively, the single piercing member 2502 may be offset from the longitudinal axis or the longitudinal center of the shaft 4302. In an embodiment, the shaft may be configured to be adapted with more than one piercing member.

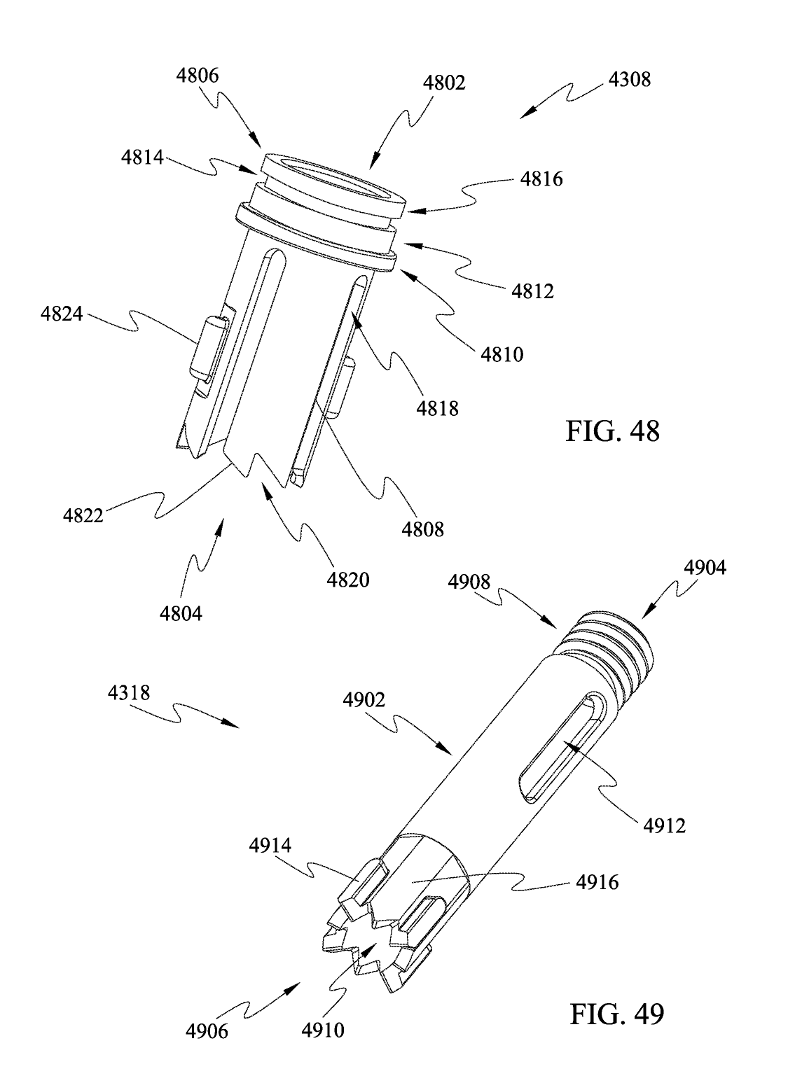

Referring to FIG. 48, the second engagement member 4308 may include a proximal end 4802 and a distal end 4804. The second engagement member 4308 may include a second engagement member head 4806 towards the proximal end 4802. The second engagement member 4308 may include a plurality of second engagement member arms 4808. The second engagement member arms 4808 may extend from the head 4806 towards the distal end 4804 of the second engagement member 4308.

The second engagement member head 4806 may include a shoulder 4810, a first neck 4812, a second neck 4814 and a second head 4816. The shoulder 4810 may have an outside diameter that may be greater than the outside diameter of adjoining portion of the second engagement member 4308 towards the distal end 4804. The first neck 4812 may have an outside diameter that may be smaller than the outside diameter of the shoulder 4810. The second neck 4814 may have an outside diameter that may be smaller than the outside diameter of the first neck 4812. The second head 4816 may have an outside diameter that may be larger than the outside diameter of the second neck 4814.

The second engagement member arms 4808 may extend in a second direction towards the distal end 4804 from the shoulder 4810. A through slot or retracted position lock slot 4818 may be defined between adjacent second engagement member arms 4808.

Each second engagement member arms 4808 may define an extended position lock slot 4820. Each of the extended position lock slot 4820 may define a shape that may be complimentary to the shape of the tooth 4512 defined by projection portion 4508 of the engagement member 4304.

Each second engagement member arms 4808 may define rotation enabling surface 4822 adjacent to the extended position lock slot 4820. The rotation enabling surface 4822 may be a beveled surface configured such that a member pressed against the rotation enabling surface 4822 may slide into the retracted position lock slot 4818.

The outer surface of two diametrically opposing second engagement member arms 4808 may define protruded features 4824. The protruded features 4824 may have the cross section of a half cylinder. The protruded features 4824 may be configured such that they engage with complimentary feature provided in the pivot enabling member 4306.

Referring to FIG. 49, the indexer 4318 may have a cylindrical body 4902 comprising a proximal end 4904 and a distal end 4906. The proximal end of the cylindrical body 4902 may include a neck 4908, which may be threaded. The neck 4908 may be configured to be received by the threaded portion 4610 of the cap 4316.

The body 4902 of the indexer 4318 may define a hole 4910 which may be configured to receive the shaft 4302. While receiving the shaft 4302, the second compression member 4322 may be received inside the threaded neck 4908 of the indexer 4318. The body 4902 of the indexer 4318 may define through slots 4912 that may be diametrically opposing.

The body 4902 of the indexer 4318 may define a plurality of first indexing teeth 4914 and second indexing tooth 4916. Each of the first indexing teeth 4914 may be present between adjacent second indexing teeth 4916. The outer diameter of the indexer 4318 measured at the first indexing tooth 4914 may be larger than the outer diameter of the indexer 4318 measured at the second indexing tooth 4916. Each of the first indexing teeth 4914 may be in the form of a protrusion.

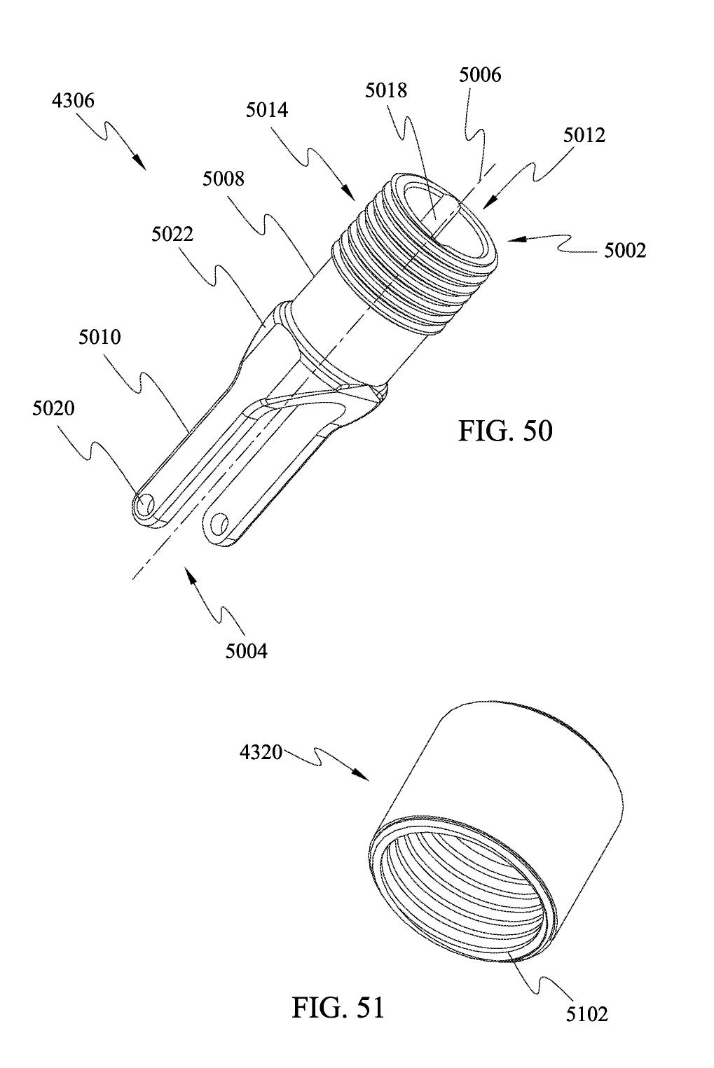

Referring to FIG. 50, the pivot enabling member 4306 may have a proximal end 5002, a distal end 5004 and a longitudinal axis 5006. The pivot enabling member 4306 may include a pivot enabling member head 5008 and a pair of pivot enabling member arms 5010. The head 5008 may define a through hole 5012 configured to allow the shaft 4302 to translate along the axis 5006. A portion of the pivot enabling member head 5008 may include threads 5014 that may engage with complimentary threads 5102 provided in the knob 4320. The pivot enabling member 4306 may define protruded feature receiving slots 5018 in the inner surface of the pivot enabling member head 5008. The protruded feature receiving slots 5018 may be configured to receive protruded features 4824 defined in the second engagement member arms 4808.

The pivot enabling member arms 5010 extend from the pivot enabling member head 5008 towards the distal end 5004. Each of the arms 5010 may define apertures 5020 configured to engage with pivot pins, thereby enabling the pivot enabling member 4306 to pivot.

Each of the arms 5010 may be define a beveled surface 5022 configured to mate and fit with the beveled surface 3501 provided in the cylindrical body 3502 of the arm 2508.

The first ring 4310, which may be a retaining ring, may be engaged with the second neck 4814 of the second engagement member 4308. The second ring 4312 may be disposed on top of the compression member 4314. The second ring 4312 may define a through hole 5022 which may align with the through hole 4416 defined in the shaft 4302. The second ring 4312 and the shaft 4302 may be engaged by means of a pin 5024 that may be passed through the through hole 5022 defined in the second ring 4312 and the through holes 4416 defined in the shaft 4302.

Referring to FIG. 51, the knob 4320 may be configured to engage with the pivot enabling member 4306. The inner surface of the knob 4320 may have threads 5102 to enable engagement with the pivot enabling member 4306. The knob 4320 may be configured such that it encircles the shaft 4302 and is positioned between the proximal end 4404 and distal end 4406 of the shaft 4302.

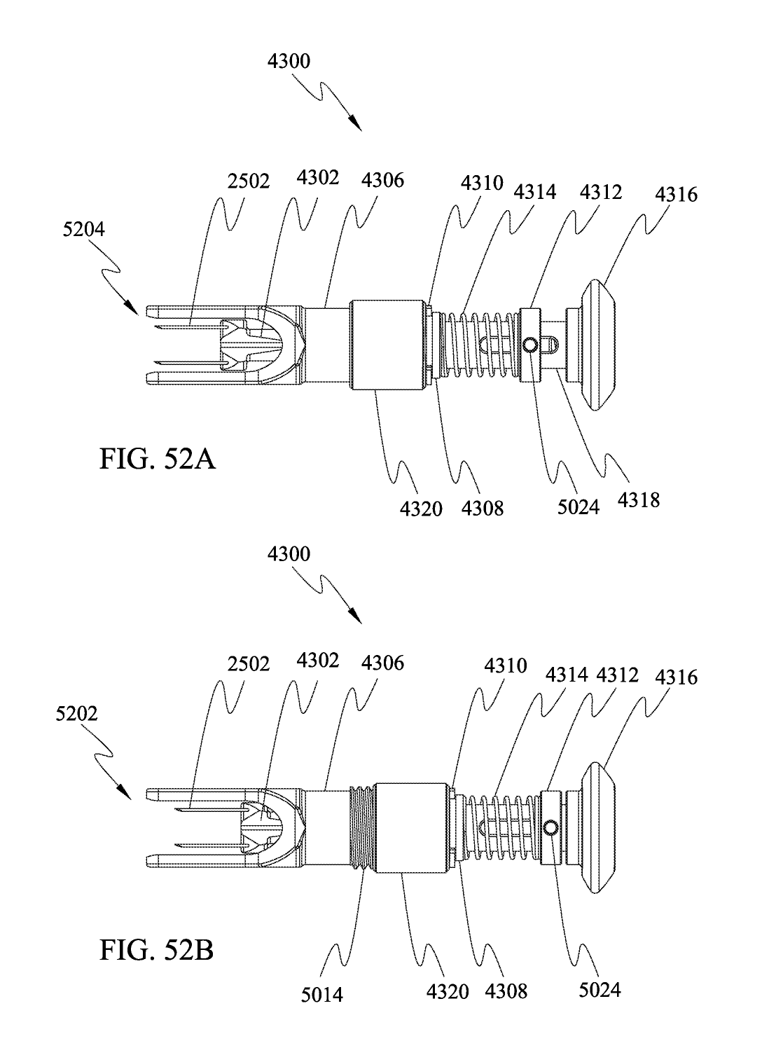

Referring to FIGS. 52A-52B, the knob 4320 may be operable to translate the shaft 4302 along a longitudinal axis of the shaft to configure the extent to which the piercing member 2502 is extended into the external surface of the skin when the piercing member 2502 is locked in the extended position. The engagement of the pivot enabling member 4306, second engagement member 4308 and the shaft 4302 with the knob 4320 may be such that the rotation of the knob 4320 may enable the shaft 4302 to partially translate in a first direction towards the external surface of the skin. Similarly the rotation of the knob 4320 in a direction opposite to the earlier direction may enable the shaft 4302 to partially translate in a second direction to move away from the external surface of the skin.

The knob 4320 may be rotated in a first direction. The rotation of the knob 4320 in the first direction may enable the knob 4320 to translate in a direction towards the cap 4316. The rotation of the knob 4320 in the first direction and the translatory motion of the knob 4320 in the may cause the compression member 4314 disposed over the knob 4320 to translate towards the cap 4316. The motion of the compression member 4314 may push the second ring 4312 towards the cap 4316. The second ring 4312, being engaged with the shaft 4302 through the pin 5024 may cause the shaft 4302 to move closer to the cap 4316, thereby decreasing the length, as indicated by arrow 5204, of the piercing members 2502 that would be extended out of the assembly 4300 or into the skin, when the assembly 4300 is operated to lock the piercing members 2502 in the extended position.

In order to increase the length, as indicated by arrow 5202, of the piercing members 2502 that would be extended out of the assembly 4300 or into the skin, when the assembly 4300 is operated to lock the piercing members 2502 in the extended position, the knob 4320 may be rotated in a second direction, opposite to the first direction. Rotation in the second direction may cause the knob 4320 to translate away from the cap 4316, allowing the second compression member 4322 to act on the shaft 4302, and push the shaft 4302 away from the cap 4316.

Referring to FIGS. 53A-53J, the shaft 4302 may be received through the engagement member 4304 such that the engagement member 4304 rests on top of the shoulder 4414. A portion of the shaft 4302 may be received by the indexer 4318 such that the through hole 4416 defined in the shaft 4302 and the through slot 4912 defined in the indexer 4318 may be aligned. The second engagement member 4308 may be received such that it encircles a portion of the indexer 4318. The pivot enabling member 4306 may be engaged with the second engagement member 4308, such that the pivot enabling member head 5008 may be pressed against the bottom of the shoulder 4810 of the second engagement member 4308. The first ring 4310 may be engaged around the second neck 4814 of the second engagement member 4308. The second ring 4312 may be disposed on top of the compression member 4314. The compression member 4314 may be placed below the second ring 4312 and on top of the head 22816 of the second engagement member 4308. The knob 4320 may be engaged around the pivot enabling member 4306. The neck 4908 of the indexer 4318 may be received by the threaded hole 4610 defined in the cap 4316. The neck 4908 of the indexer 4318 may be engaged with the cover portion 4608 and the lid portion 4606 of the cap 4316. The proximal end 4404 of the shaft 4302 may be received through the pivot enabling member 4306, the second engagement member 4308, the indexer 4318, the first ring 4310, the second ring 4312, and the compression member 4314. The second compression member 4322 may be disposed between the proximal end 4404 of the shaft 4302 and the lid portion 4606 of the cap 4316 and may be received by the indexer 4318.

Referring to FIGS. 54A-54H, the piercing members 2502 may be made to pierce through an external surface of the skin, by pressing the cap 4316. Pressing of the cap 4316 may push the shaft 4302 towards the skin.

In the retracted position, the projection portions 4508 may be received in the retracted position lock slot 4818. Upon pressing the cap 4316, the first indexing tooth 4914 defined in the indexer 4318 may push the projection portions 4508 defined in the engagement member 4304. The engagement member 4304 moves downwards along the retracted position lock slot 4818. Once the projection portions 4508 moves out of the retracted position lock slot 4818, the projection portions 4508 begins to slide against the slanted surface of the first indexing tooth 4914, causing the engagement member 4304 to rotate. The engagement member 4304 which is being forced to move towards the cap 4316 by the compression member 4314 may begin to slide into the extended position lock slot 4820 and engage in the extended position lock slot 4820 to assume the extended position.

The retracted position may be assumed from the extended position by pressing the cap 4314. Pressing the cap 4316 may cause the second indexing tooth 4916 to push the projection portions 4508 thereby causing a partial rotation of the engagement member 4304. The engagement member 4304 may continue to rotate until it aligns with the rotation enabling surface 4822. The rotation enabling surface 4822 may further allow the engagement member 4304 to rotate further until the projection portions 4508 slides into the retracted position lock slot 4818. The engagement of the projection portions 4508 into the retracted position lock slot 4818 may stop further rotation of the engagement member 4304. The engagement member 4304 may now translate in the slot 4818 towards the cap 4314 to assume the retracted position.

Referring to FIG. 55, a system 5500 may be provided to collect hair follicles extracted from underneath the external surface of the skin using the system 110. The system 5500 may include a fluid reservoir 5502, a pump 5504, a controller 5506, the system 110, a collection assembly 5508 and a suction mechanism 5510.

The fluid reservoir 5502 may be connected to the pump 5504 by a tube. A second tube may be used to connect the pump 5504 to the system 110. The fluid reservoir 5502 may serve as a source for supplying fluid to the system 110. The fluid present in the fluid reservoir may be saline. The fluid from the fluid reservoir 5502 may be pumped into the system 110 via the pump 5504. The pump 5504 may be connected to the controller 5506 such that the flow of the fluid into the system 110 may be controlled. The controller 5506 may include a foot paddle configured to turn on, turn off or control the flow of fluid from the pump 5504 to the system 110.

The pump may be used if the fluid reservoir 5502 is at a lower altitude compared to the altitude of the system 110. Alternatively, the fluid may be gravity fed to the system 110 from the fluid reservoir 5502.

The system 110 may include the inlet port 2438, cover member 2100, first member 200, chamber 616 and an outlet port 618. In FIG. 55, the parts of the system 110 enclosed within the dotted box may be configured to at least partially translate or be moved underneath the external surface of the skin. The fluid from the fluid reservoir 5502 may be received into the system 110 through the inlet port 2348. The inlet port 2348 may be engaged to the cover member 2100. The cover member 2100 may receive the fluid entering through the inlet port.

The cover member 2100 may be configured to enclose the first member 200. The first member 200 may include a proximal end and a distal end. The distal end may define a cutting edge, configured to cut through tissue having a hair follicle. The fluid flowing through the cover member 2100 may move towards the distal end of the first member 200, and enter the first member 200 at the distal end. Thereafter, the fluid may flow from the distal end towards the proximal end of the first member 200 because of negative pressure or suction.

The negative pressure may be created by having the proximal end of the first member 200 in fluidic communication with the chamber 616. The chamber 616 may be in fluidic communication with the outlet port 618. At the outlet port 618 negative pressure may be created or suction is applied by the suction mechanism 5510.

The flow of fluid towards the proximal end of the first member 200 may enable collection of the hair follicle, as the tissue comprising the hair follicle may be disposed within the first member 200 once the hair follicle has been cut or cored and clipped from underneath the surface of the skin using the cutting edge.

The extracted hair follicle may flow into the chamber 616, and then exit through the outlet port 618. The fluid having the hair follicles exiting the outlet port 618 may be collected in collection assembly 28. The collection assembly 5508 may adopt various mechanisms to collect the hair follicles present in the fluid. The collection assembly 5508 may be intermediate to the outlet port 618 and the suction mechanism 5510.

Referring to FIGS. 56A-56B, the system 110 may define a first fluid pathway 5600. The pathway defined by the system 110 for flow of fluid between the inlet port 2438 and the outlet port 618 may be referred to as the first fluid pathway 5600. The fluid enters the system 110 through the inlet port 2438. The fluid from the inlet port 2438 enters the aperture 2120 defined in the cover member 2100. The aperture 2120 as explained earlier may be in fluidic communication with the channel 2118 defined in the cover member 2100. Hence, the fluid entering the aperture 2120 may flow through the channel 2100 towards the distal end of the cover member 2100. The fluid may also pass through a channel defined in the bushing 2436, which is received by the cover member 2100. Subsequently, the fluid may enter the first member 200 through one or more openings, such as the opening of the first bore 206 or the apertures 222, provided towards its distal end. The entry of the fluid into the first member 200 or first bore 206 may be enabled by negative pressure or suction established at the outlet port 618, which may be in fluidic communication with the chamber 616, which in turn may be in fluidic communication with the first bore 206. The fluid entering the first bore 206 towards its distal end may now travel towards the proximal end of the first bore 206. The fluid may carry tissue that may have been clipped or cut or cored and disposed in the first bore 206. The fluid may exit the first bore 206 and enter the adapter 400. The fluid may flow through the bore 420 defined in the adapter 400. The fluid may exit the adapter 400 through the aperture 418 or exit port 418 defined in the adapter 400. The fluid exiting the exit port 418 may enter the chamber 616, which may encompasses or encircle or enclose the exit port 418. The fluid may exit the chamber 616 from the outlet port 618. The fluid exiting the outlet port may be received by the collection assembly 28.

The channel 2118 defined in the cover member 2100 may be referred to as a first portion of the first fluid pathway 5600. The channel 2118 or the cover member 2100 may be stationary. In other words, channel 2118 or the cover member 2100 may not rotate or translate or traverse along an axis, while it may be configured to be moved underneath the skin. The chamber 616 may be referred to as a second portion of the first fluid pathway 5600. The chamber 616 may translate along an axis, while being rotationally stationary. The first bore 206 and the bore 420 defined in the adapter 400 may be referred to as a third portion of the first fluid pathway 5600. The first bore 206 and the bore 420 may be configured to rotate and also translate along a axis, for example rotate about and translate along the longitudinal axis of the first bore 206.

The circulation of the fluid in the channel 2118 and the first bore 206 may define a generally “U” shaped pathway, in which channel 2118 may form a first leg of the “U” shaped pathway, and the first bore 206 may form a second leg of the “U” shaped pathway. The direction of flow of fluid in the first bore 206 may be opposite to the direction of flow of fluid in the channel 2118. The channel 2118 may be parallel to the first bore 206.

The fluid exiting the outlet port 618 may be received by the collection assembly. Referring to FIG. 57, a collection assembly 5700 having a semi permeable member 5710 may be provided. The collection assembly 5700 may include a cap 5702, an inlet 5704, a container 5706 and an outlet 5708. The collection assembly 5700 may receive the fluid having the hair follicle through the inlet 5704. The cap 5702 may include the inlet 5704 to facilitate the entry of fluid having the hair follicles into the container 5706. The cap 5702 may include an inlet 5704 by way of an aperture or through hole defined in the cap 5702. The container 5706 may have a proximal end and a distal end. The proximal end of the container 5706 may be covered by a cap 5702. The outlet 5708 may be provided at the distal end of the container 5706. An aperture or through hole at the distal end of the container 5706 may define the outlet 5708.

The semi permeable member 5710 may be placed within the container 5706. The semi permeable member 5710 may be placed within the container 5706 in such a way that it facilitates the exit of fluid through the semi permeable member 5710 towards one end of the semi permeable member 5710 facing the outlet 5708, while the hair follicles or grafts may be retained at the other end of the semi permeable member 5710 facing the inlet 5704. The collection assembly 5700 may be an example of a collection assembly adopting filtration mechanism for collecting the tissues or hair follicles or grafts.

The outlet 5708 may be configured with the suction mechanism. On application of suction using the suction mechanism, the fluid having the graft flows from the outlet port 618 provided in the system 110 into the collection assembly 5700. The collection assembly 5700 collects the hair follicles, while the fluid exits the collection assembly 5700 from the outlet 5708.

A cap may be provided at the distal end as well. The semi permeable member 5710 may be extracted out of the container 5706, and the hair follicles that are settled on the semi permeable member 5710 may be collected.

Referring to FIG. 58, a collection assembly 5800 with multiple collection chambers 5802 may be provided. The collection assembly 5800 may include an inlet 5804, an outlet 5806, tubes 5808, a plurality of collection chambers 5802 and a plurality of valves 5810.

The tubes 5808 receive fluid along with the hair follicles through the inlet 5804. The tubes 5808 may be configured to have a first pathway 5812 and a second pathway 5814.

Each pathway 5812, 5814 may include a collection chamber 2702. Further, each pathway 5812, 5814 may include plurality of valves 5810, and the collection chamber 5802 in each pathway 5812, 5814 may be placed intermediate the valves 5810. The collection chambers 5812 may include a filtering mechanism to separate hair follicle from the fluid. The filtration mechanism may be similar to the one described in connection with collection assembly 5700. The valves 5810 configured in a pathway, for example the second pathway 5814, may be closed to restrict flow of fluid along the second pathway 5814. The pathways 5812, 5814 may converge towards the outlet 5806.

Referring to FIGS. 59A-59C, a collection assembly 5900 adopting a filtration mechanism for collecting the hair follicles may be provided. The collection assembly 5900 may include an inlet 5902, a cap 5904, a filtration device 5906, a container 5908 and an outlet 5910. The cap 5904 may include the inlet 5902 to facilitate the entry of fluid having the hair follicles into the filtration device 5906. Upon filtration, the fluid passes from the filtration device 5906 into the container 5908 of the collection assembly 5900, while the hair follicles may be collected in the filtration device 5906. The outlet 5910 may facilitate the exit of fluid from the container 5908.

The cap 5904 may include an inlet 5902 by way of an aperture or through hole defined by the cap 5904. The configuration of a bottom surface of the cap 5904 may be such that the filtration device 5906 may get engaged to the bottom surface of the cap 5904.

The filtration device 5906 may include a proximal end and a distal end. The proximal end of the filtration device 5906 may be configured to be engaged to the bottom surface of the cap 5904. The proximal end of the filtration device 5906 may be engaged to the bottom surface of the cover cap 5904 by thread mechanism or key and slot mechanism, among other mechanisms.

The filtration device 5906 may be positioned in such a manner that the inlet 5902 of the collection assembly 5900 may be within the circumference of the filtration device 5906, so that the fluid exiting the inlet 5902 is led into the filtration device 5906. The filtration device 5906 may include mesh like structure 5912 and a collection bed 5914. The mesh like structure 5912 may be present on the lateral side of the filtration device 5906 and on the collection bed 5914. The mesh like structure may facilitate the exit of the fluid from the filtration device 5906, while arresting the hair follicles within the filtration device 5906. The hair follicles arrested in the filtration device 5906 may be collected or may rest on the collection bed 5914 at the distal end of the filtration device 5906. The filtration device 5906 may be detached from the cap 5904 and the hair follicles may be collected from the filtration device 5906.

The container 5908 of the collection assembly 5900 may be configured to receive the fluid exiting the filtration device 5906. The container 5908 may include a proximal end and a distal end. The proximal end of the container 5908 may be covered by the cap 5904. The filtration device 5906 may be intermediate to the cap 5904 and an inner surface of the container 5908 such that a gap may be defined between the collection bed 5914 of the filtration device 5906 and the distal end of container 5908. The proximal end of the container 5908 may include the outlet 5910. The fluid collected in the container 5908 may exit through the outlet 5910. The outlet 5910 may be provided towards the proximal end of the container 5908 and over the collection bed 5914 such that the hair follicles collected in the collection bed 5914 of the filtration device 5906 may always be in contact with the fluid or may be immersed in fluid, which may enhance the usability of the hair follicles.

Referring to FIGS. 60A-60C, a collection assembly 6000 having a plurality of baffles to separate the hair follicles from the fluid may be provided. The collection assembly 6000 may adopt weight based separation mechanism for collecting the tissue. The collection assembly 6000 may include an inlet 6002, an outlet 6004, an exit 6006, a container 6008, a cap 6010 and a baffles assembly 6012.

The container 6008 may have an open top. The top of the container 6008 may be closed by the cap 6010. The container 6008 may include side walls and bottom wall. Inside surface of two or more side walls may define a stepped configuration 6014. The inside surface or collection bed 6016 of the bottom wall may be inclined. The inclination may be such that fluid or hair follicles flow towards the exit 6006, which may be defined in the bottom wall or in the side wall at a portion closer to the bottom wall. One of the side walls may define the outlet 6004. The outlet 6004 may be provided at a position that may be intermediate the inlet 6002 and the exit 6006.

The baffles assembly 6012 may include a rim 6018. A plurality of baffles 6020 may be arranged along the rim 6018. The baffles 6020 may be arranged such that empty space may be defined between adjacent baffles 6020. The baffles 6020 may have varying height. The baffle 6020 having the shortest height may be positioned at one extreme of the baffles assembly 6012 and the baffle 6020 having the longest height may be positioned at the other extreme of the baffles assembly 6012.

The baffles assembly 6012 may be assembled with the container 6008. The rim 6018 may be positioned over the stepped configuration 6014 defined in the container 6008. The baffles assembly 6012 may be positioned such that the baffle 6020 having the longest height may be closer to the exit 6006.

During operation, the fluid having hair follicles may enter the container 6008 through the inlet 6002. The hair follicles, which may be heavier than the fluid may tend to move towards the collection bed 6016. As the hair follicles move towards the outlet 6004, it may interface with one of the baffles 6020, which may ensure that the hair follicles moves and settles on the collection bed 6016, instead of exiting from the outlet 6004. The hair follicles may slide towards the exit 6006 and may be eventually taken out of the container 6008 from the exit 6006.

Referring to FIGS. 61A-61B, a collection assembly 6100 may adopt hydro cyclone or centrifugal separation mechanism for collecting the tissue. The collection assembly 6100 includes an inlet 6102, an outlet or overflow 6104, an exit 6106, a tissue reception container 6108 and a cyclone body 6110 having a conical section 6112.

The inlet 6102 may be disposed tangentially with respect to the cyclone body 6110, such that fluid is received tangentially into the cyclone body 6110 from the inlet 6102. The cyclone body may be in fluidic communication with the outlet 6104 towards its superior end. The conical section 6112 may extend towards the inferior side of the cyclone body 6110. The conical section 6112 may be in fluidic communication with the tissue reception container 6108 at its inferior end. The tissue reception container 6108 may be in fluidic communication with the exit.

Fluid along with tissue may be received into the collection assembly 6100 from the inlet 6102 tangentially into the cyclone body 6110. A cyclone may be created in the cyclone body 6110. The tissue, which may be heavier compared to the fluid may advance towards the inferior end of the cyclone body 6110, and eventually exit the cyclone body 6110. The tissue exiting the cyclone body 6110 may be received by the tissue reception container 6108. The tissue received by the tissue reception container 6108 may exit the collection assembly from the exit 6106. The fluid or most of the fluid may advance towards the superior side of the cyclone body 6110 and exit the collection assembly 6100 from the outlet 6104.

Referring to FIGS. 62A-62C, a collection assembly 6200 may adopt hydro cyclone or centrifugal separation mechanism for collecting the tissue. The collection assembly 6200 may include dual cyclonic separators 6202a, 6202b. Each of the cyclonic separators 6202a, 6202b may include an inlet 6204, a cap 6206, an outlet 6208, an exit 6210, a cover 6212 defining a “C” shaped slot 6214, and a tissue reception container 6216.

The collection assembly 6200 may include a body 6218 which may define a pair of cavities. Each of the cavities may form the tissue reception container 6216. The body 6218 may define a stepped configuration around each of the tissue reception containers 6216 towards its superior side. Each cover 6212 may be received in the stepped configuration over the tissue reception container 6216. The cap 6206 may be disposed over the cover 6212, and engaged to the body 6218. The cap 6206 may define a hollow cylindrical inner body, which may be in fluidic communication with the tissue reception container 6216 via the slot 6214 defined in the cover 6212. The inlet 6204 may be connected to the cap 6206 tangentially such that fluid is received into the cap 6212 tangentially from the inlet 6204. The outlet 6208 may be connected to the cap 6206 towards its superior side. The outlet 6208 of the cyclonic separators 6202a may be connected to the inlet 6204 of the cyclonic separators 6202b via the connecting tube 3120. The tissue reception container 6216 may define a conical portion extending inferiorly. The tissue reception container 6216 may be connected to the exit 6210 at its inferior end. The exits 6210 of the cyclonic separators 6202a, 6202b may be connected.

The fluid which may have tissue may enter the cyclonic separators 6202a through the inlet 6204. The fluid may be received into the cap 6206 tangentially from the inlet 6204, which may result in a cyclone. The tissue which may be heavier than the fluid may advance downwards into the tissue reception container 6216 through the slot 6214, while the fluid or most of the fluid may advance upwards into the outlet 6208. The tissue received by the tissue reception container 6216 may exit the same from the exit 6210. The fluid exiting the cyclonic separators 6202a from the outlet 6208 may include tissues that may have failed to advance downwards into the tissue reception container 6216. Such tissue along with the fluid may enter the inlet 6204 of the cyclonic separators 6202b via the connecting tube 3120. Similar separation may be take place in the cyclonic separators 6202b.

In an embodiment, tissue may be collected by adopting optical means. The tissue extracted from underneath the skin may pass through a channel. An optical means may be configured to analyze the tissues passing through the channel. The optical means may be configured to identify, for example, number of hair shafts in a hair follicle or a follicular unit that has been extracted. The optical means may send signals to one or more actuators based on the identification. The actuators may open one or more gates based on, for example, number of shafts in the extracted unit. Therefore, the optical means may enable separation of extracted units based on, for example, number of shafts present in extracted units.

Referring to FIGS. 63A-63B, a targeting system 6300 may be adapted with the system 100. The targeting system 6300 may be a microscope. The targeting system 6300 may be received in the holder or collar 3526 provided in the arm 2508. The targeting system 6300 may pivot when the arm 2508 is pivoted. As recited earlier the arm 2508 may pivot about the longitudinal axis of the first member 200. The targeting system 6300 may capture images or video while extracting the tissue. The video may capture at least a portion of the counter pressure device 3602. Hence, a user may be able to see hair extending out of the external surface 11 of the skin 10. The hair may be visible within the targeting aperture 3604 of the counter pressure device 3602. Additionally, the piercing members 2502 extending into the skin 10 may be captured in the video. The counter pressure device 3602, the first member 200 and the targeting system 6300 may be aligned such that first member 200 extracts or cores the hair follicle corresponding to the hair that is visible within the targeting aperture 3604. The video may be displayed in a display unit. The targeting system 6300 may be connected to the display unit via a wired connection 6304. The display unit may be positioned at a distance from the system 100, 110. Alternatively, the display unit 6308 may be mounted or engaged to the system 110. Alternatively, the display unit may be mounted on or engaged to the system 100. The display unit 6308 may be mounted over a projection 6306 provided in the system 110. The display unit 6308 may configured such that a user may change the alignment of the display unit 6308 as per the user's convenience.

1. A system comprising:
a targeting system configured to capture at least one image of at least one hair extending out of an external surface of a skin;
a first member configured to extract a hair follicle corresponding to the hair extending out of the external surface of the skin; and
a targeting aperture configured such that the hair extending out of the external surface of the skin is visible within the targeting aperture.

2. The system of claim 1, further comprising a counter pressure device configured to interface with the external surface of the skin such that the hair follicle is disposed between the first member and the counter pressure device, wherein the targeting aperture is defined in the counter pressure device.

3. The system of claim 1, further comprising a holder, wherein the targeting system is engaged with the holder, wherein the holder is adapted to pivot about a longitudinal axis of the first member.

4. The system of claim 1, further comprising a display unit, wherein the display unit is configured to display the image captured by the targeting system.

5. The system of claim 1, the targeting system is a microscope.

6. A system comprising:
a first member defining a first bore, wherein,
the first member comprises a longitudinal axis;
at least a portion of the first member is configured to be moved below an external surface of a skin;
the first member is configured to translate at least in a first direction along the longitudinal axis through a tissue; and
the first member is configured to rotate about the longitudinal axis; and
a counter pressure device configured to interface with at least the external surface of the skin, wherein, the tissue is disposed between the first member and the counter pressure device, wherein, the translation and rotation of the first member result in cutting at least a portion of the tissue.

7. The system of claim 6, further comprising a second member defining a second bore, wherein,
the second bore is configured to receive at least a portion of the first member;
the second member is configured to translate at least in the first direction along the longitudinal axis of the first member;
the second member is configured to clip at least a portion of the tissue which is cut by the first member.

8. The system of claim 7, wherein the second member is configured to clip the tissue that is disposed within the first bore.

9. The system of claim 8, wherein,
the second member comprises at least one arm provided at a distal end of the second member;
the first member defines at least aperture extending from an external surface of the first member into the first bore;
the translation of the second member in the first direction results in the arm translating into the aperture and towards the longitudinal axis of the first member.

10. The system of claim 7, wherein the first member and the second member are configured such that relative translatory motion between the first member and the second member is restricted at least after the second member has traversed a maximum distance in the first direction, until at least the second member and the first member have traversed a preconfigured distance in a second direction opposite to the first direction.

11. The system of claim 7, further comprising a first arm, a second arm, a first connecting member, a second connecting member and at least one paddle, wherein,
the first connecting member connects the first arm with the first member;
the second connecting member connects the second arm with the second member; and
the paddle is engaged with at least one of the first arm and the second arm, such that,
movement of the paddle from an initial position to reach an intermediate position results in translatory motion of the first arm, the first member, the first connecting member, the second arm, the second member and the second connecting member in the first direction towards the tissue;
the translatory motion in the first direction of at least the first member is restricted once the paddle reaches an intermediate position; and
movement of the paddle past the intermediate position to reach a final position results in further translatory motion of the second arm, the second member and the second connecting member in the first direction.

12. The system of claim 11, wherein movement of the paddle from the final position to the initial position results in:
simultaneous translatory motion of the first member and the second member in a second direction, which is opposite to the first direction, until the first member reaches a first member intermediate position;
the second member translates in the second direction while the first member is retained in the first member intermediate position, to assume a second member retracted position; and
the first member and the second member translate in the second direction after the second member has assumed the second member retracted position.

13. The system of claim 12, further comprising a locking assembly wherein, the locking assembly is configured to assume a locking member retracted position and a locking member extended position, wherein,
the locking assembly is configured to allow relative translatory motion between the first member and the second member in the second direction when the locking assembly is in the locking member retracted position; and
the locking assembly is configured to restrict relative translatory motion between the first member and the second member in the second direction when the locking assembly is in the locking member extended position.

14. A system for altering alignment of at least one hair follicle, the system comprising at least one piercing member, wherein the piercing member is configured to,
pivot about a pivot axis; and
be operable to translate along a chosen axis, wherein the chosen axis intersects with the pivot axis, wherein the translation of the piercing member in a first direction enables the piercing member to pierce through an external surface of a skin, and translation of the piercing member in a second direction enables the piercing member to retract out of the skin.

15. The system of claim 14, wherein the system comprises a plurality of piercing members, wherein the plurality of piercing members are spaced such that at least a part of the hair follicle extending below the external surface of the skin is disposed between at least two of the plurality of piercing members when the piercing member are translated into the skin along the chosen axis.

16. The system of claim 14, wherein the piercing member is configured to at least partially rotate about a rotational axis, wherein the rotational axis intersects with the pivot axis.

17. The system of claim 16, further comprising a guide member and an arm, wherein the guide member comprises a plurality of engagement shapes, wherein each of the plurality of engagement shapes is equidistant from the rotational axis, wherein the arm is configured to be operable to at least partially rotate about the rotational axis, wherein the arm is configured to be operable to engage with at least one of a chosen engagement shape, wherein the arm is adapted with the piercing member such that the piercing member rotates with the arm.

18. The system of claim 14, wherein,
the piercing member is configured to translate in the first direction to assume an extended position, wherein the piercing member is configured to be operably locked in the extended position; and
the piercing member is configured to translate in the second direction to assume a retracted position, wherein the piercing member is configured to be operably locked in the retracted position.

19. The system of claim 18, further comprising a knob and a shaft, wherein the piercing member is connected to the shaft and the knob is configured with the shaft, wherein the knob is operable to translate the shaft along a longitudinal axis of the shaft to configure the extent to which the piercing member is extended into the external surface of the skin when the piercing member is locked in the extended position.

20. The system of claim 18, further comprising:
a shaft, wherein the piercing member is connected to the shaft;
an engagement member comprising at least one projection portion, wherein the engagement member is engaged with the shaft; and
a second engagement member defining at least one retracted position lock slot and at least one extended position lock slot, wherein the engagement member is received by the retracted position lock slot to lock the piercing members in the retracted position, and the engagement member is received by the extended position lock slot to lock the piercing members in the extended position.
